SEQUENCE LISTING (579 sequences)

SEQ ID NO: 1 - RNA, 20 nt, Homo sapiens
uagcaccauuugaaaucggu

SEQ ID NO: 2 - RNA, 24 nt, Homo sapiens
uguucauaauacaaaggugcuaau

SEQ ID NO: 3 - RNA, 24 nt, Homo sapiens
uugaagaauguugauggugcuaga

SEQ ID NO: 4 - RNA, 24 nt, Pan trogolodytes
uguucauaauacaaaggugcuaau

SEQ ID NO: 5 - RNA, 24 nt, Pan trogolodytes
uugaagaauguugauggugcuaga

SEQ ID NO: 6 - RNA, 24 nt, Mus musculus
ugcuugcaacacaaaggugcuaau

SEQ ID NO: 7 - RNA, 24 nt, Mus musculus
uugaagaauaugaacggugcugga

SEQ ID NO: 8 - RNA, 22 nt, Rattus norvegicus
cuuucgacacaaaggugcuaau

SEQ ID NO: 9 - RNA, 24 nt, Rattus norvegicus
uugaagaauguggauggagcuaga

SEQ ID NO: 10 - RNA, 23 nt, Canis familiaris
uguucacaauacaaaggugcuaa

SEQ ID NO: 11 - RNA, 24 nt, Canis familiaris
uugaagaacguugauggugcuaga

SEQ ID NO: 12 - RNA, 24 nt, Gorilla gorilla
cacucagaauauaguggugcuaau

SEQ ID NO: 13 - RNA, 21 nt, Gorilla gorilla
uugaauuuugauggugcuagc

SEQ ID NO: 14 - RNA, 24 nt, Fugu rubripes
uguccugucuggaaaggugcucac

SEQ ID NO: 15 - RNA, 19 nt, Fugu rubripes
aagcagagauggugcuaau

SEQ ID NO: 16 - RNA, 18 nt, Danio rerio
uccuguuucaaaggugcu

SEQ ID NO: 17 - RNA, 24 nt, Danio rerio
gcagugauauuauauggugcuaaa

SEQ ID NO: 18 - RNA, 22 nt, Homo sapiens
aaaaugucucaauggugcuaua

SEQ ID NO: 19 - RNA, 24 nt, Homo sapiens
aaagacgcauguuauggugcuaau

SEQ ID NO: 20 - RNA, 22 nt, Pan trogolodytes
aaaaugucucaauggugcuaua

SEQ ID NO: 21 - RNA, 24 nt, Pan trogolodytes
aaagacgcauguuauggugcuaau

SEQ ID NO: 22 - RNA, 22 nt, Mus musculus
aaaaugucucaauggugcuaua

SEQ ID NO: 23 - RNA, 24 nt, Mus musculus
aaagacacauguuaaggugcuaau

SEQ ID NO: 24 - RNA, 22 nt, Rattus norvegicus
auaaugucucaauggugcuaua

SEQ ID NO: 25 - RNA, 24 nt, Rattus norvegicus
aaagauacauguuaaaaugcuaau

SEQ ID NO: 26 - RNA, 22 nt, Canis familiaris
aaaaugucucaauggugcuaua

SEQ ID NO: 27 - RNA, 24 nt, Canis familiaris
aaagacacauguuauggugcuaau

SEQ ID NO: 28 - RNA, 24 nt, Gorilla gorilla
agaacacaucuccguggugcuaua

SEQ ID NO: 29 - RNA, 24 nt, Gorilla gorilla
aaagacuaauaugauggugcuaau

SEQ ID NO: 30 - RNA, 22 nt, Homo sapiens
aaugucacaacauggugcuacu

SEQ ID NO: 31 - RNA, 22 nt, Homo sapiens
cagaaaaccaaagggugcuagg

SEQ ID NO: 32 - RNA, 22 nt, Pan trogolodytes
aaugucacaacauggugcuacu

SEQ ID NO: 33 - RNA, 22 nt, Pan trogolodytes
cagaaaaccaaagggugcuagg

SEQ ID NO: 34 - RNA, 22 nt, Mus musculus
accgucacaacauggugcuacu

SEQ ID NO: 35 - RNA, 21 nt, Mus musculus
aagaaacccaaaggugcuagg

SEQ ID NO: 36 - RNA, 22 nt, Rattus norvegicus
gccgucacaacauggugcuacu

SEQ ID NO: 37 - RNA, 21 nt, Rattus norvegicus
aagaaacccaaaggugcuagg

SEQ ID NO: 38 - RNA, 21 nt, Canis familiaris
aacgaucaacauggugcuacu

SEQ ID NO: 39 - RNA, 22 nt, Canis familiaris
cagaaaaccaaagggugcuagg

SEQ ID NO: 40 - RNA, 24 nt, Gorilla gorilla
uauuucacacaauauggugcuauu

SEQ ID NO: 41 - RNA, 23 nt, Gorilla gorilla
cagaaaaaugcaaaggugcuagg

SEQ ID NO: 42 - RNA, 21 nt, Homo sapiens
ugguaccuauuuggugcuagu

SEQ ID NO: 43 - RNA, 24 nt, Homo sapiens
gugaggguuuguaauggugcuuau

SEQ ID NO: 44 - RNA, 24 nt, Pan trogolodytes
gugaggguuuguaauggugcuuau

SEQ ID NO: 45 - RNA, 21 nt, Mus musculus
uguugucuguuuggugcuagu

SEQ ID NO: 46 - RNA, 24 nt, Mus musculus
gugaggguuuguaauggugcuuau

SEQ ID NO: 47 - RNA, 21 nt, Rattus norvegicus
ugcugucuguuuggugcuagu

SEQ ID NO: 48 - RNA, 24 nt, Rattus norvegicus
gugaggguuuguaauggugcuuau

SEQ ID NO: 49 - RNA, 21 nt, Canis familiaris
uaauaucuauuuggugcuagu

SEQ ID NO: 50 - RNA, 24 nt, Canis familiaris
gugaggguuuguaauggugcuuau

SEQ ID NO: 51 - RNA, 21 nt, Gorilla gorilla
ugauauccauuuggugcuagu

SEQ ID NO: 52 - RNA, 24 nt, Gorilla gorilla
gugaggguuuguaauggugcuuau

SEQ ID NO: 53 - RNA, 24 nt, Fugu rubripes
uaauucauauuuacuggugcuagc

SEQ ID NO: 54 - RNA, 24 nt, Fugu rubripes
agagggguuuguaauggugcuuau

SEQ ID NO: 55 - RNA, 12 nt, Homo sapiens
gaauggugcuuc

SEQ ID NO: 56 - RNA, 24 nt, Homo sapiens
acucucauuuaaacuggugcuuua

SEQ ID NO: 57 - RNA, 24 nt, Homo sapiens
uaaugaguuuuccauggugcuaca

SEQ ID NO: 58 - RNA, 12 nt, Pan trogolodytes
gaauggugcuuc

SEQ ID NO: 59 - RNA, 24 nt, Pan trogolodytes
acucucauuuaaacuggugcuuua

SEQ ID NO: 60 - RNA, 24 nt, Pan trogolodytes
uaaugaguuuuccauggugcuaca

SEQ ID NO: 61 - RNA, 12 nt, Mus musculus
gaagggugccuc

SEQ ID NO: 62 - RNA, 22 nt, Mus musculus
aauccuguuuaaacuggugcug

SEQ ID NO: 63 - RNA, 24 nt, Mus musculus
ucaugcauuuuccauggugcuaca

SEQ ID NO: 64 - RNA, 10 nt, Rattus norvegicus
cgaagaugcc

SEQ ID NO: 65 - RNA, 22 nt, Rattus norvegicus
aguccuguuccaacuggugcua

SEQ ID NO: 66 - RNA, 24 nt, Rattus norvegicus
uaaugcauuuuccacagggcuaca

SEQ ID NO: 67 - RNA, 12 nt, Canis familiaris
gaguggugucuc

SEQ ID NO: 68 - RNA, 24 nt, Canis familiaris
aaucuuauuuaaacuggugcuuua

SEQ ID NO: 69 - RNA, 24 nt, Canis familiaris
ugaugccucuuccauggugcuaca

SEQ ID NO: 70 - RNA, 12 nt, Gorilla gorilla
gagcucuuucuu

SEQ ID NO: 71 - DNA, 31 bp, Artificial Sequence
  Description of Artificial Sequence Synthetic oligonucleotide
ttctcgtgttccgtttgtactctaaggtgga

SEQ ID NO: 72 - DNA, 17 bp, Artificial Sequence
  Description of Artificial Sequence Synthetic oligonucleotide
ctgtaggcaccatcaat

SEQ ID NO: 73 - DNA, 17 bp, Artificial Sequence
  Synthetic oligonucleotide
atcgtaggcaccugaaa

SEQ ID NO: 74 - DNA, 15 bp, Artificial Sequence
  Description of Artificial Sequence Synthetic primer
attgatggtgcctac

SEQ ID NO: 75 - DNA, 81 bp, Artificial Sequence
  Description of Artificial Sequence Synthetic primer
ggccagtgaattgtaatacgactcactatagggttctcgtgttccgtttgtactctaaggtggaatcgtaggcacctgaaa

SEQ ID NO: 76 - DNA, 17 bp, Artificial Sequence
  Description of Artificial Sequence Synthetic primer
attgatggtgcctacag

SEQ ID NO: 77 - DNA, 22 bp, Homo sapiens
tgaggtagtaggttgtatagtt

SEQ ID NO: 78 - DNA, 44 bp, Artificial Sequence
  Description of Artificial Sequence Synthetic probe
aactatacaacctactacctcaaactatacaacctactacctca

SEQ ID NO: 79 - DNA, 22 bp, Homo sapiens
tgaggtagtaggttgtgtggtt

SEQ ID NO: 80 - DNA, 44 bp, Artificial Sequence
  Description of Artificial Sequence Synthetic probe
aaccacacaacctactacctcaaaccacacaacctactacctca

SEQ ID NO: 81 - DNA, 22 bp, Homo sapiens
tgaggtagtaggttgtatggtt

SEQ ID NO: 82 - DNA, 44 bp, Artificial Sequence
  Description of Artificial Sequence Synthetic probe
aaccatacaacctactacctcaaaccatacaacctactacctca

SEQ ID NO: 83 - DNA, 21 bp, Homo sapiens
agaggtagtaggttgcatagt

SEQ ID NO: 84 - DNA, 42 bp, Artificial Sequence
  Description of Artificial Sequence Synthetic probe
actatgcaacctactacctctactatgcaacctactacctct

SEQ ID NO: 85 - DNA, 21 bp, Homo sapiens
tgaggtaggaggttgtatagt

SEQ ID NO: 86 - DNA, 42 bp, Artificial Sequence
  Description of Artificial Sequence Synthetic probe
actatacaacctcctacctcaactatacaacctcctacctca

SEQ ID NO: 87 - DNA, 22 bp, Homo sapiens
tgaggtagtagattgtatagtt

SEQ ID NO: 88 - DNA, 44 bp, Artificial Sequence
  Description of Artificial Sequence Synthetic probe
aactatacaatctactacctcaaactatacaatctactacctca

SEQ ID NO: 89 - DNA, 21 bp, Homo sapiens
tgaggtagtagtttgtacagt

SEQ ID NO: 90 - DNA, 42 bp, Artificial Sequence
  Description of Artificial Sequence Synthetic probe
actgtacaaactactacctcaactgtacaaactactacctca

SEQ ID NO: 91 - DNA, 21 bp, Homo sapiens
tgaggtagtagtttgtgctgt

SEQ ID NO: 92 - DNA, 42 bp, Artificial Sequence
  Description of Artificial Sequence Synthetic probe
acagcacaaactactacctcaacagcacaaactactacctca

SEQ ID NO: 93 - DNA, 21 bp, Homo sapiens
tggaatgtaaagaagtatgta

SEQ ID NO: 94 - DNA, 42 bp, Artificial Sequence
  Description of Artificial Sequence Synthetic probe
tacatacttctttacattccatacatacttctttacattcca

SEQ ID NO: 95 - DNA, 22 bp, Homo sapiens
tggaagactagtgattttgttg

SEQ ID NO: 96 - DNA, 44 bp, Artificial Sequence
  Description of Artificial Sequence Synthetic probe
caacaaaatcactagtcttccacaacaaaatcactagtcttcca

SEQ ID NO: 97 - DNA, 23 bp, Homo sapiens
tctttggttatctagctgtatga

SEQ ID NO: 98 - DNA, 46 bp, Artificial Sequence
  Description of Artificial Sequence Synthetic probe
tcatacagctagataaccaaagatcatacagctagataaccaaaga

SEQ ID NO: 99 - DNA, 21 bp, Homo sapiens
taaagctagataaccgaaagt

SEQ ID NO: 100 - DNA, 42 bp, Artificial Sequence
  Description of Artificial Sequence Synthetic probe
actttcggttatctagctttaactttcggttatctagcttta

SEQ ID NO: 101 - DNA, 23 bp, Homo sapiens
taccctgtagatccgaatttgtg

SEQ ID NO: 102 - DNA, 46 bp, Artificial Sequence
  Description of Artificial Sequence Synthetic probe
cacaaattcggatctacagggtacacaaattcggatctacagggta

SEQ ID NO: 103 - DNA, 22 bp, Homo sapiens
taccctgtagaaccgaatttgt

SEQ ID NO: 104 - DNA, 44 bp, Artificial Sequence
  Description of Artificial Sequence Synthetic probe
acaaattcggttctacagggtaacaaattcggttctacagggta

SEQ ID NO: 105 - DNA, 22 bp, Homo sapiens
tagcagcacataatggtttgtg

SEQ ID NO: 106 - DNA, 44 bp, Artificial Sequence
  Description of Artificial Sequence Synthetic probe
cacaaaccattatgtgctgctacacaaaccattatgtgctgcta

SEQ ID NO: 107 - DNA, 22 bp, Homo sapiens
tagcagcacatcatggtttaca

SEQ ID NO: 108 - DNA, 44 bp, Artificial Sequence
  Description of Artificial Sequence Synthetic probe
tgtaaaccatgatgtgctgctatgtaaaccatgatgtgctgcta

SEQ ID NO: 109 - DNA, 22 bp, Homo sapiens
tagcagcacgtaaatattggcg

SEQ ID NO: 110 - DNA, 44 bp, Artificial Sequence
  Description of Artificial Sequence Synthetic probe
cgccaatatttacgtgctgctacgccaatatttacgtgctgcta

SEQ ID NO: 111 - DNA, 20 bp, Homo sapiens
actgcagtgaaggcacttgt

SEQ ID NO: 112 - DNA, 40 bp, Artificial Sequence
  Description of Artificial Sequence Synthetic probe
acaagtgccttcactgcagtacaagtgccttcactgcagt

SEQ ID NO: 113 - DNA, 24 bp, Homo sapiens
caaagtgcttacagtgcaggtagt

SEQ ID NO: 114 - DNA, 48 bp, Artificial Sequence
  Description of Artificial Sequence Synthetic probe
actacctgcactgtaagcactttgactacctgcactgtaagcactttg

SEQ ID NO: 115 - DNA, 22 bp, Homo sapiens
taaggtgcatctagtgcagata

SEQ ID NO: 116 - DNA, 44 bp, Artificial Sequence
  Description of Artificial Sequence Synthetic probe
tatctgcactagatgcaccttatatctgcactagatgcacctta

SEQ ID NO: 117 - DNA, 23 bp, Homo sapiens
tgtgcaaatctatgcaaaactga

SEQ ID NO: 118 - DNA, 46 bp, Artificial Sequence
  Description of Artificial Sequence Synthetic probe
tcagttttgcatagatttgcacatcagttttgcatagatttgcaca

SEQ ID NO: 119 - DNA, 23 bp, Homo sapiens
tgtgcaaatccatgcaaaactga

SEQ ID NO: 120 - DNA, 46 bp, Artificial Sequence
  Description of Artificial Sequence Synthetic probe
tcagttttgcatggatttgcacatcagttttgcatggatttgcaca

SEQ ID NO: 121 - DNA, 23 bp, Homo sapiens
taaagtgcttatagtgcaggtag

SEQ ID NO: 122 - DNA, 46 bp, Artificial Sequence
  Description of Artificial Sequence Synthetic probe
ctacctgcactataagcactttactacctgcactataagcacttta

SEQ ID NO: 123 - DNA, 22 bp, Homo sapiens
tagcttatcagactgatgttga

SEQ ID NO: 124 - DNA, 44 bp, Artificial Sequence
  Description of Artificial Sequence Synthetic probe
tcaacatcagtctgataagctatcaacatcagtctgataagcta

SEQ ID NO: 125 - DNA, 22 bp, Homo sapiens
aagctgccagttgaagaactgt

SEQ ID NO: 126 - DNA, 44 bp, Artificial Sequence
  Description of Artificial Sequence Synthetic probe
acagttcttcaactggcagcttacagttcttcaactggcagctt

SEQ ID NO: 127 - DNA, 21 bp, Homo sapiens
atcacattgccagggatttcc

SEQ ID NO: 128 - DNA, 42 bp, Artificial Sequence
  Description of Artificial Sequence Synthetic probe
ggaaatccctggcaatgtgatggaaatccctggcaatgtgat

SEQ ID NO: 129 - DNA, 21 bp, Homo sapiens
atcacattgccagggattacc

SEQ ID NO: 130 - DNA, 42 bp, Artificial Sequence
  Description of Artificial Sequence Synthetic probe
ggtaatccctggcaatgtgatggtaatccctggcaatgtgat

SEQ ID NO: 131 - DNA, 22 bp, Homo sapiens
tggctcagttcagcaggaacag

SEQ ID NO: 132 - DNA, 44 bp, Artificial Sequence
  Description of Artificial Sequence Synthetic probe
ctgttcctgctgaactgagccactgttcctgctgaactgagcca

SEQ ID NO: 133 - DNA, 22 bp, Homo sapiens
cattgcacttgtctcggtctga

SEQ ID NO: 134 - DNA, 44 bp, Artificial Sequence
  Description of Artificial Sequence Synthetic probe
tcagaccgagacaagtgcaatgtcagaccgagacaagtgcaatg

SEQ ID NO: 135 - DNA, 21 bp, Homo sapiens
ttcaagtaatccaggataggc

SEQ ID NO: 136 - DNA, 42 bp, Artificial Sequence
  Description of Artificial Sequence Synthetic probe
gcctatcctggattacttgaagcctatcctggattacttgaa

SEQ ID NO: 137 - DNA, 22 bp, Homo sapiens
ttcaagtaattcaggataggtt

SEQ ID NO: 138 - DNA, 44 bp, Artificial Sequence
  Description of Artificial Sequence Synthetic probe
aacctatcctgaattacttgaaaacctatcctgaattacttgaa

SEQ ID NO: 139 - DNA, 21 bp, Homo sapiens
ttcacagtggctaagttccgc

SEQ ID NO: 140 - DNA, 42 bp, Artificial Sequence
  Description of Artificial Sequence Synthetic probe
gcggaacttagccactgtgaagcggaacttagccactgtgaa

SEQ ID NO: 141 - DNA, 21 bp, Homo sapiens
ttcacagtggctaagttctgc

SEQ ID NO: 142 - DNA, 42 bp, Artificial Sequence
  Description of Artificial Sequence Synthetic probe
gcagaacttagccactgtgaagcagaacttagccactgtgaa

SEQ ID NO: 143 - DNA, 22 bp, Homo sapiens
aaggagctcacagtctattgag

SEQ ID NO: 144 - DNA, 44 bp, Artificial Sequence
  Description of Artificial Sequence Synthetic probe
ctcaatagactgtgagctccttctcaatagactgtgagctcctt

SEQ ID NO: 145 - DNA, 21 bp, Homo sapiens
tagcaccatctgaaatcggtt

SEQ ID NO: 146 - DNA, 42 bp, Artificial Sequence
  Description of Artificial Sequence Synthetic probe
aaccgatttcagatggtgctaaaccgatttcagatggtgcta

SEQ ID NO: 147 - DNA, 23 bp, Homo sapiens
tagcaccatttgaaatcagtgtt

SEQ ID NO: 148 - DNA, 46 bp, Artificial Sequence
  Description of Artificial Sequence Synthetic probe
aacactgatttcaaatggtgctaaacactgatttcaaatggtgcta

SEQ ID NO: 149 - DNA, 20 bp, Homo sapiens
tagcaccatttgaaatcggt

SEQ ID NO: 150 - DNA, 40 bp, Artificial Sequence
  Description of Artificial Sequence Synthetic probe
accgatttcaaatggtgctaaccgatttcaaatggtgcta

SEQ ID NO: 151 - DNA, 22 bp, Homo sapiens
ctttcagtcggatgtttgcagc

SEQ ID NO: 152 - DNA, 44 bp, Artificial Sequence
  Description of Artificial Sequence Synthetic probe
gctgcaaacatccgactgaaaggctgcaaacatccgactgaaag

SEQ ID NO: 153 - DNA, 22 bp, Homo sapiens
tgtaaacatcctcgactggaag

SEQ ID NO: 154 - DNA, 44 bp, Artificial Sequence
  Description of Artificial Sequence Synthetic probe
cttccagtcgaggatgtttacacttccagtcgaggatgtttaca

SEQ ID NO: 155 - DNA, 22 bp, Homo sapiens
tgtaaacatcctacactcagct

SEQ ID NO: 156 - DNA, 44 bp, Artificial Sequence
  Description of Artificial Sequence Synthetic probe
agctgagtgtaggatgtttacaagctgagtgtaggatgtttaca

SEQ ID NO: 157 - DNA, 23 bp, Homo sapiens
tgtaaacatcctacactctcagc

SEQ ID NO: 158 - DNA, 46 bp, Artificial Sequence
  Description of Artificial Sequence Synthetic probe
gctgagagtgtaggatgtttacagctgagagtgtaggatgtttaca

SEQ ID NO: 159 - DNA, 22 bp, Homo sapiens
tgtaaacatccccgactggaag

SEQ ID NO: 160 - DNA, 44 bp, Artificial Sequence
  Description of Artificial Sequence Synthetic probe
cttccagtcggggatgtttacacttccagtcggggatgtttaca

SEQ ID NO: 161 - DNA, 22 bp, Homo sapiens
ctttcagtcggatgtttacagc

SEQ ID NO: 162 - DNA, 44 bp, Artificial Sequence
  Description of Artificial Sequence Synthetic probe
gctgtaaacatccgactgaaaggctgtaaacatccgactgaaag

SEQ ID NO: 163 - DNA, 20 bp, Homo sapiens
tgtaaacatccttgactgga

SEQ ID NO: 164 - DNA, 40 bp, Artificial Sequence
  Description of Artificial Sequence Synthetic probe
tccagtcaaggatgtttacatccagtcaaggatgtttaca

SEQ ID NO: 165 - DNA, 21 bp, Homo sapiens
ggcaagatgctggcatagctg

SEQ ID NO: 166 - DNA, 42 bp, Artificial Sequence
  Description of Artificial Sequence Synthetic probe
cagctatgccagcatcttgcccagctatgccagcatcttgcc

SEQ ID NO: 167 - DNA, 21 bp, Homo sapiens
tattgcacattactaagttgc

SEQ ID NO: 168 - DNA, 42 bp, Artificial Sequence
  Description of Artificial Sequence Synthetic probe
gcaacttagtaatgtgcaatagcaacttagtaatgtgcaata

SEQ ID NO: 169 - DNA, 19 bp, Homo sapiens
gtgcattgtagttgcattg

SEQ ID NO: 170 - DNA, 38 bp, Artificial Sequence
  Description of Artificial Sequence Synthetic probe
caatgcaactacaatgcaccaatgcaactacaatgcac

SEQ ID NO: 171 - DNA, 23 bp, Homo sapiens
tggcagtgtcttagctggttgtt

SEQ ID NO: 172 - DNA, 46 bp, Artificial Sequence
  Description of Artificial Sequence Synthetic probe
aacaaccagctaagacactgccaaacaaccagctaagacactgcca

SEQ ID NO: 173 - DNA, 23 bp, Homo sapiens
taggcagtgtcattagctgattg

SEQ ID NO: 174 - DNA, 46 bp, Artificial Sequence
  Description of Artificial Sequence Synthetic probe
caatcagctaatgacactgcctacaatcagctaatgacactgccta

SEQ ID NO: 175 - DNA, 23 bp, Homo sapiens
aggcagtgtagttagctgattgc

SEQ ID NO: 176 - DNA, 46 bp, Artificial Sequence
  Description of Artificial Sequence Synthetic probe
gcaatcagctaactacactgcctgcaatcagctaactacactgcct

SEQ ID NO: 177 - DNA, 21 bp, Homo sapiens
tattgcacttgtcccggcctg

SEQ ID NO: 178 - DNA, 42 bp, Artificial Sequence
  Description of Artificial Sequence Synthetic probe
caggccgggacaagtgcaatacaggccgggacaagtgcaata

SEQ ID NO: 179 - DNA, 22 bp, Homo sapiens
aaagtgctgttcgtgcaggtag

SEQ ID NO: 180 - DNA, 44 bp, Artificial Sequence
  Description of Artificial Sequence Synthetic probe
ctacctgcacgaacagcactttctacctgcacgaacagcacttt

SEQ ID NO: 181 - DNA, 22 bp, Homo sapiens
ttcaacgggtatttattgagca

SEQ ID NO: 182 - DNA, 44 bp, Artificial Sequence
  Description of Artificial Sequence Synthetic probe
tgctcaataaatacccgttgaatgctcaataaatacccgttgaa

SEQ ID NO: 183 - DNA, 22 bp, Homo sapiens
tttggcactagcacatttttgc

SEQ ID NO: 184 - DNA, 44 bp, Artificial Sequence
  Description of Artificial Sequence Synthetic probe
gcaaaaatgtgctagtgccaaagcaaaaatgtgctagtgccaaa

SEQ ID NO: 185 - DNA, 22 bp, Homo sapiens
tgaggtagtaagttgtattgtt

SEQ ID NO: 186 - DNA, 44 bp, Artificial Sequence
  Description of Artificial Sequence Synthetic probe
aacaatacaacttactacctcaaacaatacaacttactacctca

SEQ ID NO: 187 - DNA, 22 bp, Homo sapiens
aacccgtagatccgatcttgtg

SEQ ID NO: 188 - DNA, 44 bp, Artificial Sequence
  Description of Artificial Sequence Synthetic probe
cacaagatcggatctacgggttcacaagatcggatctacgggtt

SEQ ID NO: 189 - DNA, 22 bp, Homo sapiens
cacccgtagaaccgaccttgcg

SEQ ID NO: 190 - DNA, 44 bp, Artificial Sequence
  Description of Artificial Sequence Synthetic probe
cgcaaggtcggttctacgggtgcgcaaggtcggttctacgggtg

SEQ ID NO: 191 - DNA, 22 bp, Homo sapiens
aacccgtagatccgaacttgtg

SEQ ID NO: 192 - DNA, 44 bp, Artificial Sequence
  Description of Artificial Sequence Synthetic probe
cacaagttcggatctacgggttcacaagttcggatctacgggtt

SEQ ID NO: 193 - DNA, 22 bp, Homo sapiens
tacagtactgtgataactgaag

SEQ ID NO: 194 - DNA, 44 bp, Artificial Sequence
  Description of Artificial Sequence Synthetic probe
cttcagttatcacagtactgtacttcagttatcacagtactgta

SEQ ID NO: 195 - DNA, 23 bp, Homo sapiens
agcagcattgtacagggctatga

SEQ ID NO: 196 - DNA, 46 bp, Artificial Sequence
  Description of Artificial Sequence Synthetic probe
tcatagccctgtacaatgctgcttcatagccctgtacaatgctgct

SEQ ID NO: 197 - DNA, 20 bp, Homo sapiens
tcaaatgctcagactcctgt

SEQ ID NO: 198 - DNA, 40 bp, Artificial Sequence
  Description of Artificial Sequence Synthetic probe
acaggagtctgagcatttgaacaggagtctgagcatttga

SEQ ID NO: 199 - DNA, 24 bp, Homo sapiens
aaaagtgcttacagtgcaggtagc

SEQ ID NO: 200 - DNA, 48 bp, Artificial Sequence
  Description of Artificial Sequence Synthetic probe
gctacctgcactgtaagcacttttgctacctgcactgtaagcactttt

SEQ ID NO: 201 - DNA, 21 bp, Homo sapiens
taaagtgctgacagtgcagat

SEQ ID NO: 202 - DNA, 42 bp, Artificial Sequence
  Description of Artificial Sequence Synthetic probe
atctgcactgtcagcactttaatctgcactgtcagcacttta

SEQ ID NO: 203 - DNA, 23 bp, Homo sapiens
agcagcattgtacagggctatca

SEQ ID NO: 204 - DNA, 46 bp, Artificial Sequence
  Description of Artificial Sequence Synthetic probe
tgatagccctgtacaatgctgcttgatagccctgtacaatgctgct

SEQ ID NO: 205 - DNA, 21 bp, Homo sapiens
ataaggatttttaggggcatt

SEQ ID NO: 206 - DNA, 42 bp, Artificial Sequence
  Description of Artificial Sequence Synthetic probe
aatgcccctaaaaatccttataatgcccctaaaaatccttat

SEQ ID NO: 207 - DNA, 23 bp, Homo sapiens
tggagtgtgacaatggtgtttgt

SEQ ID NO: 208 - DNA, 46 bp, Artificial Sequence
  Description of Artificial Sequence Synthetic probe
acaaacaccattgtcacactccaacaaacaccattgtcacactcca

SEQ ID NO: 209 - DNA, 22 bp, Homo sapiens
ttaaggcacgcggtgaatgcca

SEQ ID NO: 210 - DNA, 44 bp, Artificial Sequence
  Description of Artificial Sequence Synthetic probe
tggcattcaccgcgtgccttaatggcattcaccgcgtgccttaa

SEQ ID NO: 211 - DNA, 23 bp, Homo sapiens
tccctgagaccctttaacctgtg

SEQ ID NO: 212 - DNA, 46 bp, Artificial Sequence
  Description of Artificial Sequence Synthetic probe
cacaggttaaagggtctcagggacacaggttaaagggtctcaggga

SEQ ID NO: 213 - DNA, 22 bp, Homo sapiens
tccctgagaccctaacttgtga

SEQ ID NO: 214 - DNA, 44 bp, Artificial Sequence
  Description of Artificial Sequence Synthetic probe
tcacaagttagggtctcagggatcacaagttagggtctcaggga

SEQ ID NO: 215 - DNA, 21 bp, Homo sapiens
tcgtaccgtgagtaataatgc

SEQ ID NO: 216 - DNA, 42 bp, Artificial Sequence
  Description of Artificial Sequence Synthetic probe
gcattattactcacggtacgagcattattactcacggtacga

SEQ ID NO: 217 - DNA, 21 bp, Homo sapiens
cattattacttttggtacgcg

SEQ ID NO: 218 - DNA, 42 bp, Artificial Sequence
  Description of Artificial Sequence Synthetic probe
cgcgtaccaaaagtaataatgcgcgtaccaaaagtaataatg

SEQ ID NO: 219 - DNA, 22 bp, Homo sapiens
tcggatccgtctgagcttggct

SEQ ID NO: 220 - DNA, 44 bp, Artificial Sequence
  Description of Artificial Sequence Synthetic probe
agccaagctcagacggatccgaagccaagctcagacggatccga

SEQ ID NO: 221 - DNA, 22 bp, Homo sapiens
tcacagtgaaccggtctctttt

SEQ ID NO: 222 - DNA, 44 bp, Artificial Sequence
  Description of Artificial Sequence Synthetic probe
aaaagagaccggttcactgtgaaaaagagaccggttcactgtga

SEQ ID NO: 223 - DNA, 22 bp, Homo sapiens
tcacagtgaaccggtctctttc

SEQ ID NO: 224 - DNA, 44 bp, Artificial Sequence
  Description of Artificial Sequence Synthetic probe
gaaagagaccggttcactgtgagaaagagaccggttcactgtga

SEQ ID NO: 225 - DNA, 21 bp, Homo sapiens
ctttttgcggtctgggcttgc

SEQ ID NO: 226 - DNA, 42 bp, Artificial Sequence
  Description of Artificial Sequence Synthetic probe
gcaagcccagaccgcaaaaaggcaagcccagaccgcaaaaag

SEQ ID NO: 227 - DNA, 22 bp, Homo sapiens
cagtgcaatgttaaaagggcat

SEQ ID NO: 228 - DNA, 44 bp, Artificial Sequence
  Description of Artificial Sequence Synthetic probe
atgcccttttaacattgcactgatgcccttttaacattgcactg

SEQ ID NO: 229 - DNA, 22 bp, Homo sapiens
cagtgcaatgatgaaagggcat

SEQ ID NO: 230 - DNA, 44 bp, Artificial Sequence
  Description of Artificial Sequence Synthetic probe
atgccctttcatcattgcactgatgccctttcatcattgcactg

SEQ ID NO: 231 - DNA, 22 bp, Homo sapiens
taacagtctacagccatggtcg

SEQ ID NO: 232 - DNA, 44 bp, Artificial Sequence
  Description of Artificial Sequence Synthetic probe
cgaccatggctgtagactgttacgaccatggctgtagactgtta

SEQ ID NO: 233 - DNA, 22 bp, Homo sapiens
ttggtccccttcaaccagctgt

SEQ ID NO: 234 - DNA, 44 bp, Artificial Sequence
  Description of Artificial Sequence Synthetic probe
acagctggttgaaggggaccaaacagctggttgaaggggaccaa

SEQ ID NO: 235 - DNA, 21 bp, Homo sapiens
ttggtccccttcaaccagcta

SEQ ID NO: 236 - DNA, 42 bp, Artificial Sequence
  Description of Artificial Sequence Synthetic probe
tagctggttgaaggggaccaatagctggttgaaggggaccaa

SEQ ID NO: 237 - DNA, 21 bp, Homo sapiens
tgtgactggttgaccagaggg

SEQ ID NO: 238 - DNA, 42 bp, Artificial Sequence
  Description of Artificial Sequence Synthetic probe
ccctctggtcaaccagtcacaccctctggtcaaccagtcaca

SEQ ID NO: 239 - DNA, 23 bp, Homo sapiens
tatggctttttattcctatgtga

SEQ ID NO: 240 - DNA, 46 bp, Artificial Sequence
  Description of Artificial Sequence Synthetic probe
tcacataggaataaaaagccatatcacataggaataaaaagccata

SEQ ID NO: 241 - DNA, 22 bp, Homo sapiens
tatggcttttcattcctatgtg

SEQ ID NO: 242 - DNA, 44 bp, Artificial Sequence
  Description of Artificial Sequence Synthetic probe
cacataggaatgaaaagccatacacataggaatgaaaagccata

SEQ ID NO: 243 - DNA, 23 bp, Homo sapiens
actccatttgttttgatgatgga

SEQ ID NO: 244 - DNA, 46 bp, Artificial Sequence
  Description of Artificial Sequence Synthetic probe
tccatcatcaaaacaaatggagttccatcatcaaaacaaatggagt

SEQ ID NO: 245 - DNA, 22 bp, Homo sapiens
tattgcttaagaatacgcgtag

SEQ ID NO: 246 - DNA, 44 bp, Artificial Sequence
  Description of Artificial Sequence Synthetic probe
ctacgcgtattcttaagcaatactacgcgtattcttaagcaata

SEQ ID NO: 247 - DNA, 17 bp, Homo sapiens
agctggtgttgtgaatc

SEQ ID NO: 248 - DNA, 34 bp, Artificial Sequence
  Description of Artificial Sequence Synthetic probe
gattcacaacaccagctgattcacaacaccagct

SEQ ID NO: 249 - DNA, 18 bp, Homo sapiens
tctacagtgcacgtgtct

SEQ ID NO: 250 - DNA, 36 bp, Artificial Sequence
  Description of Artificial Sequence Synthetic probe
agacacgtgcactgtagaagacacgtgcactgtaga

SEQ ID NO: 251 - DNA, 21 bp, Homo sapiens
agtggttttaccctatggtag

SEQ ID NO: 252 - DNA, 42 bp, Artificial Sequence
  Description of Artificial Sequence Synthetic probe
ctaccatagggtaaaaccactctaccatagggtaaaaccact

SEQ ID NO: 253 - DNA, 22 bp, Homo sapiens
taacactgtctggtaaagatgg

SEQ ID NO: 254 - DNA, 44 bp, Artificial Sequence
  Description of Artificial Sequence Synthetic probe
ccatctttaccagacagtgttaccatctttaccagacagtgtta

SEQ ID NO: 255 - DNA, 23 bp, Homo sapiens
tgtagtgtttcctactttatgga

SEQ ID NO: 256 - DNA, 46 bp, Artificial Sequence
  Description of Artificial Sequence Synthetic probe
tccataaagtaggaaacactacatccataaagtaggaaacactaca

SEQ ID NO: 257 - DNA, 20 bp, Homo sapiens
cataaagtagaaagcactac

SEQ ID NO: 258 - DNA, 40 bp, Artificial Sequence
  Description of Artificial Sequence Synthetic probe
gtagtgctttctactttatggtagtgctttctactttatg

SEQ ID NO: 259 - DNA, 22 bp, Homo sapiens
tgagatgaagcactgtagctca

SEQ ID NO: 260 - DNA, 44 bp, Artificial Sequence
  Description of Artificial Sequence Synthetic probe
tgagctacagtgcttcatctcatgagctacagtgcttcatctca

SEQ ID NO: 261 - DNA, 22 bp, Homo sapiens
tacagtatagatgatgtactag

SEQ ID NO: 262 - DNA, 44 bp, Artificial Sequence
  Description of Artificial Sequence Synthetic probe
ctagtacatcatctatactgtactagtacatcatctatactgta

SEQ ID NO: 263 - DNA, 24 bp, Homo sapiens
gtccagttttcccaggaatccctt

SEQ ID NO: 264 - DNA, 48 bp, Artificial Sequence
  Description of Artificial Sequence Synthetic probe
aagggattcctgggaaaactggacaagggattcctgggaaaactggac

SEQ ID NO: 265 - DNA, 22 bp, Homo sapiens
tgagaactgaattccatgggtt

SEQ ID NO: 266 - DNA, 44 bp, Artificial Sequence
  Description of Artificial Sequence Synthetic probe
aacccatggaattcagttctcaaacccatggaattcagttctca

SEQ ID NO: 267 - DNA, 20 bp, Homo sapiens
gtgtgtggaaatgcttctgc

SEQ ID NO: 268 - DNA, 40 bp, Artificial Sequence
  Description of Artificial Sequence Synthetic probe
gcagaagcatttccacacacgcagaagcatttccacacac

SEQ ID NO: 269 - DNA, 22 bp, Homo sapiens
tcagtgcactacagaactttgt

SEQ ID NO: 270 - DNA, 44 bp, Artificial Sequence
  Description of Artificial Sequence Synthetic probe
acaaagttctgtagtgcactgaacaaagttctgtagtgcactga

SEQ ID NO: 271 - DNA, 22 bp, Homo sapiens
tcagtgcatcacagaactttgt

SEQ ID NO: 272 - DNA, 44 bp, Artificial Sequence
  Description of Artificial Sequence Synthetic probe
acaaagttctgtgatgcactgaacaaagttctgtgatgcactga

SEQ ID NO: 273 - DNA, 22 bp, Homo sapiens
tctggctccgtgtcttcactcc

SEQ ID NO: 274 - DNA, 44 bp, Artificial Sequence
  Description of Artificial Sequence Synthetic probe
ggagtgaagacacggagccagaggagtgaagacacggagccaga

SEQ ID NO: 275 - DNA, 22 bp, Homo sapiens
tctcccaacccttgtaccagtg

SEQ ID NO: 276 - DNA, 44 bp, Artificial Sequence
  Description of Artificial Sequence Synthetic probe
cactggtacaagggttgggagacactggtacaagggttgggaga

SEQ ID NO: 277 - DNA, 22 bp, Homo sapiens
actagactgaagctccttgagg

SEQ ID NO: 278 - DNA, 44 bp, Artificial Sequence
  Description of Artificial Sequence Synthetic probe
cctcaaggagcttcagtctagtcctcaaggagcttcagtctagt

SEQ ID NO: 279 - DNA, 22 bp, Homo sapiens
tcagtgcatgacagaacttggg

SEQ ID NO: 280 - DNA, 44 bp, Artificial Sequence
  Description of Artificial Sequence Synthetic probe
cccaagttctgtcatgcactgacccaagttctgtcatgcactga

SEQ ID NO: 281 - DNA, 20 bp, Homo sapiens
ttgcatagtcacaaaagtga

SEQ ID NO: 282 - DNA, 40 bp, Artificial Sequence
  Description of Artificial Sequence Synthetic probe
tcacttttgtgactatgcaatcacttttgtgactatgcaa

SEQ ID NO: 283 - DNA, 22 bp, Homo sapiens
taggttatccgtgttgccttcg

SEQ ID NO: 284 - DNA, 44 bp, Artificial Sequence
  Description of Artificial Sequence Synthetic probe
cgaaggcaacacggataacctacgaaggcaacacggataaccta

SEQ ID NO: 285 - DNA, 22 bp, Homo sapiens
aatcatacacggttgacctatt

SEQ ID NO: 286 - DNA, 44 bp, Artificial Sequence
  Description of Artificial Sequence Synthetic probe
aataggtcaaccgtgtatgattaataggtcaaccgtgtatgatt

SEQ ID NO: 287 - DNA, 22 bp, Homo sapiens
ttaatgctaatcgtgatagggg

SEQ ID NO: 288 - DNA, 44 bp, Artificial Sequence
  Description of Artificial Sequence Synthetic probe
cccctatcacgattagcattaacccctatcacgattagcattaa

SEQ ID NO: 289 - DNA, 23 bp, Homo sapiens
aacattcaacgctgtcggtgagt

SEQ ID NO: 290 - DNA, 46 bp, Artificial Sequence
  Description of Artificial Sequence Synthetic probe
actcaccgacagcgttgaatgttactcaccgacagcgttgaatgtt

SEQ ID NO: 291 - DNA, 22 bp, Homo sapiens
aacattcattgctgtcggtggg

SEQ ID NO: 292 - DNA, 44 bp, Artificial Sequence
  Description of Artificial Sequence Synthetic probe
cccaccgacagcaatgaatgttcccaccgacagcaatgaatgtt

SEQ ID NO: 293 - DNA, 22 bp, Homo sapiens
aacattcaacctgtcggtgagt

SEQ ID NO: 294 - DNA, 44 bp, Artificial Sequence
  Description of Artificial Sequence Synthetic probe
actcaccgacaggttgaatgttactcaccgacaggttgaatgtt

SEQ ID NO: 295 - DNA, 22 bp, Homo sapiens
tttggcaatggtagaactcaca

SEQ ID NO: 296 - DNA, 44 bp, Artificial Sequence
  Description of Artificial Sequence Synthetic probe
tgtgagttctaccattgccaaatgtgagttctaccattgccaaa

SEQ ID NO: 297 - DNA, 21 bp, Homo sapiens
tggttctagacttgccaacta

SEQ ID NO: 298 - DNA, 42 bp, Artificial Sequence
  Description of Artificial Sequence Synthetic probe
tagttggcaagtctagaaccatagttggcaagtctagaacca

SEQ ID NO: 299 - DNA, 23 bp, Homo sapiens
tatggcactggtagaattcactg

SEQ ID NO: 300 - DNA, 46 bp, Artificial Sequence
  Description of Artificial Sequence Synthetic probe
cagtgaattctaccagtgccatacagtgaattctaccagtgccata

SEQ ID NO: 301 - DNA, 22 bp, Homo sapiens
tggacggagaactgataagggt

SEQ ID NO: 302 - DNA, 44 bp, Artificial Sequence
  Description of Artificial Sequence Synthetic probe
acccttatcagttctccgtccaacccttatcagttctccgtcca

SEQ ID NO: 303 - DNA, 18 bp, Homo sapiens
tggagagaaaggcagttc

SEQ ID NO: 304 - DNA, 36 bp, Artificial Sequence
  Description of Artificial Sequence Synthetic probe
gaactgcctttctctccagaactgcctttctctcca

SEQ ID NO: 305 - DNA, 23 bp, Homo sapiens
caaagaattctccttttgggctt

SEQ ID NO: 306 - DNA, 46 bp, Artificial Sequence
  Description of Artificial Sequence Synthetic probe
aagcccaaaaggagaattctttgaagcccaaaaggagaattctttg

SEQ ID NO: 307 - DNA, 21 bp, Homo sapiens
tcgtgtcttgtgttgcagccg

SEQ ID NO: 308 - DNA, 42 bp, Artificial Sequence
  Description of Artificial Sequence Synthetic probe
cggctgcaacacaagacacgacggctgcaacacaagacacga

SEQ ID NO: 309 - DNA, 22 bp, Homo sapiens
catcccttgcatggtggagggt

SEQ ID NO: 310 - DNA, 44 bp, Artificial Sequence
  Description of Artificial Sequence Synthetic probe
accctccaccatgcaagggatgaccctccaccatgcaagggatg

SEQ ID NO: 311 - DNA, 23 bp, Homo sapiens
gtgcctactgagctgatatcagt

SEQ ID NO: 312 - DNA, 46 bp, Artificial Sequence
  Description of Artificial Sequence Synthetic probe
actgatatcagctcagtaggcacactgatatcagctcagtaggcac

SEQ ID NO: 313 - DNA, 22 bp, Homo sapiens
tgatatgtttgatatattaggt

SEQ ID NO: 314 - DNA, 44 bp, Artificial Sequence
  Description of Artificial Sequence Synthetic probe
acctaatatatcaaacatatcaacctaatatatcaaacatatca

SEQ ID NO: 315 - DNA, 22 bp, Homo sapiens
caacggaatcccaaaagcagct

SEQ ID NO: 316 - DNA, 44 bp, Artificial Sequence
  Description of Artificial Sequence Synthetic probe
agctgcttttgggattccgttgagctgcttttgggattccgttg

SEQ ID NO: 317 - DNA, 21 bp, Homo sapiens
ctgacctatgaattgacagcc

SEQ ID NO: 318 - DNA, 42 bp, Artificial Sequence
  Description of Artificial Sequence Synthetic probe
ggctgtcaattcataggtcagggctgtcaattcataggtcag

SEQ ID NO: 319 - DNA, 21 bp, Homo sapiens
aactggcctacaaagtcccag

SEQ ID NO: 320 - DNA, 42 bp, Artificial Sequence
  Description of Artificial Sequence Synthetic probe
ctgggactttgtaggccagttctgggactttgtaggccagtt

SEQ ID NO: 321 - DNA, 22 bp, Homo sapiens
tgtaacagcaactccatgtgga

SEQ ID NO: 322 - DNA, 44 bp, Artificial Sequence
  Description of Artificial Sequence Synthetic probe
tccacatggagttgctgttacatccacatggagttgctgttaca

SEQ ID NO: 323 - DNA, 21 bp, Homo sapiens
tagcagcacagaaatattggc

SEQ ID NO: 324 - DNA, 42 bp, Artificial Sequence
  Description of Artificial Sequence Synthetic probe
gccaatatttctgtgctgctagccaatatttctgtgctgcta

SEQ ID NO: 325 - DNA, 21 bp, Homo sapiens
taggtagtttcatgttgttgg

SEQ ID NO: 326 - DNA, 42 bp, Artificial Sequence
  Description of Artificial Sequence Synthetic probe
ccaacaacatgaaactacctaccaacaacatgaaactaccta

SEQ ID NO: 327 - DNA, 21 bp, Homo sapiens
taggtagtttcctgttgttgg

SEQ ID NO: 328 - DNA, 42 bp, Artificial Sequence
  Description of Artificial Sequence Synthetic probe
ccaacaacaggaaactacctaccaacaacaggaaactaccta

SEQ ID NO: 329 - DNA, 22 bp, Homo sapiens
ttcaccaccttctccacccagc

SEQ ID NO: 330 - DNA, 44 bp, Artificial Sequence
  Description of Artificial Sequence Synthetic probe
gctgggtggagaaggtggtgaagctgggtggagaaggtggtgaa

SEQ ID NO: 331 - DNA, 19 bp, Homo sapiens
ggtccagaggggagatagg

SEQ ID NO: 332 - DNA, 38 bp, Artificial Sequence
  Description of Artificial Sequence Synthetic probe
cctatctcccctctggacccctatctcccctctggacc

SEQ ID NO: 333 - DNA, 23 bp, Homo sapiens
cccagtgttcagactacctgttc

SEQ ID NO: 334 - DNA, 46 bp, Artificial Sequence
  Description of Artificial Sequence Synthetic probe
gaacaggtagtctgaacactggggaacaggtagtctgaacactggg

SEQ ID NO: 335 - DNA, 22 bp, Homo sapiens
tacagtagtctgcacattggtt

SEQ ID NO: 336 - DNA, 44 bp, Artificial Sequence
  Description of Artificial Sequence Synthetic probe
aaccaatgtgcagactactgtaaaccaatgtgcagactactgta

SEQ ID NO: 337 - DNA, 23 bp, Homo sapiens
cccagtgtttagactatctgttc

SEQ ID NO: 338 - DNA, 46 bp, Artificial Sequence
  Description of Artificial Sequence Synthetic probe
gaacagatagtctaaacactggggaacagatagtctaaacactggg

SEQ ID NO: 339 - DNA, 22 bp, Homo sapiens
taacactgtctggtaacgatgt

SEQ ID NO: 340 - DNA, 44 bp, Artificial Sequence
  Description of Artificial Sequence Synthetic probe
acatcgttaccagacagtgttaacatcgttaccagacagtgtta

SEQ ID NO: 341 - DNA, 23 bp, Homo sapiens
taatactgcctggtaatgatgac

SEQ ID NO: 342 - DNA, 46 bp, Artificial Sequence
  Description of Artificial Sequence Synthetic probe
gtcatcattaccaggcagtattagtcatcattaccaggcagtatta

SEQ ID NO: 343 - DNA, 22 bp, Homo sapiens
taatactgccgggtaatgatgg

SEQ ID NO: 344 - DNA, 44 bp, Artificial Sequence
  Description of Artificial Sequence Synthetic probe
ccatcattacccggcagtattaccatcattacccggcagtatta

SEQ ID NO: 345 - DNA, 22 bp, Homo sapiens
gtgaaatgtttaggaccactag

SEQ ID NO: 346 - DNA, 44 bp, Artificial Sequence
  Description of Artificial Sequence Synthetic probe
ctagtggtcctaaacatttcacctagtggtcctaaacatttcac

SEQ ID NO: 347 - DNA, 22 bp, Homo sapiens
ttccctttgtcatcctatgcct

SEQ ID NO: 348 - DNA, 44 bp, Artificial Sequence
  Description of Artificial Sequence Synthetic probe
aggcataggatgacaaagggaaaggcataggatgacaaagggaa

SEQ ID NO: 349 - DNA, 22 bp, Homo sapiens
tccttcattccaccggagtctg

SEQ ID NO: 350 - DNA, 44 bp, Artificial Sequence
  Description of Artificial Sequence Synthetic probe
cagactccggtggaatgaaggacagactccggtggaatgaagga

SEQ ID NO: 351 - DNA, 22 bp, Homo sapiens
tggaatgtaaggaagtgtgtgg

SEQ ID NO: 352 - DNA, 44 bp, Artificial Sequence
  Description of Artificial Sequence Synthetic probe
ccacacacttccttacattccaccacacacttccttacattcca

SEQ ID NO: 353 - DNA, 22 bp, Homo sapiens
ataagacgagcaaaaagcttgt

SEQ ID NO: 354 - DNA, 44 bp, Artificial Sequence
  Description of Artificial Sequence Synthetic probe
acaagctttttgctcgtcttatacaagctttttgctcgtcttat

SEQ ID NO: 355 - DNA, 22 bp, Homo sapiens
ctgtgcgtgtgacagcggctga

SEQ ID NO: 356 - DNA, 44 bp, Artificial Sequence
  Description of Artificial Sequence Synthetic probe
tcagccgctgtcacacgcacagtcagccgctgtcacacgcacag

SEQ ID NO: 357 - DNA, 22 bp, Homo sapiens
ttccctttgtcatccttcgcct

SEQ ID NO: 358 - DNA, 44 bp, Artificial Sequence
  Description of Artificial Sequence Synthetic probe
aggcgaaggatgacaaagggaaaggcgaaggatgacaaagggaa

SEQ ID NO: 359 - DNA, 21 bp, Homo sapiens
taacagtctccagtcacggcc

SEQ ID NO: 360 - DNA, 42 bp, Artificial Sequence
  Description of Artificial Sequence Synthetic probe
ggccgtgactggagactgttaggccgtgactggagactgtta

SEQ ID NO: 361 - DNA, 22 bp, Homo sapiens
accatcgaccgttgattgtacc

SEQ ID NO: 362 - DNA, 44 bp, Artificial Sequence
  Description of Artificial Sequence Synthetic probe
ggtacaatcaacggtcgatggtggtacaatcaacggtcgatggt

SEQ ID NO: 363 - DNA, 21 bp, Homo sapiens
acagcaggcacagacaggcag

SEQ ID NO: 364 - DNA, 42 bp, Artificial Sequence
  Description of Artificial Sequence Synthetic probe
ctgcctgtctgtgcctgctgtctgcctgtctgtgcctgctgt

SEQ ID NO: 365 - DNA, 21 bp, Homo sapiens
atgacctatgaattgacagac

SEQ ID NO: 366 - DNA, 42 bp, Artificial Sequence
  Description of Artificial Sequence Synthetic probe
gtctgtcaattcataggtcatgtctgtcaattcataggtcat

SEQ ID NO: 367 - DNA, 21 bp, Homo sapiens
taatctcagctggcaactgtg

SEQ ID NO: 368 - DNA, 42 bp, Artificial Sequence
  Description of Artificial Sequence Synthetic probe
cacagttgccagctgagattacacagttgccagctgagatta

SEQ ID NO: 369 - DNA, 24 bp, Homo sapiens
tactgcatcaggaactgattggat

SEQ ID NO: 370 - DNA, 48 bp, Artificial Sequence
  Description of Artificial Sequence Synthetic probe
atccaatcagttcctgatgcagtaatccaatcagttcctgatgcagta

SEQ ID NO: 371 - DNA, 21 bp, Homo sapiens
ttgtgcttgatctaaccatgt

SEQ ID NO: 372 - DNA, 42 bp, Artificial Sequence
  Description of Artificial Sequence Synthetic probe
acatggttagatcaagcacaaacatggttagatcaagcacaa

SEQ ID NO: 373 - DNA, 21 bp, Homo sapiens
tgattgtccaaacgcaattct

SEQ ID NO: 374 - DNA, 42 bp, Artificial Sequence
  Description of Artificial Sequence Synthetic probe
agaattgcgtttggacaatcaagaattgcgtttggacaatca

SEQ ID NO: 375 - DNA, 21 bp, Homo sapiens
ccacaccgtatctgacacttt

SEQ ID NO: 376 - DNA, 42 bp, Artificial Sequence
  Description of Artificial Sequence Synthetic probe
aaagtgtcagatacggtgtggaaagtgtcagatacggtgtgg

SEQ ID NO: 377 - DNA, 23 bp, Homo sapiens
agctacattgtctgctgggtttc

SEQ ID NO: 378 - DNA, 46 bp, Artificial Sequence
  Description of Artificial Sequence Synthetic probe
gaaacccagcagacaatgtagctgaaacccagcagacaatgtagct

SEQ ID NO: 379 - DNA, 24 bp, Homo sapiens
agctacatctggctactgggtctc

SEQ ID NO: 380 - DNA, 48 bp, Artificial Sequence
  Description of Artificial Sequence Synthetic probe
gagacccagtagccagatgtagctgagacccagtagccagatgtagct

SEQ ID NO: 381 - DNA, 21 bp, Homo sapiens
tgtcagtttgtcaaatacccc

SEQ ID NO: 382 - DNA, 42 bp, Artificial Sequence
  Description of Artificial Sequence Synthetic probe
ggggtatttgacaaactgacaggggtatttgacaaactgaca

SEQ ID NO: 383 - DNA, 23 bp, Homo sapiens
caagtcactagtggttccgttta

SEQ ID NO: 384 - DNA, 46 bp, Artificial Sequence
  Description of Artificial Sequence Synthetic probe
taaacggaaccactagtgacttgtaaacggaaccactagtgacttg

SEQ ID NO: 385 - DNA, 21 bp, Homo sapiens
agggccccccctcaatcctgt

SEQ ID NO: 386 - DNA, 42 bp, Artificial Sequence
  Description of Artificial Sequence Synthetic probe
acaggattgagggggggccctacaggattgagggggggccct

SEQ ID NO: 387 - DNA, 22 bp, Homo sapiens
tggtttaccgtcccacatacat

SEQ ID NO: 388 - DNA, 44 bp, Artificial Sequence
  Description of Artificial Sequence Synthetic probe
atgtatgtgggacggtaaaccaatgtatgtgggacggtaaacca

SEQ ID NO: 389 - DNA, 23 bp, Homo sapiens
cagtgcaatagtattgtcaaagc

SEQ ID NO: 390 - DNA, 46 bp, Artificial Sequence
  Description of Artificial Sequence Synthetic probe
gctttgacaatactattgcactggctttgacaatactattgcactg

SEQ ID NO: 391 - DNA, 23 bp, Homo sapiens
taagtgcttccatgttttggtga

SEQ ID NO: 392 - DNA, 46 bp, Artificial Sequence
  Description of Artificial Sequence Synthetic probe
tcaccaaaacatggaagcacttatcaccaaaacatggaagcactta

SEQ ID NO: 393 - DNA, 22 bp, Homo sapiens
taaacgtggatgtacttgcttt

SEQ ID NO: 394 - DNA, 44 bp, Artificial Sequence
  Description of Artificial Sequence Synthetic probe
aaagcaagtacatccacgtttaaaagcaagtacatccacgttta

SEQ ID NO: 395 - DNA, 23 bp, Homo sapiens
taagtgcttccatgttttagtag

SEQ ID NO: 396 - DNA, 46 bp, Artificial Sequence
  Description of Artificial Sequence Synthetic probe
ctactaaaacatggaagcacttactactaaaacatggaagcactta

SEQ ID NO: 397 - DNA, 23 bp, Homo sapiens
actttaacatggaagtgctttct

SEQ ID NO: 398 - DNA, 46 bp, Artificial Sequence
  Description of Artificial Sequence Synthetic probe
agaaagcacttccatgttaaagtagaaagcacttccatgttaaagt

SEQ ID NO: 399 - DNA, 23 bp, Homo sapiens
taagtgcttccatgtttcagtgg

SEQ ID NO: 400 - DNA, 46 bp, Artificial Sequence
  Description of Artificial Sequence Synthetic probe
ccactgaaacatggaagcacttaccactgaaacatggaagcactta

SEQ ID NO: 401 - DNA, 22 bp, Homo sapiens
tttaacatgggggtacctgctg

SEQ ID NO: 402 - DNA, 44 bp, Artificial Sequence
  Description of Artificial Sequence Synthetic probe
cagcaggtacccccatgttaaacagcaggtacccccatgttaaa

SEQ ID NO: 403 - DNA, 23 bp, Homo sapiens
taagtgcttccatgtttgagtgt

SEQ ID NO: 404 - DNA, 46 bp, Artificial Sequence
  Description of Artificial Sequence Synthetic probe
acactcaaacatggaagcacttaacactcaaacatggaagcactta

SEQ ID NO: 405 - DNA, 23 bp, Homo sapiens
aaaagctgggttgagagggcgaa

SEQ ID NO: 406 - DNA, 46 bp, Artificial Sequence
  Description of Artificial Sequence Synthetic probe
ttcgccctctcaacccagcttttttcgccctctcaacccagctttt

SEQ ID NO: 407 - DNA, 22 bp, Homo sapiens
gcacattacacggtcgacctct

SEQ ID NO: 408 - DNA, 44 bp, Artificial Sequence
  Description of Artificial Sequence Synthetic probe
agaggtcgaccgtgtaatgtgcagaggtcgaccgtgtaatgtgc

SEQ ID NO: 409 - DNA, 22 bp, Homo sapiens
ccactgccccaggtgctgctgg

SEQ ID NO: 410 - DNA, 44 bp, Artificial Sequence
  Description of Artificial Sequence Synthetic probe
ccagcagcacctggggcagtggccagcagcacctggggcagtgg

SEQ ID NO: 411 - DNA, 23 bp, Homo sapiens
cgcatcccctagggcattggtgt

SEQ ID NO: 412 - DNA, 46 bp, Artificial Sequence
  Description of Artificial Sequence Synthetic probe
acaccaatgccctaggggatgcgacaccaatgccctaggggatgcg

SEQ ID NO: 413 - DNA, 23 bp, Homo sapiens
cctagtaggtgtccagtaagtgt

SEQ ID NO: 414 - DNA, 46 bp, Artificial Sequence
  Description of Artificial Sequence Synthetic probe
acacttactggacacctactaggacacttactggacacctactagg

SEQ ID NO: 415 - DNA, 20 bp, Homo sapiens
cctctgggcccttcctccag

SEQ ID NO: 416 - DNA, 40 bp, Artificial Sequence
  Description of Artificial Sequence Synthetic probe
ctggaggaagggcccagaggctggaggaagggcccagagg

SEQ ID NO: 417 - DNA, 22 bp, Homo sapiens
ctggccctctctgcccttccgt

SEQ ID NO: 418 - DNA, 44 bp, Artificial Sequence
  Description of Artificial Sequence Synthetic probe
acggaagggcagagagggccagacggaagggcagagagggccag

SEQ ID NO: 419 - DNA, 23 bp, Homo sapiens
gcaaagcacacggcctgcagaga

SEQ ID NO: 420 - DNA, 46 bp, Artificial Sequence
  Description of Artificial Sequence Synthetic probe
tctctgcaggccgtgtgctttgctctctgcaggccgtgtgctttgc

SEQ ID NO: 421 - DNA, 21 bp, Homo sapiens
gcccctgggcctatcctagaa

SEQ ID NO: 422 - DNA, 42 bp, Artificial Sequence
  Description of Artificial Sequence Synthetic probe
ttctaggataggcccaggggcttctaggataggcccaggggc

SEQ ID NO: 423 - DNA, 23 bp, Homo sapiens
tcaagagcaataacgaaaaatgt

SEQ ID NO: 424 - DNA, 46 bp, Artificial Sequence
  Description of Artificial Sequence Synthetic probe
acatttttcgttattgctcttgaacatttttcgttattgctcttga

SEQ ID NO: 425 - DNA, 23 bp, Homo sapiens
tccagctcctatatgatgccttt

SEQ ID NO: 426 - DNA, 46 bp, Artificial Sequence
  Description of Artificial Sequence Synthetic probe
aaaggcatcatataggagctggaaaaggcatcatataggagctgga

SEQ ID NO: 427 - DNA, 23 bp, Homo sapiens
tccagcatcagtgattttgttga

SEQ ID NO: 428 - DNA, 46 bp, Artificial Sequence
  Description of Artificial Sequence Synthetic probe
tcaacaaaatcactgatgctggatcaacaaaatcactgatgctgga

SEQ ID NO: 429 - DNA, 21 bp, Homo sapiens
tccctgtcctccaggagctca

SEQ ID NO: 430 - DNA, 42 bp, Artificial Sequence
  Description of Artificial Sequence Synthetic probe
tgagctcctggaggacagggatgagctcctggaggacaggga

SEQ ID NO: 431 - DNA, 23 bp, Homo sapiens
tccgtctcagttactttatagcc

SEQ ID NO: 432 - DNA, 46 bp, Artificial Sequence
  Description of Artificial Sequence Synthetic probe
ggctataaagtaactgagacggaggctataaagtaactgagacgga

SEQ ID NO: 433 - DNA, 24 bp, Homo sapiens
tctcacacagaaatcgcacccgtc

SEQ ID NO: 434 - DNA, 48 bp, Artificial Sequence
  Description of Artificial Sequence Synthetic probe
gacgggtgcgatttctgtgtgagagacgggtgcgatttctgtgtgaga

SEQ ID NO: 435 - DNA, 21 bp, Homo sapiens
tgctgactcctagtccagggc

SEQ ID NO: 436 - DNA, 42 bp, Artificial Sequence
  Description of Artificial Sequence Synthetic probe
gccctggactaggagtcagcagccctggactaggagtcagca

SEQ ID NO: 437 - DNA, 23 bp, Homo sapiens
tgtctgcccgcatgcctgcctct

SEQ ID NO: 438 - DNA, 46 bp, Artificial Sequence
  Description of Artificial Sequence Synthetic probe
agaggcaggcatgcgggcagacaagaggcaggcatgcgggcagaca

SEQ ID NO: 439 - DNA, 22 bp, Homo sapiens
ttatcagaatctccaggggtac

SEQ ID NO: 440 - DNA, 44 bp, Artificial Sequence
  Description of Artificial Sequence Synthetic probe
gtacccctggagattctgataagtacccctggagattctgataa

SEQ ID NO: 441 - DNA, 22 bp, Homo sapiens
aattgcactttagcaatggtga

SEQ ID NO: 442 - DNA, 44 bp, Artificial Sequence
  Description of Artificial Sequence Synthetic probe
tcaccattgctaaagtgcaatttcaccattgctaaagtgcaatt

SEQ ID NO: 443 - DNA, 22 bp, Homo sapiens
acatagaggaaattccacgttt

SEQ ID NO: 444 - DNA, 44 bp, Artificial Sequence
  Description of Artificial Sequence Synthetic probe
aaacgtggaatttcctctatgtaaacgtggaatttcctctatgt

SEQ ID NO: 445 - DNA, 21 bp, Homo sapiens
aataatacatggttgatcttt

SEQ ID NO: 446 - DNA, 42 bp, Artificial Sequence
  Description of Artificial Sequence Synthetic probe
aaagatcaaccatgtattattaaagatcaaccatgtattatt

SEQ ID NO: 447 - DNA, 21 bp, Homo sapiens
gcctgctggggtggaacctgg

SEQ ID NO: 448 - DNA, 42 bp, Artificial Sequence
  Description of Artificial Sequence Synthetic probe
ccaggttccaccccagcaggcccaggttccaccccagcaggc

SEQ ID NO: 449 - DNA, 21 bp, Homo sapiens
gtgccgccatcttttgagtgt

SEQ ID NO: 450 - DNA, 42 bp, Artificial Sequence
  Description of Artificial Sequence Synthetic probe
acactcaaaagatggcggcacacactcaaaagatggcggcac

SEQ ID NO: 451 - DNA, 23 bp, Homo sapiens
aaagtgctgcgacatttgagcgt

SEQ ID NO: 452 - DNA, 46 bp, Artificial Sequence
  Description of Artificial Sequence Synthetic probe
acgctcaaatgtcgcagcactttacgctcaaatgtcgcagcacttt

SEQ ID NO: 453 - DNA, 23 bp, Homo sapiens
gaagtgcttcgattttggggtgt

SEQ ID NO: 454 - DNA, 46 bp, Artificial Sequence
  Description of Artificial Sequence Synthetic probe
acaccccaaaatcgaagcacttcacaccccaaaatcgaagcacttc

SEQ ID NO: 455 - DNA, 22 bp, Homo sapiens
actcaaaatgggggcgctttcc

SEQ ID NO: 456 - DNA, 44 bp, Artificial Sequence
  Description of Artificial Sequence Synthetic probe
ggaaagcgcccccattttgagtggaaagcgcccccattttgagt

SEQ ID NO: 457 - DNA, 22 bp, Homo sapiens
ttataatacaacctgataagtg

SEQ ID NO: 458 - DNA, 44 bp, Artificial Sequence
  Description of Artificial Sequence Synthetic probe
cacttatcaggttgtattataacacttatcaggttgtattataa

SEQ ID NO: 459 - DNA, 22 bp, Homo sapiens
tttgttcgttcggctcgcgtga

SEQ ID NO: 460 - DNA, 44 bp, Artificial Sequence
  Description of Artificial Sequence Synthetic probe
tcacgcgagccgaacgaacaaatcacgcgagccgaacgaacaaa

SEQ ID NO: 461 - DNA, 21 bp, Homo sapiens
atcatagaggaaaatccacgt

SEQ ID NO: 462 - DNA, 42 bp, Artificial Sequence
  Description of Artificial Sequence Synthetic probe
acgtggattttcctctatgatacgtggattttcctctatgat

SEQ ID NO: 463 - DNA, 22 bp, Homo sapiens
atcacacaaaggcaacttttgt

SEQ ID NO: 464 - DNA, 44 bp, Artificial Sequence
  Description of Artificial Sequence Synthetic probe
acaaaagttgcctttgtgtgatacaaaagttgcctttgtgtgat

SEQ ID NO: 465 - DNA, 22 bp, Homo sapiens
ctcctgactccaggtcctgtgt

SEQ ID NO: 466 - DNA, 44 bp, Artificial Sequence
  Description of Artificial Sequence Synthetic probe
acacaggacctggagtcaggagacacaggacctggagtcaggag

SEQ ID NO: 467 - DNA, 19 bp, Homo sapiens
tggtagactatggaacgta

SEQ ID NO: 468 - DNA, 38 bp, Artificial Sequence
  Description of Artificial Sequence Synthetic probe
tacgttccatagtctaccatacgttccatagtctacca

SEQ ID NO: 469 - DNA, 22 bp, Homo sapiens
tatgtaatatggtccacatctt

SEQ ID NO: 470 - DNA, 44 bp, Artificial Sequence
  Description of Artificial Sequence Synthetic probe
aagatgtggaccatattacataaagatgtggaccatattacata

SEQ ID NO: 471 - DNA, 22 bp, Homo sapiens
tggttgaccatagaacatgcgc

SEQ ID NO: 472 - DNA, 44 bp, Artificial Sequence
  Description of Artificial Sequence Synthetic probe
gcgcatgttctatggtcaaccagcgcatgttctatggtcaacca

SEQ ID NO: 473 - DNA, 22 bp, Homo sapiens
tatacaagggcaagctctctgt

SEQ ID NO: 474 - DNA, 44 bp, Artificial Sequence
  Description of Artificial Sequence Synthetic probe
acagagagcttgcccttgtataacagagagcttgcccttgtata

SEQ ID NO: 475 - DNA, 22 bp, Homo sapiens
gaagttgttcgtggtggattcg

SEQ ID NO: 476 - DNA, 44 bp, Artificial Sequence
  Description of Artificial Sequence Synthetic probe
cgaatccaccacgaacaacttccgaatccaccacgaacaacttc

SEQ ID NO: 477 - DNA, 22 bp, Homo sapiens
agatcagaaggtgattgtggct

SEQ ID NO: 478 - DNA, 44 bp, Artificial Sequence
  Description of Artificial Sequence Synthetic probe
agccacaatcaccttctgatctagccacaatcaccttctgatct

SEQ ID NO: 479 - DNA, 20 bp, Homo sapiens
attcctagaaattgttcata

SEQ ID NO: 480 - DNA, 40 bp, Artificial Sequence
  Description of Artificial Sequence Synthetic probe
tatgaacaatttctaggaattatgaacaatttctaggaat

SEQ ID NO: 481 - DNA, 22 bp, Homo sapiens
ctggacttagggtcagaaggcc

SEQ ID NO: 482 - DNA, 44 bp, Artificial Sequence
  Description of Artificial Sequence Synthetic probe
ggccttctgaccctaagtccagggccttctgaccctaagtccag

SEQ ID NO: 483 - DNA, 22 bp, Homo sapiens
ctggacttggagtcagaaggcc

SEQ ID NO: 484 - DNA, 44 bp, Artificial Sequence
  Description of Artificial Sequence Synthetic probe
ggccttctgactccaagtccagggccttctgactccaagtccag

SEQ ID NO: 485 - DNA, 22 bp, Homo sapiens
agctcggtctgaggcccctcag

SEQ ID NO: 486 - DNA, 44 bp, Artificial Sequence
  Description of Artificial Sequence Synthetic probe
ctgaggggcctcagaccgagctctgaggggcctcagaccgagct

SEQ ID NO: 487 - DNA, 22 bp, Homo sapiens
cagcagcaattcatgttttgaa

SEQ ID NO: 488 - DNA, 44 bp, Artificial Sequence
  Description of Artificial Sequence Synthetic probe
ttcaaaacatgaattgctgctgttcaaaacatgaattgctgctg

SEQ ID NO: 489 - DNA, 21 bp, Homo sapiens
atcgggaatgtcgtgtccgcc

SEQ ID NO: 490 - DNA, 42 bp, Artificial Sequence
  Description of Artificial Sequence Synthetic probe
ggcggacacgacattcccgatggcggacacgacattcccgat

SEQ ID NO: 491 - DNA, 22 bp, Drosophila melanogaster
tggaatgtaaagaagtatggag

SEQ ID NO: 492 - DNA, 44 bp, Artificial Sequence
  Description of Artificial Sequence Synthetic probe
ctccatacttctttacattccactccatacttctttacattcca

SEQ ID NO: 493 - DNA, 23 bp, Drosophila melanogaster
tatcacagccagctttgatgagc

SEQ ID NO: 494 - DNA, 46 bp, Artificial Sequence
  Description of Artificial Sequence Synthetic probe
gctcatcaaagctggctgtgatagctcatcaaagctggctgtgata

SEQ ID NO: 495 - DNA, 22 bp, Drosophila melanogaster
tcactgggcaaagtgtgtctca

SEQ ID NO: 496 - DNA, 44 bp, Artificial Sequence
  Description of Artificial Sequence Synthetic probe
tgagacacactttgcccagtgatgagacacactttgcccagtga

SEQ ID NO: 497 - DNA, 21 bp, Drosophila melanogaster
ataaagctagacaaccattga

SEQ ID NO: 498 - DNA, 42 bp, Artificial Sequence
  Description of Artificial Sequence Synthetic probe
tcaatggttgtctagctttattcaatggttgtctagctttat

SEQ ID NO: 499 - DNA, 23 bp, Drosophila melanogaster
aaaggaacgatcgttgtgatatg

SEQ ID NO: 500 - DNA, 46 bp, Artificial Sequence
  Description of Artificial Sequence Synthetic probe
catatcacaacgatcgttcctttcatatcacaacgatcgttccttt

SEQ ID NO: 501 - DNA, 22 bp, Drosophila melanogaster
tatcacagtggctgttcttttt

SEQ ID NO: 502 - DNA, 44 bp, Artificial Sequence
  Description of Artificial Sequence Synthetic probe
aaaaagaacagccactgtgataaaaaagaacagccactgtgata

SEQ ID NO: 503 - DNA, 23 bp, Drosophila melanogaster
tgagatcattttgaaagctgatt

SEQ ID NO: 504 - DNA, 46 bp, Artificial Sequence
  Description of Artificial Sequence Synthetic probe
aatcagctttcaaaatgatctcaaatcagctttcaaaatgatctca

SEQ ID NO: 505 - DNA, 20 bp, Artificial Sequence
  Description of Artificial Sequence Synthetic primer
cccaaggacaagaggcatgt

SEQ ID NO: 506 - DNA, 20 bp, Artificial Sequence
  Description of Artificial Sequence Synthetic primer
ccgccatactcgaactggaa

SEQ ID NO: 507 - DNA, 25 bp, Artificial Sequence
  Description of Artificial Sequence Synthetic primer
gattgagacccttcttactcctgaa

SEQ ID NO: 508 - DNA, 21 bp, Artificial Sequence
  Description of Artificial Sequence Synthetic primer
gggtggctgagtctcaagtca

SEQ ID NO: 509 - DNA, 25 bp, Artificial Sequence
  Description of Artificial Sequence Synthetic primer
tggacagattctagtgctgagaaga

SEQ ID NO: 510 - DNA, 23 bp, Artificial Sequence
  Description of Artificial Sequence Synthetic primer
ttgccgtagctaaactgaaaacc

SEQ ID NO: 511 - DNA, 25 bp, Artificial Sequence
  Description of Artificial Sequence Synthetic primer
gtattttcacacgtaagcacattcg

SEQ ID NO: 512 - DNA, 21 bp, Artificial Sequence
  Description of Artificial Sequence Synthetic primer
ccctgctgaggtctgtgaaca

SEQ ID NO: 513 - DNA, 20 bp, Artificial Sequence
  Description of Artificial Sequence Synthetic primer
gtggccaatcactggtgtca

SEQ ID NO: 514 - DNA, 20 bp, Artificial Sequence
  Description of Artificial Sequence Synthetic primer
cctccattgcattcgatgaa

SEQ ID NO: 515 - DNA, 17 bp, Artificial Sequence
  Description of Artificial Sequence Synthetic primer
ccccgatggctcgaaaa

SEQ ID NO: 516 - DNA, 18 bp, Artificial Sequence
  Description of Artificial Sequence Synthetic primer
tgcggaatggcaaagctt

SEQ ID NO: 517 - DNA, 21 bp, Artificial Sequence
  Description of Artificial Sequence Synthetic primer
ctcgtacctcagcatgccatt

SEQ ID NO: 518 - DNA, 21 bp, Artificial Sequence
  Description of Artificial Sequence Synthetic primer
gccttcactgtccaggatcag

SEQ ID NO: 519 - DNA, 17 bp, Artificial Sequence
  Description of Artificial Sequence Synthetic primer
gccccctgcagctatgg

SEQ ID NO: 520 - DNA, 20 bp, Artificial Sequence
  Description of Artificial Sequence Synthetic primer
ggcctatgcggaagtaacca

SEQ ID NO: 521 - DNA, 22 bp, Artificial Sequence
  Description of Artificial Sequence Synthetic primer
ggaaaagtcttcggtccagtgt

SEQ ID NO: 522 - DNA, 22 bp, Artificial Sequence
  Description of Artificial Sequence Synthetic primer
tatgcaggccagacattcattc

SEQ ID NO: 523 - DNA, 18 bp, Artificial Sequence
  Description of Artificial Sequence Synthetic primer
ccccccaacacgtctctg

SEQ ID NO: 524 - DNA, 19 bp, Artificial Sequence
  Description of Artificial Sequence Synthetic primer
tcacgccgcaagtcttcag

SEQ ID NO: 525 - DNA, 21 bp, Artificial Sequence
  Description of Artificial Sequence Synthetic primer
ccaacgcaaagcaatacatga

SEQ ID NO: 526 - DNA, 22 bp, Artificial Sequence
  Description of Artificial Sequence Synthetic primer
ttttcgcttccctgttttagct

SEQ ID NO: 527 - DNA, 20 bp, Artificial Sequence
  Description of Artificial Sequence Synthetic primer
ttgacgccacagtgggacta

SEQ ID NO: 528 - DNA, 20 bp, Artificial Sequence
  Description of Artificial Sequence Synthetic primer
cagctccaacaattgccaaa

SEQ ID NO: 529 - DNA, 21 bp, Artificial Sequence
  Description of Artificial Sequence Synthetic primer
ctgacgctgacctggttgtct

SEQ ID NO: 530 - DNA, 19 bp, Artificial Sequence
  Description of Artificial Sequence Synthetic primer
ccccggcgatttaactgat

SEQ ID NO: 531 - DNA, 24 bp, Artificial Sequence
  Description of Artificial Sequence Synthetic primer
cacattaggctgttggttcaaact

SEQ ID NO: 532 - DNA, 19 bp, Artificial Sequence
  Description of Artificial Sequence Synthetic primer
caggatgcgctgaccactt

SEQ ID NO: 533 - DNA, 23 bp, Artificial Sequence
  Description of Artificial Sequence Synthetic primer
tcatctgtgattccctcctgcta

SEQ ID NO: 534 - DNA, 21 bp, Artificial Sequence
  Description of Artificial Sequence Synthetic primer
gctggcctttctttcatttcc

SEQ ID NO: 535 - DNA, 22 bp, Artificial Sequence
  Description of Artificial Sequence Synthetic primer
tgcacactcagacctctttgct

SEQ ID NO: 536 - DNA, 22 bp, Artificial Sequence
  Description of Artificial Sequence Synthetic primer
tgtcaggtaagggccagttttt

SEQ ID NO: 537 - DNA, 22 bp, Artificial Sequence
  Description of Artificial Sequence Synthetic primer
tcaacgaccactttgtcaagct

SEQ ID NO: 538 - DNA, 19 bp, Artificial Sequence
  Description of Artificial Sequence Synthetic primer
ccatgaggtccaccaccct

SEQ ID NO: 539 - DNA, 5273 bp, Artificial Sequence
  Description of Artificial Sequence Synthetic polynucleotide
cccgggaggtaccgagctcttacgcgtgctagctcgagatctgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggcattccggtactgttggtaaaatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcactagtctgcagggatccgtcgactaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgaaccatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcctcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 540 - RNA, 1404 nt, Artificial Sequence
  Description of Artificial Sequence Synthetic polynucleotide
acucccuccaucccaaccuggcucccucccacccaaccaacuuuccccccaacccggaaacagacaagcaacccaaacugaacccccucaaaagccaaaaaaugggagacaauuucacauggacuuuggaaaauauuuuuuuccuuugcauucaucucucaaacuuaguuuuuaucuuugaccaaccgaacaugaccaaaaaccaaaagugcauucaaccuuaccaaaaaaaaaaaaaaaaaaagaauaaauaaauaacuuuuuaaaaaaggaagcuugguccacuugcuugaagacccaugcggggguaagucccuuucugcccguugggcuuaugaaaccccaaugcugcccuuucugcuccuuucuccacaccccccuuggggccuccccuccacuccuucccaaaucugucuccccagaagacacaggaaacaauguauugucugcccagcaaucaaaggcaaugcucaaacacccaaguggcccccacccucagcccgcuccugcccgcccagcacccccaggcccugggggaccugggguucucagacugccaaagaagccuugccaucuggcgcucccauggcucuugcaacaucuccccuucguuuuugagggggucaugccgggggagccaccagccccucacuggguucggaggagagucaggaagggccacgacaaagcagaaacaucggauuuggggaacgcgugucaaucccuugugccgcagggcugggcgggagagacuguucuguuccuuguguaacuguguugcugaaagacuaccucguucuugucuugaugugucaccggggcaacugccugggggcggggaugggggcaggguggaagcggcuccccauuuuauaccaaaggugcuacaucuaugugaugggugggguggggagggaaucacuggugcuauagaaauugagaugcccccccaggccagcaaauguuccuuuuuguucaaagucuauuuuuauuccuugauauuuuucuuuuuuuuuuuuuuuuuuuguggauggggacuugugaauuuuucuaaaggugcuauuuaacaugggaggagagcgugugcggcuccagcccagcccgcugcucacuuuccacccucucuccaccugccucuggcuucucaggccucugcucuccgaccucucuccucugaaacccuccuccacagcugcagcccauccucccggcucccuccuagucuguccugcguccucuguccccggguuucagagacaacuucccaaagcacaaagcaguuuuucccccuaggggugggaggaagcaaaagacucuguaccuauuuuguauguguauaauaauuugagauguuuuuaauuauuuugauugcuggaauaaagcauguggaaaugacccaaacau

SEQ ID NO: 541 - RNA, 839 nt, Artificial Sequence
  Description of Artificial Sequence Synthetic polynucleotide
augaacucaaucuaaauuaaaaaagaaagaaauuugaaaaaacuuucucuuugccauuucuucuucuucuuuuuuaacugaaagcugaauccuuccauuucuucugcacaucuacuugcuuaaauugugggcaaaagagaaaaagaaggauugaucagagcauugugcaauacaguuucauuaacuccuucccccgcucccccaaaaauuugaauuuuuuuuucaacacucuuacaccuguuauggaaaaugucaaccuuuguaagaaaaccaaaauaaaaauugaaaaauaaaaaccauaaacauuugcaccacuuguggcuuuugaauaucuuccacagagggaaguuuaaaacccaaacuuccaaagguuuaaacuaccucaaaacacuuucccaugagugugauccacauuguuaggugcugaccuagacagagaugaacugagguccuuguuuuguuuuguucauaauacaaaggugcuaauuaauaguauuucagauacuugaagaauguugauggugcuagaagaauuugagaagaaauacuccuguauugaguuguaucgugugguguauuuuuuaaaaaauuugauuuagcauucauauuuuccaucuuauucccaauuaaaaguaugcagauuauuugcccaaaucuucuucagauucagcauuuguucuuugccagucucauuuucaucuucuuccaugguuccacagaagcuuuguuucuugggcaagcagaaaaauuaaauuguaccuauuuuguauaugugagauguuuaaauaaauugugaaaaaaaugaaauaaagcauguuugguuuuccaaaagaacauau

SEQ ID NO: 542 - RNA, 972 nt, Artificial Sequence
  Description of Artificial Sequence Synthetic polynucleotide
accaaacucuaucugaaaucccaacaaaaaaaauuuaacuccauauguguuccucuuguucuaaucuugucaaccagugcaagugaccgacaaaauuccaguuauuuauuuccaaaauguuuggaaacaguauaauuugacaaagaaaaaugauacuucucuuuuuuugcuguuccaccaaauacaauucaaaugcuuuuuguuuuauuuuuuuaccaauuccaauuucaaaaugucucaauggugcuauaauaaauaaacuucaacacucuuuaugauaacaacacuguguuauauucuuugaauccuagcccaucugcagagcaaugacugugcucaccaguaaaagauaaccuuucuuucugaaauagucaaauacgaaauuagaaaagcccucccuauuuuaacuaccucaacuggucagaaacacagauuguauucuaugagucccagaagaugaaaaaaauuuuauacguugauaaaacuuauaaauuucauugauuaaucuccuggaagauugguuuaaaaagaaaaguguaaugcaagaauuuaaagaaauauuuuuaaagccacaauuauuuuaauauuggauaucaacugcuuguaaaggugcuccucuuuuuucuugucauugcuggucaagauuacuaauauuugggaaggcuuuaaagacgcauguuauggugcuaauguacuuucacuuuuaaacucuagaucagaauuguugacuugcauucagaacauaaaugcacaaaaucuguacaugucucccaucagaaagauucauuggcaugccacaggggauucuccuccuucauccuguaaaggucaacaauaaaaaccaaauuauggggcugcuuuugucacacuagcauagagaauguguugaaauuuaacuuuguaagcuuguaugugguuguugaucuuuuuuuuccuuacagacacccauaauaaaauaucauauuaaaauuc

SEQ ID NO: 543 - RNA, 1399 nt, Artificial Sequence
  Description of Artificial Sequence Synthetic polynucleotide
ugaagccugacucagcuaaugucacaacauggugcuacuucuucuucuuuuuguuaacagcaacgaacccuagaaauauauccuguguaccucacuguccaauaugaaaaccguaaagugccuuauaggaauuugcguaacuaacacacccugcuucauugaccucuacuugcugaaggagaaaaagacagcgauaagcuuucaauaguggcauaccaaauggcacuuuugaugaaauaaaauaucaauauuuucugcaauccaaugcacugaugugugaagugagaacuccaucagaaaaccaaagggugcuaggaggugugggugccuuccauacuguuugcccauuuucauucuuguauuauaauuaauuuucuacccccagagauaaauguuuguuuauaucacugucuagcuguuucaaaauuuaggucccuuggucuguacaaauaauagcaauguaaaaaugguuuuuugaaccuccaaauggaauuacagacucaguagccauaucuuccaaccccccaguauaaauuucugucuuucugcuaugugugguacuuugcagcugcuuuugcagaaaucacaauuuuccuguggaauaaagaugguccaaaaauagucaaaaauuaaauauauauauauauuaguaauuuauauagaugucagcaauuaggcagaucaagguuuaguuuaacuuccacuguuaaaauaaagcuuacauaguuuucuuccuuugaaagacugugcuguccuuuaacauagguuuuuaaagacuaggauauugaaugugaaacauccguuuucauuguucacuucuaaaccaaaaauuauguguugccaaaaccaaacccagguucaugaauauggugucuauuauagugaaacauguacuuugagcuuauuguuuuuauucuguauuaaauauuuucaggguuuuaaacacuaaucacaaacugaaugacuugacuucaaaagcaacaaccuuaaaggccgucauuucauuaguauuccucauucugcauccuggcuugaaaaacagcucuguugaaucacaguaucaguauuuucacacguaagcacauucgggccauuuccgugguuucucaugagcuguguucacagaccucagcagggcaucgcauggaccgcaggagggcagauucggaccacuaggccugaaaugacauuucacuaaaagucuccaaaacauuucuaagacuacuaaggccuuuuauguaauuucuuuaaauguguauuucuuaagaauucaaauuuguaauaaaacuauuuguauaaaaauuaagcuuuuauuaauuuguugcuaguauugccacagacgcauuaaaagaaacuuacugcacaagcugcuaauaaauuuguaagcuuugcauaccuu

SEQ ID NO: 544 - RNA, 833 nt, Artificial Sequence
  Description of Artificial Sequence Synthetic polynucleotide
gccggcgcgugccaggaagggccauuuuggugcuuauucuuaacuuauuaccucaggugccaacccaaaaauugguuuuauuuuuuucuuaaaaaaaaaaaagucuaccaaaggaauuugcauccagcagcagcacuuagaccugccagccacugucaccgagcgggugcaagcacucggggucccuggagggcaagcccugcccacagaaagccaggagcagcccuggcccccaucagcccugcuagacgcaccgccugaaggcacagcuaaccacuucgcacacacccauguaaccacugcacuuuccaaugccacagacaacucacauuguucaacucccuucucggggugggacagacgagacaacagcacacaggcagccagccguggccagaggcucgaggggcucagggccucaggcacccguccccacacgagggccccgugggugggccuggcccugcuuucuacgccaauguuaugccagcuccauguucucccaaauaccguugaugugaauuauuuuaaaggcaaaaccgugcucuuuauuuuaaaaaacacugauaaucacacugcgguaggucauucuuuugccacaucccuauagaccacuggguuuggcaaaacucaggcagaaguggagaccuuucuagacaucauugucagccuugcuacuugaagguacaccccauagggucggaggugcuguccccacugccccacguugucccugagauuuaaccccuccacugcugggggugagcuguacucuucugacugcccccuccuguguaacgacuacaaaauaaaacuugguucugaauauuuuu

SEQ ID NO: 545 - RNA, 900 nt, Artificial Sequence
  Description of Artificial Sequence Synthetic polynucleotide
uggccuucugaugauucuuaaagaguuuucaauuuuuucuuaugugaagaguugacacugaaaucuaaaauguuuaauuguuguaaauauuacaguuuuuuuuuuuuacuacauauucuuuacaacagcaaccaaagaaaacauaccucaauacacucaaaacugaagacauagaggacucagaucaaagacaaaaucugauccauauauuggugcuagauucugcaggaaaccccagcagugugaacgcaucccaacauagguuaagagcaaguugaaaacaaaggccauggcauucugccacugcauccuucagacaguuauauccuccuuuuaaaccauuguuguugaguguaagauguccuucauguuuucuuauaaagucaguguuuagaaauguuacccuuucuaaguuauauacagaucaaaugcuuuuuucuuucacguacauccaucauuugcaacugcuguucguacacagaaacaggacugcucaaaugauccuauuuguauuuucugaugcuaucagacucuaauguuuuuuucccuaaaauauuauugccaucaugcuuuaggaauuuuauauuuuuacacaaucauauuuuaguauggugucuguuuauguaacucugacuugcuggaaaaguugaaacuccaaauaaucugaaacuagaaaagaaauagcacauaauuacuaccuuccccuuggcggcucuccuccccaacccccaccccacaauuuuaugacuuccauuuggcaauuguugaauuauaacugcgacugaaacaaacagguucauagagaugaauuuucugagaaacauauaucuacauguuguauaauuggauuuuuuuuccauguaagugaacauaaaaacaucuuuuccgggugcuuucuuc

SEQ ID NO: 546 - RNA, 1214 nt, Artificial Sequence
  Description of Artificial Sequence Synthetic polynucleotide
aagcggaaaaagaaccaaagaagggcaguucaagcgaccaaaggguggguggaaggugcugcaguaugaauacuguacgaauauuuugacucuggucugaaaagauaaaagaauguuaucgaaaacuacauggaauaauugaagucccuucaaguuugaaaguaagcauuuuaggacaaauaaaaggaaauucaacuuuguacuuguggaaacuaaucccuaaauaugaauagguuuauauugauucauggguaacagguccauaauaaauuauuggaaacuaggaugucugaauaucaaggaagacagccauagucucuuacagugccucuguuggucugucucaaacugaauugggugggaaaagguaugguccaauauaaaaguuccauuuuugccauuauuggcaaaucuugccuuuguuuauuuuggugccaguguuuucugcuuaaucauuugcuuuguuggcaucuguguuuauuuacuuguacaccacaugcaguuuacaucugucuuaacuacuccuucccagguaaauuccaauuauauuugacauccagcuaagagggcccaucucuucucaccucuuuccuagucaguauauucagcaaauauuuauugagcccuuacugugggcaaaucauuguacuggauaauugagaaaaauagauaauucccuuauucaguaaaugucuacugagcacaaucuagugaaucauuacaguauggccucauuguuuuguuugagguguguuauucauaacaauauuuuacaccauucguaucaauguaauuauagaacacaauauacgaucaaggauaaguaauugugugguuaucugccauuuaaaaguauccaguauuugaucacauuauuauaaauaaugaaaaaaugauuuaaucuguaauaaacugguuuauugugcagugacuguaauauacuagaguuauaauaaauuguuuacucugccucaccaaacacaugcuaggauauaacccccaaaauaaguauuuaacuuugcauuagguauaaaggagacugggugcuauaauuagauuauuuugaggcagacagagagcuguuauccuaacugauuuaguauguucuguaauugagaaaauguucaccaaauuauacuuuuuagugauuuacauguacauuuuauaggggacauguucuguguauagcgaauaaauaacuuuuauaguaucac

SEQ ID NO: 547 - RNA, 2059 nt, Artificial Sequence
  Description of Artificial Sequence Synthetic polynucleotide
uaguauaaaccauggucauuuuuaggcauguaucauucauuuacucauaguuugguuuacuuaaauuaucaggaauacaauguugcaaugaugcuuaaaaaacacuuguuaguuuucccuguaccaggcaaugguuauaauuaaaaugauaugcuguugagaagccacucuuaagaguccaguuuguuuaauguuaugggcagcuaccaauuuguggugucucuguauauuuuuguaaagauucucauuuuuuaugcuugaaguauuuggugaaaagauguugguugaccauaauuugcaacauugucucauuaaaaauaaacuuucauauucauauuugguagaacuguuaaccuagaaauguagcuugcuaauaagauagaaugauacaaaagugaaguaguagccacaguacaacacugacugcucagacacauuuagguucaggguggaccuuuaugucuugucaagaugucuaggcccggcugggcgugguggcucacaccuguaaucccagcacuuugggaggccgaggcgggcggaucacgaggucaggaguucgagaccagccugaccaacacggugaaaccccgucucuacuaaaaauacaaaaauuauccgggcaugguggcacaugccuguaaucucagcuacucaggaggcugaggcaagagaaucgcuugaaccugggagguagaaguugcagugagccaaaaucacgccacugcacuccagccugggcaacagagugagacuccgucucaaaaaaaaaaaaaaaccggaugucuaggccaaugauaauuauuuuugaugcaguguggauuaguucuuuuguuaaccccacugucuuggggaaugaugccagcugggaaauugaguuuuugacugaaacauggagccuucacugcuuuuuuucugguuccuaugaagauuuggaacauagaaaacacaaaaacucaccuuaaaauuugagcaggucguugauggcaaaaauaauuuuaaggaaaaaggaauauucuuauguaguuauucuaaaguuuaaggagcguuguugaccauaauauugcuuaguuuucuuacugcuguuaaguaaguaaauuguuucaaaguagguuuugugugugugugccuaguguaaaagaacugaaauuuugaugcuuacagcacuuggcucgugcauuuguaucaaaauuugccugccucuuuaugagggaggccugcuuuucacaccucaguuuauuuaauacgaggcaaguuguaagacaacacucauucuaggugauucuguggugccaugaaauuuaagguaauuuggggaaaaggauuagucaguuuuaagcaagagucacaucuuuugagcuuucgauuaucaguguaguaccugacuaaaaaugaaguaauacccuuaaaccauuuauaauuucuaguauuucucugaaagaucguuuuggggacaaaagugacuugacauguccaauuucauuucagaauaaaaagcuagcaucuuuaaaaaucucagauugcuugcuuacagauacaaguacgaauuauggacaaacgauuccuuuuagaggauuacuuuuuucaauuucgguuuuaguaaucuaggcuuugccuguaaagaauacaacgauggauuuuaaauacuguuuguggaauguguuuaaaggauugauucuagaaccuuuguauauuugauaguauuucuaacuuucauuucuuuacuguuugcaguuaauguucauguucugcuaugcaaucguuuauaugcacguuucuuuaauuuuuuuagauuuuccuggauguauaguuuaaacaacaaaaagucuauuuaaaacuguagcaguaguuuacaguucuagcaaagaggaaaguugugggguuaaacuuuguauuuucuuucuuauagaggcuucuaaaaagguauuuuuauauguucuuuuuaacaaauauuguguacaaccuuuaaaacaucaauguuuggaucaaaacaagacccagcuuauuuucugcuugcuguaaauuaagcaaacaugcuauaauaaaaacaaaaugaagg

SEQ ID NO: 548 - RNA, 200 nt, Artificial Sequence
  Description of Artificial Sequence Synthetic polynucleotide
gaccccuggaccaccagccccagcaagagcacaagaggaagagagagacccucacugcuggggagucccugccacacucagucccccaccacacugaaucuccccuccucacaguugccauguagaccccuugaagaggggaggggccuagggagccgcaccuugucauguaccaucaauaaaguacccugugcucaacc

SEQ ID NO: 549 - RNA, 2793 nt, Artificial Sequence
  Description of Artificial Sequence Synthetic polynucleotide
ugucuuuagggcuggaaggcagcaucccucugacaggggggcaguugugaggccacagagugccuugacacaaagauuacauuuuucagacccccacuccucugcugcuguccaucacuguccuuuugaaccaggaaaagucacagaguuuaaagagaagcaaauuaaacauccugaaucgggaacaaaggguuuuaucuaauaaagugucucuuccaucacguugcuaccuuacccacacuucccucugauuugcgugaggacguggcauccuacuuacguacguggcauaacacaucgugugagcccauguaugcugggguagagcaaguagcccuccccugucucaucgauccagcagaaccuccucagucucaguacucuuguuucuauaaggaaaaguuuugcuacuaacaguagcauugugauggccaguauauccaguccauggauaaagaaaaugcaucugcaucuccugccccucuuccuucuaagcaaaaggaaauaaacauccugugccaaagguauuggucauuuagaaugucgguagccauccaucagugcuuuuagcuauuaugaguguaggacacugagccauccgugggucaggaugcaauuauuuauaaaaguccccaggugaacauggcugaagauuuuucuaguauauuaauaauugacuaggaagaugaacuuuuuuucagaucuuugggcagcugauaauuuaaaucuggaugggcagcuugcacucaccaauagaccaaaagacaucuuuugauauucuuauaaauggaacuuacacagaagaaauagggauaugauaaccacuaaaguuuuguuuucaaaaucaaacuaauucuuacagcuuuuuuauuaguuagucuuggaacuaguguuaaguaucuggcagagaacaguuaaucccuaaggucuugacaaaacagaagaaaaacaagccuccucguccuagucuuuucuagcaaagggauaaaacuuagauggcagcuuguacugucagaaucccguguauccauuuguucuucuguuggagagaugagacauuugacccuuagcuccaguuuucuucugauguuuccaucuuccagaaucccucaaaaaacauuguuugccaaauccugguggcaaauacuugcacucaguauuucacacagcugccaacgcuaucgaguuccugcacuuugugauuuaaauccacucuaaaccuucccucuaaguguagagggaagacccuuacguggaguuuccuagugggcuucucaacuuuugauccucagcucugugguuuuaagaccacagugugacaguucccugccacacacccccuuccuccuaccaacccaccuuugagauucauauauagccuuuaacacuaugcaacuuuguacuuugcguagcaggggcugggguggggggaaagaaaccuauuaucauggacacacuggugcuauuaauuauuucaaauuuauauuuuugugugaauguuuuguguuuuguuuauccaugcuauagaacaaggaauuuauguagauauacuuaguccuauuucuagaaugacacucuguucacuuugcucaauuuuuccucuucacuggcacaaguaucugaauaccuccuucccucccuucuagaguucuuuggauuguacuccaaagaauugugccuuguguuugcagcaucuccauucucuaaauuaauauaauugcuuuccuccacacccagccacguaaagagguaacuuggguccucuuccauugcaguccugaugauccuaaccugcagcacggugguuuuacaauguuccagagcaggaacgccagguugacaagcuaugguaggauuaggaaaguuugcugaagaggaucuuugacgccacagugggacuagccaggaaugagggagaaaugcccuuuuuggcaauuguuggagcuggauagguaaguuuuauaagggaguacauuuugacugagcacuuagggcaucaggaacagugcuacuuacugguggguagacugggagaggugguguaacuuaguucuugaugaucccacuuccuguuuccaucugcuugggauauaccagaguuuaccacaaguguuuugacgauauacuccugagcuuucacucugcuggcuucucccaggccucuucuacuauggcaggagauguggugugcuguugcaaaguuuucacgucaucguuuccuggcuaguucauuucauuaaguggcuacauccuaacauaugcauuggucaagguugcagcaagaggacugaagauugacugccaagcuaguuugggugaaguucacuccagcaagucucaggccacaauggggugguuugguuugguuuccuuuuaacuuucuuuuuguuauuugcuuuucuccuccaccugugugguauauuuuuuaagcagaauuuuauuuuuuaaaauaaaagguucuuuacaagaugauaccuuaauuacacucccgcaacacagccauuauuuuauugucuagcuccaguuaucuguauuuuauguaauguaauugacaggauggcugcugcagaaugcugguugacacagggauuauuauacugcuauuuuucccugaauucuuuuccuuggaauuccaacuguggaccuuuuauaugugccuucacuuuagcuguuugccuuacucuacagccuugcucuccggggugguuaauaaaaugcaacacuuggcauuuuuauguuauaagaaaaacaguauuuuauuuauaauaaaaucugaauauuuuguaacccuuu

SEQ ID NO: 550 - RNA, 2121 nt, Artificial Sequence
  Description of Artificial Sequence Synthetic polynucleotide
auccacuccuuccacaguaccggauucucucuuuaacccuccccuucguguuucccccaauguuuaaaauguuuggaugguuuguuguucugccuggagacaaggugcuaacauagauuuaagugaauacauuaacggugcuaaaaaugaaaauucuaacccaagacaugacauucuuagcuguaacuuaacuauuaaggccuuuuccacacgcauuaauagucccauuuuucucuugccauuuguagcuuugcccauugucuuauuggcacauggguggacacggaucugcugggcucugccuuaaacacacauugcagcuucaacuuuucucuuuaguguucuguuugaaacuaauacuuaccgagucagacuuuguguucauuucauuucagggucuuggcugccugugggcuuccccagguggccuggaggugggcaaagggaaguaacagacacacgauguugucaaggaugguuuugggacuagaggcucaguggugggagagaucccugcagaacccaccaaccagaacgugguuugccugaggcuguaacugagagaaagauucuggggcuguguuaugaaaauauagacauucucacauaagcccaguucaucaccauuuccuccuuuaccuuucagugcaguuucuuuucacauuaggcuguugguucaaacuuuugggagcacggacugucaguucucugggaaguggucagcgcauccugcagggcuucuccuccucugucuuuuggagaaccagggcucuucucaggggcucuagggacugccaggcuguuucagccaggaaggccaaaaucaagagugagauguagaaaguuguaaaauagaaaaaguggaguuggugaaucgguuguucuuuccucacauuuggaugauugucauaagguuuuuagcauguuccuccuuuucuucacccuccccuuuuuucuucuauuaaucaagagaaacuucaaaguuaaugggauggucggaucucacaggcugagaacucguucaccuccaagcauuucaugaaaaagcugcuucuuauuaaucauacaaacucucaccaugaugugaagaguuucacaaauccuucaaaauaaaaaguaaugacuuagaaacugccuuccugggugauuugcaugugucuuagucuuagucaccuuauuauccugacacaaaaacacaugagcauacaugucuacacaugacuacacaaaugcaaaccuuugcaaacacauuaugcuuuugcacacacacaccuguacacacacaccggcauguuuauacacagggaguguaugguuccuguaagcacuaaguuagcuguuuucauuuaaugaccugugguuuaacccuuuugaucacuaccaccauuaucagcaccagacugagcagcuauauccuuuuauuaaucauggucauucauucauucauucauucacaaaauauuuaugauguauuuacucugcaccaggucccaugccaagcacuggggacacaguuauggcaaaguagacaaagcauuuguucauuuggagcuuagaguccaggaggaauacauuagauaaugacacaaucaaauauaaauugcaagaugucacaggugugaugaagggagaguaggagagaccaugaguauguguaacaggaggacacagcauuauucuagugcuguacuguuccguacggcagccacuacccacauguaacuuuuuaagauuuaaauuuaaauuaguuaacauucaaaacgcagcuccccaaucacacuagcaacauuucaagugcuugagagccaugcaugauuagugguuacccuauugaauaggucagaaguagaaucuuuucaucaucacagaaaguucuauuggacagugcucuucuagaucaucauaagacuacagagcacuuuucaaagcucaugcauguucaucauguuagugucguauuuugagcugggguuuugagacuccccuuagagauagagaaacagacccaagaaaugugcucaauugcaaugggccacauaccuagaucuccagaugucauuuccccucucuuauuuuaaguuauguuaagauuacuaaaacaauaaaagcuccuaaaaaauc

SEQ ID NO: 551 - RNA, 1789 nt, Artificial Sequence
  Description of Artificial Sequence Synthetic polynucleotide
gaauggugcuucucagcucugcuuaaaugcugcaguuuuaaugcaguugucaacaaguagaaccucaguuugcuaacugaaguguuuuauuaguauuuuacucuagugguguaauuguaauguagaacaguugugugguagugugaaccguaugaaccuaaguaguuuggaagaaaaaguaggguuuuuguauacuagcuuuuguauuugaauuaauuaucauuccagcuuuuuauauacuauauuucauuuaugaagaaauugauuuucuuuugggagucacuuuuaaucuguaauuuuaaaauacaagucugaauauuuauaguugauucuuaacugugcauaaaccuagauauaccauuaucccuuuuauaccuaagaagggcaugcuaauaauuaccacugucaaagaggcaaagguguugauuuuuguauaugaaguuaagccucaguggagucucauuuguuaguuuuuagugguaacuaaggguaaacucaggguucccugagcuauaugcacacucagaccucuuugcuuuaccagugguguuugugaguugcucaguaguaaaaacuggcccuuaccugacagagcccuggcuuugaccugcucagcccuguguguuaauccucuaguagccaauuaacuacucugggguggcagguuccagagaaugcaguagaccuuuugccacucaucuguguuuuacuugagacauguaaauaugauagggaaggaacugaauuucuccauucauauuuauaaccauucuaguuuuaucuuccuuggcuuuaagagugugccauggaaagugauaagaaaugaacuucuaggcuaagcaaaaagaugcuggagauauuugauacucucauuuaaacuggugcuuuauguacaugagauguacuaaaauaaguaauauagaauuuuucuugcuagguaaauccaguaagccaauaauuuuaaagauucuuuaucugcaucauugcuguuuguuacuauaaauuaaaugaaccucauggaaagguugagguguauaccuuugugauuuucuaaugaguuuuccauggugcuacaaauaauccagacuaccaggucugguagauauuaaagcuggguacuaagaaauguuauuugcauccucucaguuacuccugaauauucugauuucauacguacccagggagcaugcuguuuugucaaucaauauaaaauauuuaugaggucucccccacccccaggagguuauaugauugcucuucucuuuauaauaagagaaacaaauucuuauugugaaucuuaacaugcuuuuuagcuguggcuaugauggauuuuauuuuuuccuaggucaagcuguguaaaagucauuuauguuauuuaaaugauguacuguacugcuguuuacauggacguuuugugcgggugcuuugaagugccuugcaucagggauuaggagcaauuaaauuauuuuuucacgggacuguguaaagcauguaacuagguauugcuuugguauauaacuauuguagcuuuacaagagauuguuuuauuugaauggggaaaauacccuuuaaauuaugacggacauccacuagagauggguuugaggauuuuccaagcguguaauaaugauguuuuuccuaacaugacagaugaguaguaaauguugauauauccuauacaugacagugugagacuuuuucauuaaauaauauugaaagauuuuaaaauucauuugaaagucugauggcuuuuacaauaaaagauauuaagaauuguuauccuuaacuu

SEQ ID NO: 552 - RNA, 5926 nt, Artificial Sequence
  Description of Artificial Sequence Synthetic polynucleotide
ucgucggagcagacgggaguuucuccucggggucggagcaggaggcacgcggagugugaggccacgcaugagcggacgcuaacccccuccccagccacaaagagucuacaugucuagggucuagacauguucagcuuuguggaccuccggcuccugcuccucuuagcggccaccgcccuccugacgcacggccaagaggaaggccaagucgagggccaagacgaagacaucccaccaaucaccugcguacagaacggccucagguaccaugaccgagacguguggaaacccgagcccugccggaucugcgucugcgacaacggcaagguguugugcgaugacgugaucugugacgagaccaagaacugccccggcgccgaaguccccgagggcgagugcugucccgucugccccgacggcucagagucacccaccgaccaagaaaccaccggcgucgagggacccaagggagacacuggcccccgaggcccaaggggacccgcaggccccccuggccgagauggcaucccuggacagccuggacuucccggaccccccggaccccccggaccucccggacccccuggccucggaggaaacuuugcuccccagcugucuuauggcuaugaugagaaaucaaccggaggaauuuccgugccuggccccaugggucccucugguccucguggucucccuggccccccuggugcaccugguccccaaggcuuccaaggucccccuggugagccuggcgagccuggagcuucaggucccauggguccccgaggucccccaggucccccuggaaagaauggagaugauggggaagcuggaaaaccuggucguccuggugagcgugggccuccugggccucagggugcucgaggauugcccggaacagcuggccucccuggaaugaagggacacagagguuucagugguuuggauggugccaagggagaugcugguccugcugguccuaagggugagccuggcagcccuggugaaaauggagcuccuggucagaugggcccccguggccugccuggugagagaggucgcccuggagccccuggcccugcuggugcucguggaaaugauggugcuacuggugcugccgggcccccuggucccaccggccccgcugguccuccuggcuucccuggugcuguuggugcuaagggugaagcugguccccaagggccccgaggcucugaagguccccagggugugcguggugagccuggccccccuggcccugcuggugcugcuggcccugcuggaaacccuggugcugauggacagccuggugcuaaaggugccaauggugcuccugguauugcuggugcuccuggcuucccuggugcccgaggccccucuggaccccagggccccggcggcccuccuggucccaaggguaacagcggugaaccuggugcuccuggcagcaaaggagacacuggugcuaagggagagccuggcccuguugguguucaaggacccccuggcccugcuggagaggaaggaaagcgaggagcucgaggugaacccggacccacuggccugcccggacccccuggcgagcgugguggaccugguagccgugguuucccuggcgcagaugguguugcuggucccaagggucccgcuggugaacgugguucuccuggcccugcuggccccaaaggaucuccuggugaagcuggucgucccggugaagcuggucugccuggugccaagggucugacuggaagcccuggcagcccugguccugauggcaaaacuggccccccuggucccgccggucaagauggucgccccggacccccaggcccaccuggugcccguggucaggcuggugugaugggauucccuggaccuaaaggugcugcuggagagcccggcaaggcuggagagcgagguguucccggacccccuggcgcugucgguccugcuggcaaagauggagaggcuggagcucagggacccccuggcccugcuggucccgcuggcgagagaggugaacaaggcccugcuggcucccccggauuccagggucucccugguccugcugguccuccaggugaagcaggcaaaccuggugaacaggguguuccuggagaccuuggcgccccuggccccucuggagcaagaggcgagagagguuucccuggcgagcguggugugcaaggucccccugguccugcugguccccgaggggccaacggugcucccggcaacgauggugcuaagggugaugcuggugccccuggagcucccgguagccagggcgccccuggccuucagggaaugccuggugaacguggugcagcuggucuuccagggccuaagggugacagaggugaugcuggucccaaaggugcugauggcucuccuggcaaagauggcguccguggucugacuggccccauugguccuccuggcccugcuggugccccuggugacaagggugaaaguggucccagcggcccugcuggucccacuggagcucguggugcccccggagaccguggugagccugguccccccggcccugcuggcuuugcuggccccccuggugcugacggccaaccuggugcuaaaggcgaaccuggugaugcuggugcuaaaggcgaugcuggucccccuggcccugccggacccgcuggacccccuggccccauugguaauguuggugcuccuggagccaaaggugcucgcggcagcgcuggucccccuggugcuacugguuucccuggugcugcuggccgagucgguccuccuggccccucuggaaaugcuggacccccuggcccuccugguccugcuggcaaagaaggcggcaaagguccccguggugagacuggcccugcuggacguccuggugaaguuggucccccuggucccccuggcccugcuggcgagaaaggauccccuggugcugaugguccugcuggugcuccugguacucccgggccucaagguauugcuggacagcgugguguggucggccugccuggucagagaggagagagaggcuucccuggucuuccuggccccucuggugaaccuggcaaacaaggucccucuggagcaaguggugaacguggucccccuggucccaugggccccccuggauuggcuggacccccuggugaaucuggacgugagggggcuccuggugccgaagguuccccuggacgagacgguucuccuggcgccaagggugaccguggugagaccggccccgcuggacccccuggugcuccuggugcuccuggugccccuggccccguuggcccugcuggcaagaguggugaucguggugagacugguccugcuggucccgccgguccugucggcccuguuggcgcccguggccccgccggaccccaaggcccccguggugacaagggugagacaggcgaacagggcgacagaggcauaaagggucaccguggcuucucuggccuccagggucccccuggcccuccuggcucuccuggugaacaaggucccucuggagccucugguccugcugguccccgaggucccccuggcucugcuggugcuccuggcaaagauggacucaacggucucccuggccccauugggcccccugguccucgcggucgcacuggugaugcugguccuguugguccccccggcccuccuggaccuccuggucccccugguccucccagcgcugguuucgacuucagcuuccugccccagccaccucaagagaaggcucacgaugguggccgcuacuaccgggcugaugaugccaaugugguucgugaccgugaccucgagguggacaccacccucaagagccugagccagcagaucgagaacauccggagcccagagggcagccgcaagaaccccgcccgcaccugccgugaccucaagaugugccacucugacuggaagaguggagaguacuggauugaccccaaccaaggcugcaaccuggaugccaucaaagucuucugcaacauggagacuggugagaccugcguguaccccacucagcccaguguggcccagaagaacugguacaucagcaagaaccccaaggacaagaggcaugucugguucggcgagagcaugaccgauggauuccaguucgaguauggcggccagggcuccgacccugccgauguggccauccagcugaccuuccugcgccugauguccaccgaggccucccagaacaucaccuaccacugcaagaacagcguggccuacauggaccagcagacuggcaaccucaagaaggcccugcuccuccagggcuccaacgagaucgagauccgcgccgagggcaacagccgcuucaccuacagcgucacugucgauggcugcacgagucacaccggagccuggggcaagacagugauugaauacaaaaccaccaagaccucccgccugcccaucaucgauguggcccccuuggacguuggugccccagaccaggaauucggcuucgacguuggcccugucugcuuccuguaaacucccuccaucccaaccuggcucccucccacccaaccaacuuuccccccaacccggaaacagacaagcaacccaaacugaacccccucaaaagccaaaaaaugggagacaauuucacauggacuuuggaaaauauuuuuuuccuuugcauucaucucucaaacuuaguuuuuaucuuugaccaaccgaacaugaccaaaaaccaaaagugcauucaaccuuaccaaaaaaaaaaaaaaaaaaagaauaaauaaauaacuuuuuaaaaaaggaagcuugguccacuugcuugaagacccaugcggggguaagucccuuucugcccguugggcuuaugaaaccccaaugcugcccuuucugcuccuuucuccacaccccccuuggggccuccccuccacuccuucccaaaucugucuccccagaagacacaggaaacaauguauugucugcccagcaaucaaaggcaaugcucaaacacccaaguggcccccacccucagcccgcuccugcccgcccagcacccccaggcccugggggaccugggguucucagacugccaaagaagccuugccaucuggcgcucccauggcucuugcaacaucuccccuucguuuuugagggggucaugccgggggagccaccagccccucacuggguucggaggagagucaggaagggccacgacaaagcagaaacaucggauuuggggaacgcgugucaaucccuugugccgcagggcugggcgggagagacuguucuguuccuuguguaacuguguugcugaaagacuaccucguucuugucuugaugugucaccggggcaacugccugggggcggggaugggggcaggguggaagcggcuccccauuuuauaccaaaggugcuacaucuaugugaugggugggguggggagggaaucacuggugcuauagaaauugagaugcccccccaggccagcaaauguuccuuuuuguucaaagucuauuuuuauuccuugauauuuuucuuuuuuuuuuuuuuuuuuuguggauggggacuugugaauuuuucuaaaggugcuauuuaacaugggaggagagcgugugcggcuccagcccagcccgcugcucacuuuccacccucucuccaccugccucuggcuucucaggccucugcucuccgaccucucuccucugaaacccuccuccacagcugcagcccauccucccggcucccuccuagucuguccugcguccucuguccccggguuucagagacaacuucccaaagcacaaagcaguuuuucccccuaggggugggaggaagcaaaagacucuguaccuauuuuguauguguauaauaauuugagauguuuuuaauuauuuugauugcuggaauaaagcauguggaaaugacccaaacaua

SEQ ID NO: 553 - RNA, 5412 nt, Artificial Sequence
  Description of Artificial Sequence Synthetic polynucleotide
gugucccauaguguuuccaaacuuggaaagggcgggggagggcgggaggaugcggagggcggagguaugcagacaacgagucagaguuuccccuugaaagccucaaaaguguccacguccucaaaaagaauggaaccaauuuaagaagccagccccguggccacgucccuucccccauucgcucccuccucugcgcccccgcaggcuccucccagcuguggcugcccgggcccccagccccagcccucccauugguggaggcccuuuuggaggcacccuagggccagggaaacuuuugccguauaaauagggcagauccgggcuuuauuauuuuagcaccacggcagcaggagguuucggcuaaguuggagguacuggccacgacugcaugcccgcgcccgccaggugauaccuccgccggugacccaggggcucugcgacacaaggagucugcaugucuaagugcuagacaugcucagcuuuguggauacgcggacuuuguugcugcuugcaguaaccuuaugccuagcaacaugccaaucuuuacaagaggaaacuguaagaaagggcccagccggagauagaggaccacguggagaaagggguccaccaggccccccaggcagagauggugaagauggucccacaggcccuccugguccaccugguccuccuggccccccuggucucggugggaacuuugcugcucaguaugauggaaaaggaguuggacuuggcccuggaccaaugggcuuaaugggaccuagaggcccaccuggugcagcuggagccccaggcccucaagguuuccaaggaccugcuggugagccuggugaaccuggucaaacugguccugcaggugcucgugguccagcuggcccuccuggcaaggcuggugaagauggucacccuggaaaacccggacgaccuggugagagaggaguuguuggaccacagggugcucgugguuucccuggaacuccuggacuuccuggcuucaaaggcauuaggggacacaauggucuggauggauugaagggacagcccggugcuccuggugugaagggugaaccuggugccccuggugaaaauggaacuccaggucaaacaggagcccgugggcuuccuggugagagaggacguguuggugccccuggcccagcuggugcccguggcagugauggaagugugggucccguggguccugcuggucccauugggucugcuggcccuccaggcuucccaggugccccuggccccaagggugaaauuggagcuguugguaacgcugguccugcuggucccgccgguccccguggugaagugggucuuccaggccucuccggccccguuggaccuccugguaauccuggagcaaacggccuuacuggugccaagggugcugcuggccuucccggcguugcuggggcucccggccucccuggaccccgcgguauuccuggcccuguuggugcugccggugcuacuggugccagaggacuuguuggugagccugguccagcuggcuccaaaggagagagcgguaacaagggugagcccggcucugcugggccccaagguccuccuggucccaguggugaagaaggaaagagaggcccuaauggggaagcuggaucugccggcccuccaggaccuccugggcugagagguaguccugguucucguggucuuccuggagcugauggcagagcuggcgucaugggcccuccugguagucguggugcaaguggcccugcuggaguccgaggaccuaauggagaugcuggucgcccuggggagccuggucucaugggacccagaggucuuccugguuccccuggaaauaucggccccgcuggaaaagaagguccugucggccucccuggcaucgacggcaggccuggcccaauuggcccagcuggagcaagaggagagccuggcaacauuggauucccuggacccaaaggccccacuggugauccuggcaaaaacggugauaaaggucaugcuggucuugcuggugcucggggugcuccagguccugauggaaacaauggugcucagggaccuccuggaccacaggguguucaagguggaaaaggugaacagggucccccugguccuccaggcuuccagggucugccuggccccucaggucccgcuggugaaguuggcaaaccaggagaaaggggucuccauggugaguuuggucucccugguccugcugguccaagaggggaacgcggucccccaggugagaguggugcugccgguccuacugguccuauuggaagccgagguccuucuggacccccagggccugauggaaacaagggugaaccuggugugguuggugcugugggcacugcugguccaucugguccuaguggacucccaggagagaggggugcugcuggcauaccuggaggcaagggagaaaagggugaaccuggucucagaggugaaauugguaacccuggcagagauggugcucguggugcuccuggugcuguaggugccccugguccugcuggagccacaggugaccggggcgaagcuggggcugcugguccugcugguccugcugguccucggggaagcccuggugaacguggugaggucgguccugcuggccccaauggauuugcugguccugcuggugcugcuggucaaccuggugcuaaaggagaaagaggagccaaagggccuaagggugaaaacgguguuguuggucccacaggccccguuggagcugcuggcccagcugguccaaaugguccccccgguccugcuggaagucguggugauggaggccccccugguaugacugguuucccuggugcugcuggacggacuggucccccaggacccucugguauuucuggcccuccuggucccccugguccugcugggaaagaagggcuucgugguccucguggugaccaagguccaguuggccgaacuggagaaguaggugcaguuggucccccuggcuucgcuggugagaagggucccucuggagaggcugguacugcuggaccuccuggcacuccagguccucagggucuucuuggugcuccugguauucugggucucccuggcucgagaggugaacguggucuaccagguguugcuggugcugugggugaaccugguccucuuggcauugccggcccuccuggggcccgugguccuccuggugcuguggguaguccuggagucaacggugcuccuggugaagcuggucgugauggcaacccugggaacgauggucccccaggucgcgauggucaacccggacacaagggagagcgcgguuacccuggcaauauuggucccguuggugcugcaggugcaccugguccucauggccccguggguccugcuggcaaacauggaaaccguggugaaacugguccuucugguccuguugguccugcuggugcuguuggcccaagagguccuaguggcccacaaggcauucguggcgauaagggagagcccggugaaaaggggcccagaggucuuccuggcuuaaagggacacaauggauugcaaggucugccugguaucgcuggucaccauggugaucaaggugcuccuggcuccguggguccugcugguccuaggggcccugcugguccuucuggcccugcuggaaaagauggucgcacuggacauccugguacaguuggaccugcuggcauucgaggcccucagggucaccaaggcccugcuggccccccuggucccccuggcccuccuggaccuccagguguaagcgguggugguuaugacuuugguuacgauggagacuucuacagggcugaccagccucgcucagcaccuucucucagacccaaggacuaugaaguugaugcuacucugaagucucucaacaaccagauugagacccuucuuacuccugaaggcucuagaaagaacccagcucgcacaugccgugacuugagacucagccacccagaguggagcagugguuacuacuggauugacccuaaccaaggaugcacuauggaugcuaucaaaguauacugugauuucucuacuggcgaaaccuguauccgggcccaaccugaaaacaucccagccaagaacugguauaggagcuccaaggacaagaaacacgucuggcuaggagaaacuaucaaugcuggcagccaguuugaauauaauguagaaggagugacuuccaaggaaauggcuacccaacuugccuucaugcgccugcuggccaacuaugccucucagaacaucaccuaccacugcaagaacagcauugcauacauggaugaggagacuggcaaccugaaaaaggcugucauucuacagggcucuaaugauguugaacuuguugcugagggcaacagcagguucacuuacacuguucuuguagauggcugcucuaaaaagacaaaugaauggggaaagacaaucauugaauacaaaacaaauaagccaucacgccugcccuuccuugauauugcaccuuuggacaucgguggugcugaccaggaauucuuuguggacauuggcccagucuguuucaaauaaaugaacucaaucuaaauuaaaaaagaaagaaauuugaaaaaacuuucucuuugccauuucuucuucuucuuuuuuaacugaaagcugaauccuuccauuucuucugcacaucuacuugcuuaaauugugggcaaaagagaaaaagaaggauugaucagagcauugugcaauacaguuucauuaacuccuucccccgcucccccaaaaauuugaauuuuuuuuucaacacucuuacaccuguuauggaaaaugucaaccuuuguaagaaaaccaaaauaaaaauugaaaaauaaaaaccauaaacauuugcaccacuuguggcuuuugaauaucuuccacagagggaaguuuaaaacccaaacuuccaaagguuuaaacuaccucaaaacacuuucccaugagugugauccacauuguuaggugcugaccuagacagagaugaacugagguccuuguuuuguuuuguucauaauacaaaggugcuaauuaauaguauuucagauacuugaagaauguugauggugcuagaagaauuugagaagaaauacuccuguauugaguuguaucgugugguguauuuuuuaaaaaauuugauuuagcauucauauuuuccaucuuauucccaauuaaaaguaugcagauuauuugcccaaaucuucuucagauucagcauuuguucuuugccagucucauuuucaucuucuuccaugguuccacagaagcuuuguuucuugggcaagcagaaaaauuaaauuguaccuauuuuguauaugugagauguuuaaauaaauugugaaaaaaaugaaauaaagcauguuugguuuuccaaaagaacauaua

SEQ ID NO: 554 - RNA, 5491 nt, Artificial Sequence
  Description of Artificial Sequence Synthetic polynucleotide
ggcugaguuuuaugacgggcccggugcugaagggcagggaacaacuugauggugcuacuuugaacugcuuuucuuuucuccuuuuugcacaaagagucucaugucugauauuuagacaugaugagcuuugugcaaaaggggagcuggcuacuucucgcucugcuucaucccacuauuauuuuggcacaacaggaagcuguugaaggaggauguucccaucuuggucaguccuaugcggauagagaugucuggaagccagaaccaugccaaauaugugucugugacucaggauccguucucugcgaugacauaauaugugacgaucaagaauuagacugccccaacccagaaauuccauuuggagaauguugugcaguuugcccacagccuccaacugcuccuacucgcccuccuaauggucaaggaccucaaggccccaagggagauccaggcccuccugguauuccugggagaaauggugacccugguauuccaggacaaccaggguccccugguucuccuggccccccuggaaucugugaaucaugcccuacugguccucagaacuauucuccccaguaugauucauaugaugucaagucuggaguagcaguaggaggacucgcaggcuauccuggaccagcuggccccccaggcccucccggucccccugguacaucuggucauccugguuccccuggaucuccaggauaccaaggacccccuggugaaccugggcaagcugguccuucaggcccuccaggaccuccuggugcuauagguccaucugguccugcuggaaaagauggagaaucagguagacccggacgaccuggagagcgaggauugccuggaccuccagguaucaaagguccagcugggauaccuggauucccugguaugaaaggacacagaggcuucgauggacgaaauggagaaaagggugaaacaggugcuccuggauuaaagggugaaaauggucuuccaggcgaaaauggagcuccuggacccauggguccaagaggggcuccuggugagcgaggacggccaggacuuccuggggcugcaggugcucgggguaaugacggugcucgaggcagugauggucaaccaggcccuccugguccuccuggaacugccggauucccuggauccccuggugcuaagggugaaguuggaccugcagggucuccugguucaaauggugccccuggacaaagaggagaaccuggaccucagggacacgcuggugcucaagguccuccuggcccuccugggauuaaugguaguccuggugguaaaggcgaaaugggucccgcuggcauuccuggagcuccuggacugaugggagcccgggguccuccaggaccagccggugcuaauggugcuccuggacugcgagguggugcaggugagccugguaagaauggugccaaaggagagcccggaccacguggugaacgcggugaggcugguauuccagguguuccaggagcuaaaggcgaagauggcaaggauggaucaccuggagaaccuggugcaaaugggcuuccaggagcugcaggagaaaggggugccccuggguuccgaggaccugcuggaccaaauggcaucccaggagaaaaggguccugcuggagagcguggugcuccaggcccugcagggcccagaggagcugcuggagaaccuggcagagauggcgucccuggagguccaggaaugaggggcaugcccggaaguccaggaggaccaggaagugaugggaaaccagggccucccggaagucaaggagaaaguggucgaccagguccuccugggccaucugguccccgaggucagccuggugucaugggcuuccccgguccuaaaggaaaugauggugcuccugguaagaauggagaacgagguggcccuggaggaccuggcccucaggguccuccuggaaagaauggugaaacuggaccucagggacccccagggccuacugggccugguggugacaaaggagacacaggacccccugguccacaaggauuacaaggcuugccugguacaggugguccuccaggagaaaauggaaaaccuggggaaccagguccaaagggugaugccggugcaccuggagcuccaggaggcaagggugaugcuggugccccuggugaacguggaccuccuggauuggcaggggccccaggacuuagagguggagcuggucccccuggucccgaaggaggaaagggugcugcugguccuccugggccaccuggugcugcugguacuccuggucugcaaggaaugccuggagaaagaggaggucuuggaaguccugguccaaagggugacaagggugaaccaggcgguccaggugcugauggugucccagggaaagauggcccaagggguccuacugguccuauugguccuccuggcccagcuggccagccuggagauaagggugaagguggugcccccggacuuccagguauagcuggaccucgugguagcccuggugagagaggugaaacuggcccuccaggaccugcugguuucccuggugcuccuggacagaauggugaaccuggugguaaaggagaaagaggggcuccgggugagaaaggugaaggaggcccuccuggaguugcaggacccccuggagguucuggaccugcugguccuccugguccccaaggugucaaaggugaacguggcaguccugguggaccuggugcugcuggcuucccuggugcucguggucuuccugguccuccugguaguaaugguaacccaggacccccaggucccagcgguucuccaggcaaggaugggcccccagguccugcggguaacacuggugcuccuggcagcccuggagugucuggaccaaaaggugaugcuggccaaccaggagagaagggaucgccuggugcccagggcccaccaggagcuccaggcccacuugggauugcugggaucacuggagcacggggucuugcaggaccaccaggcaugccagguccuaggggaagcccuggcccucagggugucaagggugaaagugggaaaccaggagcuaacggucucaguggagaacguggucccccuggaccccagggucuuccuggucuggcugguacagcuggugaaccuggaagagauggaaacccuggaucagauggucuuccaggccgagauggaucuccugguggcaagggugaucguggugaaaauggcucuccuggugccccuggcgcuccuggucauccaggcccaccugguccugucgguccagcuggaaagaguggugacagaggagaaaguggcccugcuggcccugcuggugcucccgguccugcugguucccgaggugcuccugguccucaaggcccacguggugacaaaggugaaacaggugaacguggagcugcuggcaucaaaggacaucgaggauucccugguaauccaggugccccagguucuccaggcccugcuggucagcagggugcaaucggcaguccaggaccugcaggccccagaggaccuguuggacccaguggaccuccuggcaaagauggaaccaguggacauccaggucccauuggaccaccagggccucgagguaacagaggugaaagaggaucugagggcuccccaggccacccagggcaaccaggcccuccuggaccuccuggugccccugguccuugcuguggugguguuggagccgcugccauugcugggauuggaggugaaaaagcuggcgguuuugccccguauuauggagaugaaccaauggauuucaaaaucaacaccgaugagauuaugacuucacucaagucuguuaauggacaaauagaaagccucauuaguccugaugguucucguaaaaaccccgcuagaaacugcagagaccugaaauucugccauccugaacucaagaguggagaauacuggguugacccuaaccaaggaugcaaauuggaugcuaucaagguauucuguaauauggaaacuggggaaacaugcauaagugccaauccuuugaauguuccacggaaacacugguggacagauucuagugcugagaagaaacacguuugguuuggagaguccauggauggugguuuucaguuuagcuacggcaauccugaacuuccugaagauguccuugaugugcagcuggcauuccuucgacuucucuccagccgagcuucccagaacaucacauaucacugcaaaaauagcauugcauacauggaucaggccaguggaaauguaaagaaggcccugaagcugauggggucaaaugaaggugaauucaaggcugaaggaaauagcaaauucaccuacacaguucuggaggaugguugcacgaaacacacuggggaauggagcaaaacagucuuugaauaucgaacacgcaaggcugugagacuaccuauuguagauauugcacccuaugacauuggugguccugaucaagaauuugguguggacguuggcccuguuugcuuuuuauaaaccaaacucuaucugaaaucccaacaaaaaaaauuuaacuccauauguguuccucuuguucuaaucuugucaaccagugcaagugaccgacaaaauuccaguuauuuauuuccaaaauguuuggaaacaguauaauuugacaaagaaaaaugauacuucucuuuuuuugcuguuccaccaaauacaauucaaaugcuuuuuguuuuauuuuuuuaccaauuccaauuucaaaaugucucaauggugcuauaauaaauaaacuucaacacucuuuaugauaacaacacuguguuauauucuuugaauccuagcccaucugcagagcaaugacugugcucaccaguaaaagauaaccuuucuuucugaaauagucaaauacgaaauuagaaaagcccucccuauuuuaacuaccucaacuggucagaaacacagauuguauucuaugagucccagaagaugaaaaaaauuuuauacguugauaaaacuuauaaauuucauugauuaaucuccuggaagauugguuuaaaaagaaaaguguaaugcaagaauuuaaagaaauauuuuuaaagccacaauuauuuuaauauuggauaucaacugcuuguaaaggugcuccucuuuuuucuugucauugcuggucaagauuacuaauauuugggaaggcuuuaaagacgcauguuauggugcuaauguacuuucacuuuuaaacucuagaucagaauuguugacuugcauucagaacauaaaugcacaaaaucuguacaugucucccaucagaaagauucauuggcaugccacaggggauucuccuccuucauccuguaaaggucaacaauaaaaaccaaauuauggggcugcuuuugucacacuagcauagagaauguguugaaauuuaacuuuguaagcuuguaugugguuguugaucuuuuuuuuccuuacagacacccauaauaaaauaucauauuaaaauuca

SEQ ID NO: 555 - RNA, 6494 nt, Artificial Sequence
  Description of Artificial Sequence Synthetic polynucleotide
gaaguccggccuuccgagagcuagcuguccgccgcggcccccgcacgccgggcagccgucccucgccgccucgggcgcgccaccauggggccccggcucagcgucuggcugcugcugcugcccgccgcccuucugcuccacgaggagcacagccgggccgcugcgaaggguggcugugcuggcucuggcuguggcaaaugugacugccauggagugaagggacaaaagggugaaagaggccucccgggguuacaaggugucauuggguuuccuggaaugcaaggaccugaggggccacagggaccaccaggacaaaagggugauacuggagaaccaggacuaccuggaacaaaagggacaagaggaccuccgggagcaucuggcuacccuggaaacccaggacuucccggaauuccuggccaagacggcccgccaggccccccagguauuccaggaugcaauggcacaaagggggagagagggccgcucgggccuccuggcuugccugguuucgcuggaaaucccggaccaccaggcuuaccagggaugaagggugauccaggugagauacuuggccaugugcccgggaugcuguugaaaggugaaagaggauuucccggaaucccagggacuccaggcccaccaggacugccagggcuucaagguccuguugggccuccaggauuuaccggaccaccaggucccccaggcccucccggcccuccaggugaaaagggacaaaugggcuuaaguuuucaaggaccaaaaggugacaagggugaccaaggggucagugggccuccaggaguaccaggacaagcucaaguucaagaaaaaggagacuucgccaccaagggagaaaagggccaaaaaggugaaccuggauuucaggggaugccaggggucggagagaaaggugaacccggaaaaccaggacccagaggcaaacccggaaaagauggugacaaaggggaaaaagggagucccgguuuuccuggugaacccggguacccaggacucauaggccgccagggcccgcagggagaaaagggugaagcagguccuccuggcccaccuggaauuguuauaggcacaggaccuuugggagaaaaaggagagaggggcuacccuggaacuccggggccaagaggagagccaggcccaaaagguuucccaggacuaccaggccaacccggaccuccaggccucccuguaccugggcaggcuggugccccuggcuucccuggugaaagaggagaaaaaggugaccgaggauuuccugguacaucucugccaggaccaaguggaagagaugggcucccggguccuccugguuccccugggcccccugggcagccuggcuacacaaauggaauuguggaaugucagcccggaccuccaggugaccaggguccuccuggaauuccagggcagccaggauuuauaggcgaaauuggagagaaaggucaaaaaggagagaguugccucaucugugauauagacggauaucgggggccucccgggccacagggacccccgggagaaauagguuucccagggcagccaggggccaagggcgacagagguuugccuggcagagaugguguugcaggagugccaggcccucaagguacaccagggcugauaggccagccaggagccaagggggagccuggugaguuuuauuucgacuugcggcucaaaggugacaaaggagacccaggcuuuccaggacagcccggcaugccagggagagcggguucuccuggaagagauggccauccgggucuuccuggccccaagggcucgccggguucuguaggauugaaaggagagcguggccccccuggaggaguuggauucccaggcagucguggugacaccggccccccugggccuccaggauaugguccugcuggucccauuggugacaaaggacaagcaggcuuuccuggaggcccuggauccccaggccugccagguccaaagggugaaccaggaaaaauuguuccuuuaccaggccccccuggagcagaaggacugccgggguccccaggcuucccagguccccaaggagaccgaggcuuucccggaaccccaggaaggccaggccugccaggagagaagggcgcugugggccagccaggcauuggauuuccagggccccccggccccaaagguguugacggcuuaccuggagacauggggccaccggggacuccaggucgcccgggauuuaauggcuuaccugggaacccaggugugcagggccagaagggagagccuggaguuggucuaccgggacucaaagguuugccaggucuucccggcauuccuggcacacccggggagaaggggagcauugggguaccaggcguuccuggagaacauggagcgaucggacccccugggcuucaggggaucagaggugaaccgggaccuccuggauugccaggcuccguggggucuccaggaguuccaggaauaggccccccuggagcuaggggucccccuggaggacagggaccaccgggguugucaggcccuccuggaauaaaaggagagaaggguuuccccggauucccuggacuggacaugccgggcccuaaaggagauaaaggggcucaaggacucccuggcauaacgggacagucggggcucccuggccuuccuggacagcagggggcuccugggauuccuggguuuccagguuccaagggagaaaugggcgucauggggacccccgggcagccgggcucaccaggaccagugggugcuccuggauuaccgggugaaaaaggggaccauggcuuuccgggcuccucaggacccaggggagacccuggcuugaaaggugauaagggggaugucggucucccuggcaagccuggcuccauggauaagguggacaugggcagcaugaagggccagaaaggagaccaaggagagaaaggacaaauuggaccaauuggugagaagggaucccgaggagacccugggaccccaggagugccuggaaaggacgggcaggcaggacagccugggcagccaggaccuaaaggugauccagguauaaguggaaccccaggugcuccaggacuuccgggaccaaaaggaucuguugguggaaugggcuugccaggaacaccuggagagaaaggugugccuggcaucccuggcccacaagguucaccuggcuuaccuggagacaaaggugcaaaaggagagaaagggcaggcaggcccaccuggcauaggcaucccagggcugcgaggugaaaagggagaucaagggauagcggguuucccaggaagcccuggagagaagggagaaaaaggaagcauugggaucccaggaaugccaggguccccaggccuuaaagggucucccgggaguguuggcuauccaggaaguccugggcuaccuggagaaaaaggugacaaaggccucccaggauuggauggcaucccuggugucaaaggagaagcaggucuuccugggacuccuggccccacaggcccagcuggccagaaaggggagccaggcagugauggaaucccggggucagcaggagagaagggugaaccaggucuaccaggaagaggauucccaggguuuccaggggccaaaggagacaaagguucaaagggugagguggguuucccaggauuagccgggagcccaggaauuccuggauccaaaggagagcaaggauucauggguccuccggggccccagggacagccgggguuaccgggauccccaggccaugccacggaggggcccaaaggagaccgcggaccucagggccagccuggccugccaggacuuccgggacccauggggccuccagggcuuccugggauugauggaguuaaaggugacaaaggaaauccaggcuggccaggagcacccggugucccagggcccaagggagacccuggauuccagggcaugccugguauugguggcucuccaggaaucacaggcucuaagggugauauggggccuccaggaguuccaggauuucaagguccaaaaggucuuccuggccuccagggaauuaaaggugaucaaggcgaucaaggcgucccgggagcuaaaggucucccggguccuccuggccccccagguccuuacgacaucaucaaaggggagcccgggcucccugguccugagggccccccagggcugaaagggcuucagggacugccaggcccgaaaggccagcaagguguuacaggauugguggguauaccuggaccuccagguauuccuggguuugacggugccccuggccagaaaggagagaugggaccugccgggccuacugguccaagaggauuuccagguccaccaggccccgauggguugccaggauccauggggcccccaggcaccccaucuguugaucacggcuuccuugugaccaggcauagucaaacaauagaugacccacaguguccuucugggaccaaaauucuuuaccacggguacucuuugcucuacgugcaaggcaaugaacgggcccauggccaggacuugggcacggccggcagcugccugcgcaaguucagcacaaugcccuuccuguucugcaauauuaacaacgugugcaacuuugcaucacgaaaugacuacucguacuggcuguccaccccugagcccaugcccaugucaauggcacccaucacgggggaaaacauaagaccauuuauuaguaggugugcugugugugaggcgccugccauggugauggccgugcacagccagaccauucagaucccaccgugccccagcgggugguccucgcuguggaucggcuacucuuuugugaugcacaccagcgcuggugcagaaggcucuggccaagcccuggcgucccccggcuccugccuggaggaguuuagaagugcgccauucaucgagugucacggccgugggaccugcaauuacuacgcaaacgcuuacagcuuuuggcucgccaccauagagaggagcgagauguucaagaagccuacgccguccaccuugaaggcaggggagcugcgcacgcacgucagccgcugccaagucuguaugagaagaacauaaugaagccugacucagcuaaugucacaacauggugcuacuucuucuucuuuuuguuaacagcaacgaacccuagaaauauauccuguguaccucacuguccaauaugaaaaccguaaagugccuuauaggaauuugcguaacuaacacacccugcuucauugaccucuacuugcugaaggagaaaaagacagcgauaagcuuucaauaguggcauaccaaauggcacuuuugaugaaauaaaauaucaauauuuucugcaauccaaugcacugaugugugaagugagaacuccaucagaaaaccaaagggugcuaggaggugugggugccuuccauacuguuugcccauuuucauucuuguauuauaauuaauuuucuacccccagagauaaauguuuguuuauaucacugucuagcuguuucaaaauuuaggucccuuggucuguacaaauaauagcaauguaaaaaugguuuuuugaaccuccaaauggaauuacagacucaguagccauaucuuccaaccccccaguauaaauuucugucuuucugcuaugugugguacuuugcagcugcuuuugcagaaaucacaauuuuccuguggaauaaagaugguccaaaaauagucaaaaauuaaauauauauauauauuaguaauuuauauagaugucagcaauuaggcagaucaagguuuaguuuaacuuccacuguuaaaauaaagcuuacauaguuuucuuccuuugaaagacugugcuguccuuuaacauagguuuuuaaagacuaggauauugaaugugaaacauccguuuucauuguucacuucuaaaccaaaaauuauguguugccaaaaccaaacccagguucaugaauauggugucuauuauagugaaacauguacuuugagcuuauuguuuuuauucuguauuaaauauuuucaggguuuuaaacacuaaucacaaacugaaugacuugacuucaaaagcaacaaccuuaaaggccgucauuucauuaguauuccucauucugcauccuggcuugaaaaacagcucuguugaaucacaguaucaguauuuucacacguaagcacauucgggccauuuccgugguuucucaugagcuguguucacagaccucagcagggcaucgcauggaccgcaggagggcagauucggaccacuaggccugaaaugacauuucacuaaaagucuccaaaacauuucuaagacuacuaaggccuuuuauguaauuucuuuaaauguguauuucuuaagaauucaaauuuguaauaaaacuauuuguauaaaaauuaagcuuuuauuaauuuguugcuaguauugccacagacgcauuaaaagaaacuuacugcacaagcugcuaauaaauuuguaagcuuugcauaccuua

SEQ ID NO: 556 - RNA, 6262 nt, Artificial Sequence
  Description of Artificial Sequence Synthetic polynucleotide
gaguguggcugcagugcgccgggacaccagggcuccgcgcuccgcacucaagaggcucccgcgucccaaccccucgcgcccgcgcguucgcggauccaggccgaggaccgaaaggggccgcccgagcccccggggccggcgcccagagagcccagcaaggccggccgcccugccggugugccgccggcgggugcuucuggaagggccaaugcguucgggcagcagccccugaagccgagcccgaggcuaagugggacugaccggggcccagaguggacgaaccgccagcauggggagagaccagcgcgcgguggccggcccugcccuacggcgguggcugcugcuggggacagugaccgugggguuccucgcccagagcgucuuggcgggugugaagaaguuugaugugccguguggaggaagagauugcagugggggcugccagugcuacccugagaaagguggacguggucagccugggccagugggcccccagggguacaaugggccaccaggauuacaaggauucccgggacugcagggacguaaaggagacaagggugaaaggggagcccccggaguaacgggacccaagggcgacgugggagcaagaggcguuucuggauucccuggugccgauggaauuccuggacacccggggcaaggugggcccaggggaaggccgggcuacgauggcugcaacggaacccagggagacucagguccacaggggccccccggcucugagggguucaccgggccucccgggccccaaggaccaaaagggcagaaaggugagccuuaugcacugccuaaagaggagcgcgacagauaucggggugaaccuggagagccuggauuggucgguuuccagggaccucccggccgcccugggcaugugggacagauggguccaguuggagcuccagggagaccaggaccaccuggacccccuggaccaaaaggacagcaaggcaacagaggacuugguuucuacggaguuaagggugaaaagggugacguagggcagccgggacccaacgggauuccaucagacacccuccaccccaucaucgcgcccacaggagucaccuuccacccagaucaguacaagggugaaaaaggcagugagggggaaccaggaauaagaggcauuuccuugaagggagaagaaggaaucaugggcuuuccuggacugagggguuacccuggcuugaguggugaaaaaggaucaccaggacagaagggaagccgaggccuggauggcuaucaagggccugauggaccccggggacccaagggagaagccggagacccagggcccccuggacuaccugccuacuccccucacccuucccuagcaaaaggugccagaggugacccgggauucccaggggcccaaggggagccaggaagccagggugagccaggagacccgggccucccaggucccccuggccucuccaucggagauggagaucagaggagaggccugccgggugagaugggacccaagggcuucaucggagaccccggcaucccugcgcucuacgggggcccaccuggaccugauggaaagcgagggccuccaggaccccccgggcucccuggaccaccuggaccugauggcuuccuguuugggcugaaaggagcaaaaggaagagcaggcuucccugggcuucccggcuccccuggagcccgcggaccaaagggguggaaaggugacgcuggggaaugcagauguacagaaggcgacgaagcuaucaaaggucuuccgggacugccaggacccaagggcuucgcaggcaucaacggggagccggggaggaaaggggacagaggagaccccggccaacacggccucccuggguucccagggcucaagggagugccuggcaacauuggugcucccggacccaaaggagcaaaaggagauuccagaacaaucacaaccaaaggugagcggggacagcccggcgucccaggugugcccgggaugaaaggugacgauggcagcccaggccgcgaugggcucgauggauuccccggccucccaggcccucccggugauggcaucaagggcccuccaggggacccaggcuauccaggaauaccuggaacgaaggguacuccaggagaaaugggccccccaggacugggccuucccggccucaaaggccaacgugguuucccuggagacgccggcuuaccuggaccaccaggcuuccugggcccuccuggccccgcagggaccccaggacaaauagauugugacacagaugugaaaagggccguuggaggugacagacaggaggccauccagccagguugcauaggagggcccaagggauugccaggccugccaggacccccaggccccacaggugccaaaggccuccgaggaaucccaggcuucgcaggagcugauggaggaccagggcccaggggcuugccaggagacgcaggucgugaaggguucccaggacccccaggguucauaggaccccgaggauccaaaggugcagugggccucccuggcccagauggauccccaggucccaucggccugccagggccagaugggcccccuggggaaaggggccucccuggagaaguccugggagcucagcccgggccacggggagaugcuggugugccuggacagccugggcuuaaaggccuucccggagacagaggccccccuggauucagaggaagccaagggaugccugggaugccagggcugaagggccagccaggccucccaggaccuuccggccagccaggccuguaugggccuccaggacugcauggauucccaggagcuccuggccaagaggggcccuuggggcugccaggaaucccaggccgugaaggucugccuggugauagaggggacccuggggacacaggcgcuccuggcccugugggcaugaaaggucucucuggugacagaggagaugcuggcuucacaggggagcaaggccauccaggaagcccuggauuuaaaggaauugauggaaugccugggacccccgggcuaaaaggagauagaggcucaccugggauggaugguuuccaaggcaugccuggacucaaagggagacccggguuuccagggagcaaaggcgaggcuggauuuuucggaauacccggucugaagggucuggcuggugagccagguuuuaaaggcagccgaggggacccugggcccccaggaccaccuccugucauccugccaggaaugaaagacauuaaaggagagaaaggagaugaagggccuauggggcugaaaggauaccugggcgcaaaagguauccaaggaaugccaggcaucccagggcugucaggaaucccugggcugccugggaggcccggccacaucaaaggagucaagggagacaucggaguccccggcauccccgguuugccaggauucccugggguggcuggccccccuggaauuacgggauucccaggauucauaggaagccggggugacaaaggugccccagggagagcaggccuguauggcgagauuggcgcgacuggugauuucggugacaucggggacacuauaaauuuaccaggaagaccaggccugaagggggagcggggcaccacuggaauaccaggucugaagggauucuuuggagagaagggaacagaaggugacaucggcuucccugggauaacaggcgugacuggaguccaaggcccuccuggacuuaaaggacaaacaggcuuuccagggcugacugggccuccagggucgcagggagagcuggggcggauuggacugccugguggcaaaggagaugauggcuggccgggagcuccgggcuuaccagguuuuccgggacuccgugggauccgcggcuuacacggcuugccaggcaccaagggcuuuccaggauccccagguucugacauccacggagacccaggcuucccaggcccuccuggggaaagaggugacccaggagaggccaacacccuuccaggcccugugggagucccaggacagaaaggagaccaaggagcuccaggggaacgaggcccaccugggagcccaggacuucagggguucccaggcaucacacccccuuccaacaucucuggggcaccuggugacaaaggggcgccagggauauuuggccugaaagguuaucggggcccaccagggccaccagguucugcugcucuuccuggaagcaaaggugacacagggaacccaggagcuccaggaaccccagggaccaaaggaugggccggggacuccgggccccagggcaggccugguguguuuggucucccaggagaaaaagggcccaggggugaacaaggcuucauggggaacacuggacccaccggggcggugggcgacagaggccccaagggacccaagggagacccaggauucccuggugcccccgggacugugggagcccccgggauugcaggaaucccccagaagauugccguccaaccagggacaguggguccccaggggaggcgaggccccccuggggcaccgggggagauggggccccagggcccccccggagaaccagguuuucguggggcuccagggaaagcugggccccaaggaagagguggugugucugcuguucccggcuuccggggagaugaaggacccauaggccaccaggggccgauuggccaagaaggugcaccaggccguccagggagcccgggccugccggguaugccaggccgcagcgucagcaucggcuaccuccuggugaagcacagccagacggaccaggagcccaugugcccagugggcaugaacaaacucuggaguggauacagccugcuguacuucgagggccaggagaaggcgcacaaccaggaccuggggcuggcgggcuccugccuggcgcgguucagcaccaugcccuuccuguacugcaacccuggugaugucugcuacuaugccagccggaacgacaaguccuacuggcucucuaccacugcgccgcugcccaugaugcccguggccgaggacgagaucaagcccuacaucagccgcuguucugugugugaggccccggccaucgccaucgcgguccacagucaggaugucuccaucccacacugcccagcuggguggcggaguuuguggaucggauauuccuuccucaugcacacggcggcgggagacgaaggcgguggccaaucacuggugucaccgggcagcugucuagaggacuuccgcgccacaccauucaucgaaugcaauggaggccgcggcaccugccacuacuacgccaacaaguacagcuucuggcugaccaccauucccgagcagagcuuccagggcucgcccuccgccgacacgcucaaggccggccucauccgcacacacaucagccgcugccaggugugcaugaagaaccugugagccggcgcgugccaggaagggccauuuuggugcuuauucuuaacuuauuaccucaggugccaacccaaaaauugguuuuauuuuuuucuuaaaaaaaaaaaagucuaccaaaggaauuugcauccagcagcagcacuuagaccugccagccacugucaccgagcgggugcaagcacucggggucccuggagggcaagcccugcccacagaaagccaggagcagcccuggcccccaucagcccugcuagacgcaccgccugaaggcacagcuaaccacuucgcacacacccauguaaccacugcacuuuccaaugccacagacaacucacauuguucaacucccuucucggggugggacagacgagacaacagcacacaggcagccagccguggccagaggcucgaggggcucagggccucaggcacccguccccacacgagggccccgugggugggccuggcccugcuuucuacgccaauguuaugccagcuccauguucucccaaauaccguugaugugaauuauuuuaaaggcaaaaccgugcucuuuauuuuaaaaaacacugauaaucacacugcgguaggucauucuuuugccacaucccuauagaccacuggguuuggcaaaacucaggcagaaguggagaccuuucuagacaucauugucagccuugcuacuugaagguacaccccauagggucggaggugcuguccccacugccccacguugucccugagauuuaaccccuccacugcugggggugagcuguacucuucugacugcccccuccuguguaacgacuacaaaauaaaacuugguucugaauauuuuua

SEQ ID NO: 557 - RNA, 5274 nt, Artificial Sequence
  Description of Artificial Sequence Synthetic polynucleotide
ggagucggguuucagagcgcgggugacucggggcgcgggccgggagccgggauucugcccgccgccgccgcugccgagcgccgccuuuguucccugcaggaagggcgagcgcggcggccagcgcucagcgacccuucguccuccgcuaagcuccaacgcucugcucgacuagccgcgcgccuuccggggcuccgcagacccgcgagauggcaccaaggaggaacaacgggcagugcuggugucugcugaugcugcucucggucuccacgccccucccugcugucacccagacccgcggugcgacagagacugcuucccagggucaccuggaccucacgcagcucaucggugucccgcugcccucguccguauccuuugucacaggcuaugguggcuucccggccuacaguuucgggccuggugccaauguuggccgcccagccaggacucucaucccauccaccuucuucagggacuucgccaucagcgucguggugaagcccagcagcacccgugguggcgugcucuucgccaucacugacgccuuccagaaggucaucuaccugggccugcggcucucagguguggaggacggccaccagcggaucauccucuacuacacggagccaggcucccaugugucccaagaggcugcugccuucucggugccugugaugacccacagguggaaccgcuucgccaugauuguccagggugaggaagugacccuccucgugaacugugaggagcacagccgcauccccuuccagcgguccucccaggcuuuggcuuuugaguccagcgcuggaaucuucaugggcaaugcaggagcuacagggcucgagagauucacuggcucccuccagcagcucaccgugcaccccgaccccaggacucccgaggagcugugugacccugaagaguccucggcaucuggagagaccagugggcugcaggaggcagacggaguagcugagaucuuagaagccgucaccuacacucaagccucgcccaaagaagcaaaaguugaacccauaaacacaccuccaacuccauccucccccuuugaagacauggaacuuucuggugaaccuguacccgaggggacccuggaaaccaccaacaugagcaucauccagcacagcagccccaaacaagggucuggugagauccugaaugacacacuggaggggguucauucuguggauggugaccccauuacugacagcggcucaggggcuggggccuuccuugacauugcugaagaaaagaauuuagcagcaacagcagcggggcuggccgaggugcccaucagcacugcuggagaagcagaggccagcagugugcccaccgggggaccaacccucucuauguccacggagaacccagaggaaggggucacuccagguccagauaaugaagagcguuuagcagcaacagcagcaggggaggccgaggcacucgccagcaugccuggggaaguggaggccagugguguggcccccggggagcuggaccucuccauguccgcccagagccucggggaagaggccacuguggguccaagcagugaagacaguuuaacaacagcugcagcugcaaccgaagugucccucaguacuuuugaggaugaggaagccagugggguccccacagauggccuggcuccccucacagccaccauggccccugagcgggcagucacuucugguccuggugaugaagaagacuuggcagcagccacaacagaggagccccucaucacagcugggggugaagaguccggcagcccucccccugaugggccaccgcugccccugcccacaguggcuccugaaagauggaucacuccagcucaaagagaacaugugggaaugaaaggacaggcugggcccaaaggagaaaagggugaugcuggggaggagcuuccuggcccuccugaaccuucugggccuguuggacccacggcaggagcagaagcagagggcucuggccuaggcuggggcucggacgucggcucuggcucuggugaccuggugggcagugagcagcugcugagagguccuccaggacccccagggccaccuggcuuaccugggauuccaggaaaaccaggaacugauguuuucaugggacccccuggaucuccuggagaggauggaccugcuggugaaccugggcccccgggcccugagggacagccuggaguugauggagccaccggccuucccgggaugaaaggggagaagggagcaagagggccuaauggcucaguuggugaaaagggugacccuggcaacagaggcuuaccuggacccccggggaaaaagggacaagcuggcccuccuggggucaugggacccccagggccuccuggacccccugggcccccaggcccuggaugcacaaugggacuuggauucgaggauaccgaaggcucuggaagcacccagcuauugaaugaacccaaacucuccagaccaacggcugcaauuggucucaaaggagagaaaggagaccggggacccaagggagaaagggggauggauggagccaguauugugggacccccugggccgagagggccaccugggcacaucaaggucuugucuaauuccuugaucaauaucacccauggauucaugaauuucucggacauuccugagcugguggggccuccggggccggacggguugccugggcugccaggauuuccagguccuagaggaccaaaaggugacacugguuuaccuggcuuuccaggacuaaaaggagaacagggcgagaagggagagccgggugccauccugacagaggacauuccucuggaaaggcugauggggaaaaagggugaaccuggaaugcauggagccccaggaccaauggggcccaaaggaccaccaggacauaaaggagaauuuggccuucccgggcgaccuggucgcccaggacugaauggccucaaggguaccaaaggagauccaggggucauuaugcagggcccaccuggcuuaccuggcccuccaggccccccugggccaccuggagcugugauuaacaucaaaggagccauuuucccaauacccguccgaccacacugcaaaaugccaguugauacugcucauccugggaguccagagcucaucacuuuucacgguguuaaaggagagaaaggauccuggggucuuccuggcucaaagggagaaaaaggcgaccagggagcccagggaccaccagguccuccacuugaucuagcuuaccugagacacuuucugaacaacuugaagggggagaauggagacaagggguucaaaggugaaaaaggagaaaaaggagacauuaauggcagcuuccuuaugucugggccuccaggccugcccggaaauccaggcccggcuggccaaaaaggggagacagucguugggccccaaggacccccaggugcuccuggucugccugggccaccuggcuuuggaagaccuggugauccugggccaccggggcccccggggccaccaggaccuccagcuauccugggagcagcuguggcccuuccaggucccccuggcccuccaggacagccagggcuucccggauccagaaaccuggucacagcauucagcaacauggaugacaugcugcagaaagcgcauuugguuauagaaggaacauucaucuaccugagggacagcacugaguuuuucauucguguuagagauggcuggaaaaaauuacagcugggagaacugauccccauuccugccgacagcccuccacccccugcgcuuuccagcaacccacaucagcuucugccuccaccaaacccuauuucaagugccaauuaugagaagccugcucugcauuuggcugcucugaacaugccauuuucuggggacauucgagcugauuuucagugcuucaagcaggccagagcugcaggacuguuguccaccuaccgagcauucuuaucuucccauuugcaagaucuguccaccauugugaggaaagcagagagauacagccuucccauagugaaccucaagggccaaguacuuuuuaauaauugggacucaauuuuuucuggccacggaggucaguucaauaugcauauuccaauauacuccuuugauggucgagacauaaugacagauccuucuuggccccagaaagucauuuggcauggcuccagcccccauggcguccgccuuguggauaacuacugugaagcauggcgaaccgcggacacagcggucacgggacuugccuccccgcugagcacggggaagauucuggaccagaaagcauacagcugugcuaaucggcuaauuguccuauguaucgaaaacaguuucaugacagacgcuaggaaguaauggccuucugaugauucuuaaagaguuuucaauuuuuucuuaugugaagaguugacacugaaaucuaaaauguuuaauuguuguaaauauuacaguuuuuuuuuuuuacuacauauucuuuacaacagcaaccaaagaaaacauaccucaauacacucaaaacugaagacauagaggacucagaucaaagacaaaaucugauccauauauuggugcuagauucugcaggaaaccccagcagugugaacgcaucccaacauagguuaagagcaaguugaaaacaaaggccauggcauucugccacugcauccuucagacaguuauauccuccuuuuaaaccauuguuguugaguguaagauguccuucauguuuucuuauaaagucaguguuuagaaauguuacccuuucuaaguuauauacagaucaaaugcuuuuuucuuucacguacauccaucauuugcaacugcuguucguacacagaaacaggacugcucaaaugauccuauuuguauuuucugaugcuaucagacucuaauguuuuuuucccuaaaauauuauugccaucaugcuuuaggaauuuuauauuuuuacacaaucauauuuuaguauggugucuguuuauguaacucugacuugcuggaaaaguugaaacuccaaauaaucugaaacuagaaaagaaauagcacauaauuacuaccuuccccuuggcggcucuccuccccaacccccaccccacaauuuuaugacuuccauuuggcaauuguugaauuauaacugcgacugaaacaaacagguucauagagaugaauuuucugagaaacauauaucuacauguuguauaauuggauuuuuuuuccauguaagugaacauaaaaacaucuuuuccgggugcuuucuuca

SEQ ID NO: 558 - RNA, 1843 nt, Artificial Sequence
  Description of Artificial Sequence Synthetic polynucleotide
gucugaggcucggccgccugagccgcggacgguuugcugagcccguuagugcgcccggccgagacacgccgccgccaugucccgcuaccugcguccccccaacacgucucuguucgucaggaacguggccgacgacaccaggucugaagacuugcggcgugaauuuggucguuaugguccuauaguugauguguauguuccacuugauuucuacacucgccguccaagaggauuugcuuauguucaauuugaggauguucgugaugcugaagacgcuuuacauaauuuggacagaaaguggauuuguggacggcagauugaaauacaguuugcccagggggaucgaaagacaccaaaucagaugaaagccaaggaagggaggaauguguacaguucuucacgcuaugaugauuaugacagauacagacguucuagaagccgaaguuaugaaaggaggagaucaagaagucggucuuuugauuacaacuauagaagaucguauaguccuagaaacaguagaccgacuggaagaccacggcguagcagaagccauuccgacaaugauagaccaaacugcagcuggaauacccaguacaguucugcuuacuacacuucaagaaagaucugaaagcggaaaaagaaccaaagaagggcaguucaagcgaccaaaggguggguggaaggugcugcaguaugaauacuguacgaauauuuugacucuggucugaaaagauaaaagaauguuaucgaaaacuacauggaauaauugaagucccuucaaguuugaaaguaagcauuuuaggacaaauaaaaggaaauucaacuuuguacuuguggaaacuaaucccuaaauaugaauagguuuauauugauucauggguaacagguccauaauaaauuauuggaaacuaggaugucugaauaucaaggaagacagccauagucucuuacagugccucuguuggucugucucaaacugaauugggugggaaaagguaugguccaauauaaaaguuccauuuuugccauuauuggcaaaucuugccuuuguuuauuuuggugccaguguuuucugcuuaaucauuugcuuuguuggcaucuguguuuauuuacuuguacaccacaugcaguuuacaucugucuuaacuacuccuucccagguaaauuccaauuauauuugacauccagcuaagagggcccaucucuucucaccucuuuccuagucaguauauucagcaaauauuuauugagcccuuacugugggcaaaucauuguacuggauaauugagaaaaauagauaauucccuuauucaguaaaugucuacugagcacaaucuagugaaucauuacaguauggccucauuguuuuguuugagguguguuauucauaacaauauuuuacaccauucguaucaauguaauuauagaacacaauauacgaucaaggauaaguaauugugugguuaucugccauuuaaaaguauccaguauuugaucacauuauuauaaauaaugaaaaaaugauuuaaucuguaauaaacugguuuauugugcagugacuguaauauacuagaguuauaauaaauuguuuacucugccucaccaaacacaugcuaggauauaacccccaaaauaaguauuuaacuuugcauuagguauaaaggagacugggugcuauaauuagauuauuuugaggcagacagagagcuguuauccuaacugauuuaguauguucuguaauugagaaaauguucaccaaauuauacuuuuuagugauuuacauguacauuuuauaggggacauguucuguguauagcgaauaaauaacuuuuauaguaucaca

SEQ ID NO: 559 - RNA, 2925 nt, Artificial Sequence
  Description of Artificial Sequence Synthetic polynucleotide
gucugaggcucggccgccugagccgcggacgguuugcugagcccguuagugcgcccggccgagacacgccgccgccaugucccgcuaccugcguccccccaacacgucucuguucgucaggaacguggccgacgacaccaggucugaagacuugcggcgugaauuuggucguuaugguccuauaguugauguguauguuccacuugauuucuacacucgccguccaagaggauuugcuuauguucaauuugaggauguucgugaugcugaagacgcuuuacauaauuuggacagaaaguggauuuguggacggcagauugaaauacaguuugcccagggggaucgaaagacaccaaaucagaugaaagccaaggaagggaggaauguguacaguucuucacgcuaugaugauuaugacagauacagacguucuagaagccgaaguuaugaaaggaggagaucaagaagucggucuuuugauuacaacuauagaagaucguauaguccuagaaacaguagaccgacuggaagaccacggcguagcagaagccauuccgacaaugauagauucaaacaccgaaaucgaucuuuuucaagaucuaaauccaauucaagaucacgguccaagucccagcccaagaaagaaaugaaggcuaaaucacguucuaggucugcaucucacaccaaaacuagaggcaccucuaaaacagauuccaaaacacauuauaagucuggcucaagauaugaaaaggaaucaaggaaaaaagaaccaccuagauccaaaucucagucaagaucacagucuaggucuaggucaaaaucuagaucaaggucuuggacuaguccuaaguccaguggccacugauaguauaaaccauggucauuuuuaggcauguaucauucauuuacucauaguuugguuuacuuaaauuaucaggaauacaauguugcaaugaugcuuaaaaaacacuuguuaguuuucccuguaccaggcaaugguuauaauuaaaaugauaugcuguugagaagccacucuuaagaguccaguuuguuuaauguuaugggcagcuaccaauuuguggugucucuguauauuuuuguaaagauucucauuuuuuaugcuugaaguauuuggugaaaagauguugguugaccauaauuugcaacauugucucauuaaaaauaaacuuucauauucauauuugguagaacuguuaaccuagaaauguagcuugcuaauaagauagaaugauacaaaagugaaguaguagccacaguacaacacugacugcucagacacauuuagguucaggguggaccuuuaugucuugucaagaugucuaggcccggcugggcgugguggcucacaccuguaaucccagcacuuugggaggccgaggcgggcggaucacgaggucaggaguucgagaccagccugaccaacacggugaaaccccgucucuacuaaaaauacaaaaauuauccgggcaugguggcacaugccuguaaucucagcuacucaggaggcugaggcaagagaaucgcuugaaccugggagguagaaguugcagugagccaaaaucacgccacugcacuccagccugggcaacagagugagacuccgucucaaaaaaaaaaaaaaaccggaugucuaggccaaugauaauuauuuuugaugcaguguggauuaguucuuuuguuaaccccacugucuuggggaaugaugccagcugggaaauugaguuuuugacugaaacauggagccuucacugcuuuuuuucugguuccuaugaagauuuggaacauagaaaacacaaaaacucaccuuaaaauuugagcaggucguugauggcaaaaauaauuuuaaggaaaaaggaauauucuuauguaguuauucuaaaguuuaaggagcguuguugaccauaauauugcuuaguuuucuuacugcuguuaaguaaguaaauuguuucaaaguagguuuugugugugugugccuaguguaaaagaacugaaauuuugaugcuuacagcacuuggcucgugcauuuguaucaaaauuugccugccucuuuaugagggaggccugcuuuucacaccucaguuuauuuaauacgaggcaaguuguaagacaacacucauucuaggugauucuguggugccaugaaauuuaagguaauuuggggaaaaggauuagucaguuuuaagcaagagucacaucuuuugagcuuucgauuaucaguguaguaccugacuaaaaaugaaguaauacccuuaaaccauuuauaauuucuaguauuucucugaaagaucguuuuggggacaaaagugacuugacauguccaauuucauuucagaauaaaaagcuagcaucuuuaaaaaucucagauugcuugcuuacagauacaaguacgaauuauggacaaacgauuccuuuuagaggauuacuuuuuucaauuucgguuuuaguaaucuaggcuuugccuguaaagaauacaacgauggauuuuaaauacuguuuguggaauguguuuaaaggauugauucuagaaccuuuguauauuugauaguauuucuaacuuucauuucuuuacuguuugcaguuaauguucauguucugcuaugcaaucguuuauaugcacguuucuuuaauuuuuuuagauuuuccuggauguauaguuuaaacaacaaaaagucuauuuaaaacuguagcaguaguuuacaguucuagcaaagaggaaaguugugggguuaaacuuuguauuuucuuucuuauagaggcuucuaaaaagguauuuuuauauguucuuuuuaacaaauauuguguacaaccuuuaaaacaucaauguuuggaucaaaacaagacccagcuuauuuucugcuugcuguaaauuaagcaaacaugcuauaauaaaaacaaaaugaagga

SEQ ID NO: 560 - RNA, 1311 nt, Artificial Sequence
  Description of Artificial Sequence Synthetic polynucleotide
aaauugagcccgcagccucccgcuucgcucucugcuccuccuguucgacagucagccgcaucuucuuuugcgucgccagccgagccacaucgcucagacaccauggggaaggugaaggucggagucaacggauuuggucguauugggcgccuggucaccagggcugcuuuuaacucugguaaaguggauauuguugccaucaaugaccccuucauugaccucaacuacaugguuuacauguuccaauaugauuccacccauggcaaauuccauggcaccgucaaggcugagaacgggaagcuugucaucaauggaaaucccaucaccaucuuccaggagcgagaucccuccaaaaucaaguggggcgaugcuggcgcugaguacgucguggaguccacuggcgucuucaccaccauggagaaggcuggggcucauuugcaggggggagccaaaagggucaucaucucugcccccucugcugaugcccccauguucgucaugggugugaaccaugagaaguaugacaacagccucaagaucaucagcaaugccuccugcaccaccaacugcuuagcaccccuggccaaggucauccaugacaacuuugguaucguggaaggacucaugaccacaguccaugccaucacugccacccagaagacuguggauggccccuccgggaaacuguggcgugauggccgcggggcucuccagaacaucaucccugccucuacuggcgcugccaaggcugugggcaaggucaucccugagcugaacgggaagcucacuggcauggccuuccguguccccacugccaacgugucagugguggaccugaccugccgucuagaaaaaccugccaaauaugaugacaucaagaagguggugaagcaggcgucggagggcccccucaagggcauccugggcuacacugagcaccagguggucuccucugacuucaacagcgacacccacuccuccaccuuugacgcuggggcuggcauugcccucaacgaccacuuugucaagcucauuuccugguaugacaacgaauuuggcuacagcaacagggugguggaccucauggcccacauggccuccaaggaguaagaccccuggaccaccagccccagcaagagcacaagaggaagagagagacccucacugcuggggagucccugccacacucagucccccaccacacugaaucuccccuccucacaguugccauguagaccccuugaagaggggaggggccuagggagccgcaccuugucauguaccaucaauaaaguacccugugcucaacca

SEQ ID NO: 561 - RNA, 7923 nt, Artificial Sequence
  Description of Artificial Sequence Synthetic polynucleotide
gcgcacucgggcacgcgcucggaagucgggggucggcgcggagugcaggcugcucccgggguaggugagggaagcgcggaggcggggcgcgggggcaguggucggcgagcagcgcgguccucgcuaggggcgcccacccgucagucucuccggcgcgagccgccgccaccgcccgcgccggagucaggccccugggcccccaggcucaagcagcgaagcggccuccgggggacgccgcuaggcgagaggaacgcgccggugcccuugccuucgccgugacccagcgugcgggcggcgggaugagagggagccaucgggccgcgccggcccugcggccccgggggcggcucuggcccgugcuggccgugcuggcggcggccgccgcggcgggcugugcccaggcagccauggacgagugcacggacgagggcgggcggccgcagcgcugcaugcccgaguucgucaacgccgcuuucaacgugacugugguggccaccaacacgugugggacuccgcccgaggaauacugugugcagaccggggugaccggggucaccaaguccugucaccugugcgacgccgggcagccccaccugcagcacggggcagccuuccugaccgacuacaacaaccaggccgacaccaccugguggcaaagccagaccaugcuggccggggugcaguaccccagcuccaucaaccucacgcugcaccugggaaaagcuuuugacaucaccuaugugcgucucaaguuccacaccagccgcccggagagcuuugccauuuacaagcgcacacgggaagacgggcccuggauuccuuaccaguacuacagugguuccugcgagaacaccuacuccaaggcaaaccgcggcuucaucaggacaggaggggacgagcagcaggccuuguguacugaugaauucagugacauuucuccccucacugggggcaacguggccuuuucuacccuggaaggaaggcccagcgccuauaacuuugacaauagcccugugcugcaggaauggguaacugccacugacaucagaguaacucuuaaucgccugaacacuuuuggagaugaaguguuuaacgaucccaaaguucucaaguccuauuauuaugccaucucugauuuugcuguagguggcagauguaaauguaauggacacgcaagcgaguguaugaagaacgaauuugauaagcugguguguaauugcaaacauaacacauauggaguagacugugaaaagugucuuccuuucuucaaugaccggccguggaggagggcaacugcggaaagugccagugaaugccugcccugugauugcaauggucgaucccaggaaugcuacuucgacccugaacucuaucguuccacuggccaugggggccacuguaccaacugccaggauaacacagauggcgcccacugugagaggugccgagagaacuucuuccgccuuggcaacaaugaagccugcucuucaugccacuguaguccugugggcucucuaagcacacagugugauaguuacggcagaugcagcuguaagccaggagugaugggggacaaaugugaccguugccagccuggauuccauucucucacugaagcaggaugcaggccaugcucuugugaucccucuggcagcauagaugaauguaauguugaaacaggaagauguguuugcaaagacaaugucgaaggcuucaauugugaaagaugcaaaccuggauuuuuuaaucuggaaucaucuaauccucgggguugcacacccugcuucugcuuugggcauucuucugucuguacaaacgcuguuggcuacaguguuuauucuaucuccucuaccuuucagauugaugaggauggguggcgugcggaacagagagauggcucugaagcaucucucgagugguccucugagaggcaagauaucgccgugaucucagacagcuacuuuccucgguacuucauugcuccugcaaaguucuugggcaagcagguguugaguuauggucagaaccucuccuucuccuuucgaguggacaggcgagauacucgccucucugccgaagaccuugugcuugagggagcuggcuuaagaguaucuguacccuugaucgcucagggcaauuccuauccaagugagaccacugugaaguaugucuucaggcuccaugaagcaacagauuacccuuggaggccugcucuuaccccuuuugaauuucagaagcuccuaaacaacuugaccucuaucaagauacgugggacauacagugagagaagugcuggauauuuggaugaugucacccuggcaagugcucguccugggccuggagucccugcaacuuggguggaguccugcaccuguccugugggauauggagggcaguuuugugagaugugccucucagguuacagaagagaaacuccuaaucuuggaccauacaguccaugugugcuuugcgccugcaauggacacagcgagaccugugauccugagacagguguuuguaacugcagagacaauacggcuggcccgcacugugagaagugcagugauggguacuauggagauucaacugcaggcaccuccuccgauugccaacccuguccguguccuggagguucaaguugugcuguuguucccaagacaaaggagguggugugcaccaacuguccuacuggcaccacugguaagagaugugagcucugugaugauggcuacuuuggagacccccuggguagaaacggcccugugagacuuugccgccugugccagugcagugacaacaucgaucccaacgcaguuggaaauugcaaucgcuugacgggagaaugccugaagugcaucuauaacacugcuggcuucuauugugaccggugcaaagacggauuuuuuggaaauccccuggcucccaauccagcagacaaaugcaaagccugcaauugcaauccguaugggaccaugaagcagcagagcagcuguaaccccgugacggggcagugugaauguuugccucacgugacuggccaggacuguggugcuugugacccuggauucuacaaucugcagagugggcaaggcugugagaggugugacugccaugccuugggcuccaccaaugggcagugugacauccgcaccggccagugugagugccagcccggcaucacuggucagcacugugagcgcugugaggucaaccacuuuggguuuggaccugaaggcugcaaacccugugacugucauccugagggaucucuuucacuucagugcaaagaugauggucgcugugaaugcagagaaggcuuugugggaaaucgcugugaccagugugaagaaaacuauuucuacaaucggucuuggccuggcugccaggaauguccagcuuguuaccggcugguaaaggauaagguugcugaucauagagugaagcuccaggaauuagagagucucauagcaaaccuuggaacuggggaugagauggugacagaucaagccuucgaggauagacuaaaggaagcagagagggaaguuauggaccuccuucgugaggcccaggaugucaaagauguugaccagaauuugauggaucgccuacagagagugaauaacacucuguccagccaaauuagccguuuacagaauauccggaauaccauugaagagacuggaaacuuggcugaacaagcgcgugcccauguagagaacacagagcgguugauugaaaucgcauccagagaacuugagaaagcaaaagucgcugcugccaaugugucagucacucagccagaaucuacaggggacccaaacaacaugacucuuuuggcagaagaggcucgaaagcuugcugaacgucauaaacaggaagcugaugacauuguucgaguggcaaagacagccaaugauacgucaacugaggcauacaaccugcuucugaggacacuggcaggagaaaaucaaacagcauuugagauugaagagcuuaauaggaaguaugaacaagcgaagaacaucucacaggaucuggaaaaacaagcugcccgaguacaugaggaggccaaaagggccggugacaaagcuguggagaucuaugccagcguggcucagcugagcccuuuggacucugagacacuggagaaugaagcaaauaacauaaagauggaagcugagaaucuggaacaacugauugaccagaaauuaaaagauuaugaggaccucagagaagauaugagagggaaggaacuugaagucaagaaccuucuggagaaaggcaagacugaacagcagaccgcagaccaacuccuagcccgagcugaugcugccaaggcccucgcugaagaagcugcaaagaagggacgggauaccuuacaagaagcuaaugacauucucaacaaccugaaagauuuugauaggcgcgugaacgauaacaagacggccgcagaggaggcacuaaggaagauuccugccaucaaccagaccaucacugaagccaaugaaaagaccagagaagcccagcaggcccugggcagugcugcggcggaugccacagaggccaagaacaaggcccaugaggcggagaggaucgcaagcgcuguccaaaagaaugccaccagcaccaaggcagaagcugaaagaacuuuugcagaaguuacagaucuggauaaugaggugaacaauauguugaagcaacugcaggaagcagaaaaagagcuaaagagaaaacaagaugacgcugaccaggacaugaugauggcagggauggcuucacaggcugcucaagaagccgagaucaaugccagaaaagccaaaaacucuguuacuagccuccucagcauuauuaaugaccucuuggagcagcuggggcagcuggauacaguggaccugaauaagcuaaacgagauugaaggcacccuaaacaaagccaaagaugaaaugaaggucagcgaucuugauaggaaagugucugaccuggagaaugaagccaagaagcaggaggcugccaucauggacuauaaccgagauaucgaggagaucaugaaggacauucgcaaucuggaggacaucaggaagaccuuaccaucuggcugcuucaacaccccguccauugaaaagcccuagugucuuuagggcuggaaggcagcaucccucugacaggggggcaguugugaggccacagagugccuugacacaaagauuacauuuuucagacccccacuccucugcugcuguccaucacuguccuuuugaaccaggaaaagucacagaguuuaaagagaagcaaauuaaacauccugaaucgggaacaaaggguuuuaucuaauaaagugucucuuccaucacguugcuaccuuacccacacuucccucugauuugcgugaggacguggcauccuacuuacguacguggcauaacacaucgugugagcccauguaugcugggguagagcaaguagcccuccccugucucaucgauccagcagaaccuccucagucucaguacucuuguuucuauaaggaaaaguuuugcuacuaacaguagcauugugauggccaguauauccaguccauggauaaagaaaaugcaucugcaucuccugccccucuuccuucuaagcaaaaggaaauaaacauccugugccaaagguauuggucauuuagaaugucgguagccauccaucagugcuuuuagcuauuaugaguguaggacacugagccauccgugggucaggaugcaauuauuuauaaaaguccccaggugaacauggcugaagauuuuucuaguauauuaauaauugacuaggaagaugaacuuuuuuucagaucuuugggcagcugauaauuuaaaucuggaugggcagcuugcacucaccaauagaccaaaagacaucuuuugauauucuuauaaauggaacuuacacagaagaaauagggauaugauaaccacuaaaguuuuguuuucaaaaucaaacuaauucuuacagcuuuuuuauuaguuagucuuggaacuaguguuaaguaucuggcagagaacaguuaaucccuaaggucuugacaaaacagaagaaaaacaagccuccucguccuagucuuuucuagcaaagggauaaaacuuagauggcagcuuguacugucagaaucccguguauccauuuguucuucuguuggagagaugagacauuugacccuuagcuccaguuuucuucugauguuuccaucuuccagaaucccucaaaaaacauuguuugccaaauccugguggcaaauacuugcacucaguauuucacacagcugccaacgcuaucgaguuccugcacuuugugauuuaaauccacucuaaaccuucccucuaaguguagagggaagacccuuacguggaguuuccuagugggcuucucaacuuuugauccucagcucugugguuuuaagaccacagugugacaguucccugccacacacccccuuccuccuaccaacccaccuuugagauucauauauagccuuuaacacuaugcaacuuuguacuuugcguagcaggggcugggguggggggaaagaaaccuauuaucauggacacacuggugcuauuaauuauuucaaauuuauauuuuugugugaauguuuuguguuuuguuuauccaugcuauagaacaaggaauuuauguagauauacuuaguccuauuucuagaaugacacucuguucacuuugcucaauuuuuccucuucacuggcacaaguaucugaauaccuccuucccucccuucuagaguucuuuggauuguacuccaaagaauugugccuuguguuugcagcaucuccauucucuaaauuaauauaauugcuuuccuccacacccagccacguaaagagguaacuuggguccucuuccauugcaguccugaugauccuaaccugcagcacggugguuuuacaauguuccagagcaggaacgccagguugacaagcuaugguaggauuaggaaaguuugcugaagaggaucuuugacgccacagugggacuagccaggaaugagggagaaaugcccuuuuuggcaauuguuggagcuggauagguaaguuuuauaagggaguacauuuugacugagcacuuagggcaucaggaacagugcuacuuacugguggguagacugggagaggugguguaacuuaguucuugaugaucccacuuccuguuuccaucugcuugggauauaccagaguuuaccacaaguguuuugacgauauacuccugagcuuucacucugcuggcuucucccaggccucuucuacuauggcaggagauguggugugcuguugcaaaguuuucacgucaucguuuccuggcuaguucauuucauuaaguggcuacauccuaacauaugcauuggucaagguugcagcaagaggacugaagauugacugccaagcuaguuugggugaaguucacuccagcaagucucaggccacaauggggugguuugguuugguuuccuuuuaacuuucuuuuuguuauuugcuuuucuccuccaccugugugguauauuuuuuaagcagaauuuuauuuuuuaaaauaaaagguucuuuacaagaugauaccuuaauuacacucccgcaacacagccauuauuuuauugucuagcuccaguuaucuguauuuuauguaauguaauugacaggauggcugcugcagaaugcugguugacacagggauuauuauacugcuauuuuucccugaauucuuuuccuuggaauuccaacuguggaccuuuuauaugugccuucacuuuagcuguuugccuuacucuacagccuugcucuccggggugguuaauaaaaugcaacacuuggcauuuuuauguuauaagaaaaacaguauuuuauuuauaauaaaaucugaauauuuuguaacccuuua

SEQ ID NO: 562 - RNA, 3139 nt, Artificial Sequence
  Description of Artificial Sequence Synthetic polynucleotide
guugccugucucuaaaccccuccacauucccgcgguccuucagacugcccggagagcgcgcucugccugccgccugccugccugccacugaggguucccagcaccaugagggccuggaucuucuuucuccuuugccuggccgggagggccuuggcagccccucagcaagaagcccugccugaugagacagaggugguggaagaaacuguggcagaggugacugagguaucugugggagcuaauccuguccagguggaaguaggagaauuugaugauggugcagaggaaaccgaagaggaggugguggcggaaaaucccugccagaaccaccacugcaaacacggcaaggugugcgagcuggaugagaacaacacccccaugugcgugugccaggaccccaccagcugcccagcccccauuggcgaguuugagaaggugugcagcaaugacaacaagaccuucgacucuuccugccacuucuuugccacaaagugcacccuggagggcaccaagaagggccacaagcuccaccuggacuacaucgggccuugcaaauacauccccccuugccuggacucugagcugaccgaauucccccugcgcaugcgggacuggcucaagaacguccuggucacccuguaugagagggaugaggacaacaaccuucugacugagaagcagaagcugcgggugaagaagauccaugagaaugagaagcgccuggaggcaggagaccaccccguggagcugcuggcccgggacuucgagaagaacuauaacauguacaucuucccuguacacuggcaguucggccagcuggaccagcaccccauugacggguaccucucccacaccgagcuggcuccacugcgugcuccccucauccccauggagcauugcaccacccgcuuuuucgagaccugugaccuggacaaugacaaguacaucgcccuggaugagugggccggcugcuucggcaucaagcagaaggauaucgacaaggaucuugugaucuaaauccacuccuuccacaguaccggauucucucuuuaacccuccccuucguguuucccccaauguuuaaaauguuuggaugguuuguuguucugccuggagacaaggugcuaacauagauuuaagugaauacauuaacggugcuaaaaaugaaaauucuaacccaagacaugacauucuuagcuguaacuuaacuauuaaggccuuuuccacacgcauuaauagucccauuuuucucuugccauuuguagcuuugcccauugucuuauuggcacauggguggacacggaucugcugggcucugccuuaaacacacauugcagcuucaacuuuucucuuuaguguucuguuugaaacuaauacuuaccgagucagacuuuguguucauuucauuucagggucuuggcugccugugggcuuccccagguggccuggaggugggcaaagggaaguaacagacacacgauguugucaaggaugguuuugggacuagaggcucaguggugggagagaucccugcagaacccaccaaccagaacgugguuugccugaggcuguaacugagagaaagauucuggggcuguguuaugaaaauauagacauucucacauaagcccaguucaucaccauuuccuccuuuaccuuucagugcaguuucuuuucacauuaggcuguugguucaaacuuuugggagcacggacugucaguucucugggaaguggucagcgcauccugcagggcuucuccuccucugucuuuuggagaaccagggcucuucucaggggcucuagggacugccaggcuguuucagccaggaaggccaaaaucaagagugagauguagaaaguuguaaaauagaaaaaguggaguuggugaaucgguuguucuuuccucacauuuggaugauugucauaagguuuuuagcauguuccuccuuuucuucacccuccccuuuuuucuucuauuaaucaagagaaacuucaaaguuaaugggauggucggaucucacaggcugagaacucguucaccuccaagcauuucaugaaaaagcugcuucuuauuaaucauacaaacucucaccaugaugugaagaguuucacaaauccuucaaaauaaaaaguaaugacuuagaaacugccuuccugggugauuugcaugugucuuagucuuagucaccuuauuauccugacacaaaaacacaugagcauacaugucuacacaugacuacacaaaugcaaaccuuugcaaacacauuaugcuuuugcacacacacaccuguacacacacaccggcauguuuauacacagggaguguaugguuccuguaagcacuaaguuagcuguuuucauuuaaugaccugugguuuaacccuuuugaucacuaccaccauuaucagcaccagacugagcagcuauauccuuuuauuaaucauggucauucauucauucauucauucacaaaauauuuaugauguauuuacucugcaccaggucccaugccaagcacuggggacacaguuauggcaaaguagacaaagcauuuguucauuuggagcuuagaguccaggaggaauacauuagauaaugacacaaucaaauauaaauugcaagaugucacaggugugaugaagggagaguaggagagaccaugaguauguguaacaggaggacacagcauuauucuagugcuguacuguuccguacggcagccacuacccacauguaacuuuuuaagauuuaaauuuaaauuaguuaacauucaaaacgcagcuccccaaucacacuagcaacauuucaagugcuugagagccaugcaugauuagugguuacccuauugaauaggucagaaguagaaucuuuucaucaucacagaaaguucuauuggacagugcucuucuagaucaucauaagacuacagagcacuuuucaaagcucaugcauguucaucauguuagugucguauuuugagcugggguuuugagacuccccuuagagauagagaaacagacccaagaaaugugcucaauugcaaugggccacauaccuagaucuccagaugucauuuccccucucuuauuuuaaguuauguuaagauuacuaaaacaauaaaagcuccuaaaaaauca

SEQ ID NO: 563 - RNA, 3222 nt, Artificial Sequence
  Description of Artificial Sequence Synthetic polynucleotide
ugcagcacagaaaggggguccgugggggacgguagaagccuggaggaggagcuugaguccagccacugucuggguacugccagccaucgggcccaggucucugggguugucuuaccgcagugaguaccacgcgguacuacagagaccggcugcccgugugcccggcagguggagccgcccgcaucagcggccucggggaauggaagcggagaacgcgggcagcuauucccuucagcaagcucaagcuuuuuauacguuuccauuucaacaacugauggcugaagcuccuaauauggcaguugugaaugaacagcaaaugccagaagaaguuccagccccagcuccugcucaggaaccagugcaagaggcuccaaaaggaagaaaaagaaaacccagaacaacagaaccaaaacaaccaguggaacccaaaaaaccuguugagucaaaaaaaucuggcaagucugcaaaaucaaaagaaaaacaagaaaaaauuacagacacauuuaaaguaaaaagaaaaguagaccguuuuaaugguguuucagaagcugaacuucugaccaagacucuccccgauauuuugaccuucaaucuggacauugucauuauuggcauaaacccgggacuaauggcugcuuacaaagggcaucauuacccuggaccuggaaaccauuuuuggaaguguuuguuuaugucagggcucagugagguccagcugaaccauauggaugaucacacucuaccagggaaguaugguauuggauuuaccaacaugguggaaaggaccacgcccggcagcaaagaucucuccaguaaagaauuucgugaaggaggacguauucuaguacagaaauuacagaaauaucagccacgaauagcaguguuuaauggaaaauguauuuaugaaauuuuuaguaaagaaguuuuuggaguaaagguuaagaacuuggaauuugggcuucagccccauaagauuccagacacagaaacucucugcuauguuaugccaucauccagugcaagaugugcucaguuuccucgagcccaagacaaaguucauuacuacauaaaacugaaggacuuaagagaucaguugaaaggcauugaacgaaauauggacguucaagaggugcaauauacauuugaccuacagcuugcccaagaggaugcaaagaagauggcuguuaaggaagaaaaauaugauccagguuaugaggcagcauaugguggugcuuacggagaaaauccaugcagcagugaaccuuguggcuucucuucaaaugggcuaauugagagcguggaguuaagaggagaaucagcuuucaguggcauuccuaaugggcaguggaugacccagucauuuacagaccaaauuccuuccuuuaguaaucacuguggaacacaagaacaggaagaagaaagccaugcuuaagaauggugcuucucagcucugcuuaaaugcugcaguuuuaaugcaguugucaacaaguagaaccucaguuugcuaacugaaguguuuuauuaguauuuuacucuagugguguaauuguaauguagaacaguugugugguagugugaaccguaugaaccuaaguaguuuggaagaaaaaguaggguuuuuguauacuagcuuuuguauuugaauuaauuaucauuccagcuuuuuauauacuauauuucauuuaugaagaaauugauuuucuuuugggagucacuuuuaaucuguaauuuuaaaauacaagucugaauauuuauaguugauucuuaacugugcauaaaccuagauauaccauuaucccuuuuauaccuaagaagggcaugcuaauaauuaccacugucaaagaggcaaagguguugauuuuuguauaugaaguuaagccucaguggagucucauuuguuaguuuuuagugguaacuaaggguaaacucaggguucccugagcuauaugcacacucagaccucuuugcuuuaccagugguguuugugaguugcucaguaguaaaaacuggcccuuaccugacagagcccuggcuuugaccugcucagcccuguguguuaauccucuaguagccaauuaacuacucugggguggcagguuccagagaaugcaguagaccuuuugccacucaucuguguuuuacuugagacauguaaauaugauagggaaggaacugaauuucuccauucauauuuauaaccauucuaguuuuaucuuccuuggcuuuaagagugugccauggaaagugauaagaaaugaacuucuaggcuaagcaaaaagaugcuggagauauuugauacucucauuuaaacuggugcuuuauguacaugagauguacuaaaauaaguaauauagaauuuuucuugcuagguaaauccaguaagccaauaauuuuaaagauucuuuaucugcaucauugcuguuuguuacuauaaauuaaaugaaccucauggaaagguugagguguauaccuuugugauuuucuaaugaguuuuccauggugcuacaaauaauccagacuaccaggucugguagauauuaaagcuggguacuaagaaauguuauuugcauccucucaguuacuccugaauauucugauuucauacguacccagggagcaugcuguuuugucaaucaauauaaaauauuuaugaggucucccccacccccaggagguuauaugauugcucuucucuuuauaauaagagaaacaaauucuuauugugaaucuuaacaugcuuuuuagcuguggcuaugauggauuuuauuuuuuccuaggucaagcuguguaaaagucauuuauguuauuuaaaugauguacuguacugcuguuuacauggacguuuugugcgggugcuuugaagugccuugcaucagggauuaggagcaauuaaauuauuuuuucacgggacuguguaaagcauguaacuagguauugcuuugguauauaacuauuguagcuuuacaagagauuguuuuauuugaauggggaaaauacccuuuaaauuaugacggacauccacuagagauggguuugaggauuuuccaagcguguaauaaugauguuuuuccuaacaugacagaugaguaguaaauguugauauauccuauacaugacagugugagacuuuuucauuaaauaauauugaaagauuuuaaaauucauuugaaagucugauggcuuuuacaauaaaagauauuaagaauuguuauccuuaacuua

SEQ ID NO: 564 - RNA, 23 nt, Unknown
  Description of Unknown Organism Unknown mRNA sequence
cauuuuauaccaaaggugcuaca

SEQ ID NO: 565 - RNA, 23 nt, Unknown
  Description of Unknown Organism Unknown mRNA sequence
cauuuuauacguauccacgagga

SEQ ID NO: 566 - RNA, 24 nt, Unknown
  Description of Unknown Organism Unknown mRNA sequence
ggggagggaaucacuggugcuaua

SEQ ID NO: 567 - RNA, 24 nt, Unknown
  Description of Unknown Organism Unknown mRNA sequence
ggggagggaaucacaccucgagua

SEQ ID NO: 568 - RNA, 23 nt, Unknown
  Description of Unknown Organism Unknown mRNA sequence
ugaauuuuucuaaaggugcuauu

SEQ ID NO: 569 - RNA, 23 nt, Unknown
  Description of Unknown Organism Unknown mRNA sequence
ugaauuuuucugggccccgauuu

SEQ ID NO: 570 - RNA, 22 nt, Unknown
  Description of Unknown Organism Unknown mRNA sequence
aaaaugucucaauggugcuaua

SEQ ID NO: 571 - RNA, 22 nt, Unknown
  Description of Unknown Organism Unknown mRNA sequence
aaaaugucucaaucuacguaua

SEQ ID NO: 572 - RNA, 23 nt, Unknown
  Description of Unknown Organism Unknown mRNA sequence
caacugcuuguaaaggugcuccu

SEQ ID NO: 573 - RNA, 23 nt, Unknown
  Description of Unknown Organism Unknown mRNA sequence
caacugcuuguaaacgacguccu

SEQ ID NO: 574 - RNA, 24 nt, Unknown
  Description of Unknown Organism Unknown mRNA sequence
aaagacgcauguuauggugcuaau

SEQ ID NO: 575 - RNA, 24 nt, Unknown
  Description of Unknown Organism Unknown mRNA sequence
aaagacgcauguuaucuacguaau

SEQ ID NO: 576 - RNA, 24 nt, Unknown
  Description of Unknown Organism Unknown mRNA sequence
aggaagggccauuuuggugcuuau

SEQ ID NO: 577 - RNA, 24 nt, Unknown
  Description of Unknown Organism Unknown mRNA sequence
aggaagggccauuuucaucgauau

SEQ ID NO: 578 - RNA, 23 nt, Unknown
  Description of Unknown Organism Unknown mRNA sequence
cccauagggucggaggugcuguc

SEQ ID NO: 579 - RNA, 23 nt, Unknown
  Description of Unknown Organism Unknown mRNA sequence
cccauagggucggaucaugaguc

(19) United States
(12) Patent Application Publication
(10) Pub. No.: US 2018/0230546 A1
(43) Pub. Date: Aug. 16, 2018

(54) Reagents and Methods for miRNA Expression Analysis and Identification of Cancer Biomarkers

(71) Applicant: Wisconsin Alumni Research Foundation, Madison, WI (US)

(72) Inventors: Paul Gerald Ahlquist, Madison, WI (US); Srikumar Sengupta, Madison, WI (US); Johan Arie den Boon, Madison, WI (US); Bill Sugden, Madison, WI (US); Michael Abbott Newton, Madison, WI (US)

(21) Appl. No.: 15/880,007

(22) Filed: Jan. 25, 2018

Related U.S. Application Data
(63) Continuation of application No. 15/013,189, filed on Feb. 2, 2016, now abandoned.
Application No. 15/013,189 is a continuation of application No. 12/116,815, filed on May 7, 2008, now abandoned.
(60) Provisional application No. 60/942,601, filed on Jun. 7, 2007.

(51) Int. Cl. C12Q 1/6886; C12Q 1/6809; C12N 15/113

(52) U.S. Cl. CPC C12Q 1/6886; C12N 2320/11; C12Q 2600/106; C12Q 2600/158; C12Q 1/6809; C12Q 2525/207; C12N 2310/14; C12Q 2600/178; C12N 2330/10; C12Q 2537/143; C12N 15/113

(57) ABSTRACT

This invention provides methods for amplifying, detecting, measuring, and identifying miRNAs from biological samples, particularly limited amounts of a biological sample. miRNAs that are differentially expressed in tumor samples and normal tissues are useful as cancer biomarkers for cancer diagnostics.

This application is a continuation of U.S. Ser. No. 12/116,815, filed May 7, 2008, which claims priority to U.S. provisional application, Ser. No. 60/942,601, filed Jun. 7, 2007. Both applications are incorporated by reference herein in their entirety.

This invention was made with government support under CA097944 and CA022443 awarded by the National Institutes of Health. The government has certain rights in the invention.

### FIELD OF THE INVENTION

The invention provides methods and reagents for amplifying and detecting microRNAs (miRNAs). More particularly, the invention provides methods and reagents for amplifying, measuring, and identifying miRNAs from limited tissue samples or cell samples. In addition, the invention provides bioinformatical methods for miRNA target identification by analyzing correlations between expression of miRNAs and their candidate target mRNAs. Such methods are useful for discovering miRNA cancer biomarkers and for cancer diagnostics.

### BACKGROUND OF THE INVENTION

miRNAs are short (˜22 nucleotides) non-coding RNAs involved in post-transcriptional silencing of target genes. In animals, miRNAs control target gene expression both by inhibiting translation and by marking their target mRNAs for degradation. Although much less common, recent reports indicate that miRNAs can also stimulate target gene expression (Buchan et al., 2007, Science 318: 1877-8; Vasudevan et al., 2007, Science 318: 1931-34; Vasudevan et al., 2007, Cell: 128:1105-118; Bhattacharyya et al., 2007, Cell: 128: 1105-118; Wu et al., 2008, Mol Cell 29: 1-7). The mechanism of miRNA action is through binding to the 3′ untranslated regions (UTRs) of target mRNAs, with varying degrees of sequence complementarity (Bartel, 2004, Cell 116: 281). miRNAs regulate genes associated with development, differentiation, proliferation, apoptosis and stress response, but have also been implicated in multiple cancers, for example: miR-15 and miR-16 in B-cell chronic lymphocytic leukemias (Calin et al., 2002, Proc Natl Acad Sci USA. 99:15524-9; Calin et al., 2004, Proc Natl Acad Sci USA. 101:11755-60); miR-143 and miR-145 in colorectal cancer (Michael et al., 2003, Mol Cancer Res. 1:882-91); miR-125b, miR-145, miR-21, miR-155 and miR-17-5p in breast cancer (Iorio et al., 2005, Cancer Res. 65:7065-70; Hossain et al., 2006, Mol Cell Biol. 26:8191-201); and miR-21 in glioblastoma (Chan et al., 2005, Cancer Res. 65:6029-33). Several miRNAs have been mapped to cancer-associated genomic regions (Calin et al., 2004, Proc Natl Acad Sci USA. 101:2999-3004). The expression of the let-7 miRNA has been correlated with prognosis in lung cancer (Takamizawa et al., 2004, Cancer Res. 64:3753-6) and found to regulate RAS in the same tumor (Johnson et al., 2005, Cell. 120:635-47). Very recently, mir-10b has been shown to contribute to metastasis in breast cancer (Ma et al., 2007, Nature. 449:682-88). This evidence indicates that miRNAs likely affect the development and maintenance of a variety of cancers. Although many miRNAs have been implicated in regulating cancers, very few of their target genes, and hence their downstream mode of action, have been identified.

Tumors often are heterogeneous in cell content, with the true tumor cell mass interspersed with or in close proximity to non-tumor cells. To determine miRNA levels that reflect the status of the tumor cells, measurements derived from stromal and other contaminating cells present in the tumor need to be excluded. This can be achieved by isolating the tumor cells using, inter alia, laser capture-microdissection (LCM) from thin sections of the tumor mass. Although this process achieves isolation of a pure population of the desired cell type(s), the number of cells obtained is limited, and consequently, yields of RNA are low. There is a need in the art, accordingly, for methods permitting miRNA expression detection and profiling from very limited amounts of starting RNA such as obtained from cells isolated by LCM.

The association of miRNA molecules with certain cancers illustrates the need for using the expression levels of these molecules as biomarkers for cancer diagnostics. There is an equally important need to identify mRNA targets of said miRNAs, in order to identify the affected cellular genes and processes involved in tumor initiation, progression and metastasis.

### SUMMARY OF INVENTION

The invention provides methods for amplification and measurement of levels of a plurality of miRNAs in a biological sample, preferably comprising all or a substantial portion thereof of miRNAs in a sample. In addition, the invention provides methods for assessing miRNA profile complexity, preferably in limited amounts of a biological cell or tissue samples and most particularly, in limited amounts of tumor samples. The disclosed methods include assessment of miRNA levels and related mRNA levels, to identify miRNA-specific target mRNAs. One application of said methods is thus to identify cancer biomarkers among both miRNA and target genes.

In the practice of the methods of this invention, oligonucleotide primers are ligated exclusively to miRNAs in RNA extracts from a cell or tissue sample, followed by a series of amplification steps to generate multiple miRNA copies (a non-limiting, exemplary illustration of said methods is shown in FIG. 1. During amplification, miRNA copies are extended with a capture sequence to facilitate detection. The miRNA copies, which have miRNA polarity, are in certain embodiments subsequently hybridized to complementary probes affixed to a microarray, and quantitatively visualized by secondary hybridization of a fluorophore probe that hybridizes specifically to the capture sequence. Alternatively, complementary probes may be fixed to other surfaces such as beads or columns. Detection by secondary hybridization may be performed by a variety of means known in the art, including antibody, enzymatic and colorimetric assays.

In certain embodiments, the invention provides methods for measuring differential expression of miRNAs between control samples and experimental samples. miRNA levels in experimental samples, such as diseased or cancerous tissue sections, are measured and compared to miRNA levels present in control or non-diseased tissues, most preferably wherein the control or non-diseased tissue is from the same tissue source (e.g., normal colon epithelia vs. colon cancer). miRNA species whose levels have the greatest difference between experimental and control tissues are designated as biomarker candidates.

Because miRNAs function by regulating gene expression post-transcriptionally, identification of the target mRNAs complementary to miRNA biomarkers assists in the elucidation of the molecular basis of malignancy and/or disease pathology. This aspect of the invention also identifies additional cancer biomarkers, and particularly biomarkers that can be detected using additional methodologies, including inter alia antibody detection of mRNA gene product(s). Thus, the invention provides methods for identifying downstream mRNA targets of miRNA inactivation that are associated with a cancer phenotype. Candidate miRNA target mRNAs are defined by having sequence complementarity, particularly in their 3′ untranslated region (3′-UTR), to a particular miRNA (as illustrated in FIG. 2). To confirm the identity of said miRNA-complementary mRNA targets among these candidates, the invention is used to measure miRNA levels, and the mRNA levels in the same experimental and control tissues are measured using established methods. Candidate mRNA targets whose differential expression is inversely correlated with the differential expression of their cognate miRNAs, are identified as confirmed targets. Moreover, the methods provided herein are not limited to cancer or the cancer phenotype, but can be used for any disease state showing differential gene expression mediated by miRNA silencing of disease-associated genes.

In addition to these methods, the invention provides a particular miRNA species, miR-29c, as a cancer biomarker for nasopharyngeal carcinoma. The invention provides a plurality of downstream mRNA targets of miR-29c, including several genes expressing extracellular matrix proteins (ECMs). The measurement of miR-29c and/or its target mRNAs in patient samples thus comprises a cancer diagnostic reagent. As demonstrated, by the experimental evidence disclosed herein, miR-29c downregulates expression of multiple genes encoding ECM components or genes related to ECM when an miR-29c-encoding construct is artificially transfected into cells in culture. The ECM related genes whose expression is downregulated by miR-29c include Collagens 1A2 (GenBank Accession No. NM_000089), 3A1 (NM_000090), 4A1 (NM_001845), 15A1 (NM_001855), Laminin-γl (NM_002293) and Fibrillinl. miR-29c also downregulates Thymine-DNA glycosylase (TDG) (NM_003211) and FUSIP1 (NM_006625, NM_054016) (shown in FIG. 3; Table 5). Reference Sequence Identifiers are shown in parentheses.

Advantages of the practice of this invention include, inter alia, that it permits measurement of miRNA expression levels in enriched tumor cell populations from patient biopsies isolated by methods such as LCM, from limited tumor cell sources that, prior to this invention, yielded insufficient total RNA for miRNA expression profiling.

Specific preferred embodiments of the present invention will become evident from the following more detailed description of certain preferred embodiments and the claims.

### BRIEF DESCRIPTION OF THE DRAWINGS

These and other objects and features of this invention will be better understood from the following detailed description taken in conjunction with the drawing wherein:

FIG. 1 is an outline of a method used to measure miRNA expression from microdissected cells isolated from patient biopsies, illustrating amplification and a two-step hybridization process. One embodiment of the method set forth in this Figure was practiced as described in detail in Example 5.

FIG. 2A and FIG. 2B show miR-29c target sites in predicted target mRNAs. Potential binding sites for miR-29c in the target mRNAs, including the 5′ miRNA seed sequence (underlined), are shadowed. The sequences disclosed in the figure include miR-29c 5′ UAGCACCAUUUGAAAUCGGU 3′ (SEQ ID NO: 1). The same miR-29c sequence is also represented throughout the FIG. 2 in a 3′ to 5′ direction.

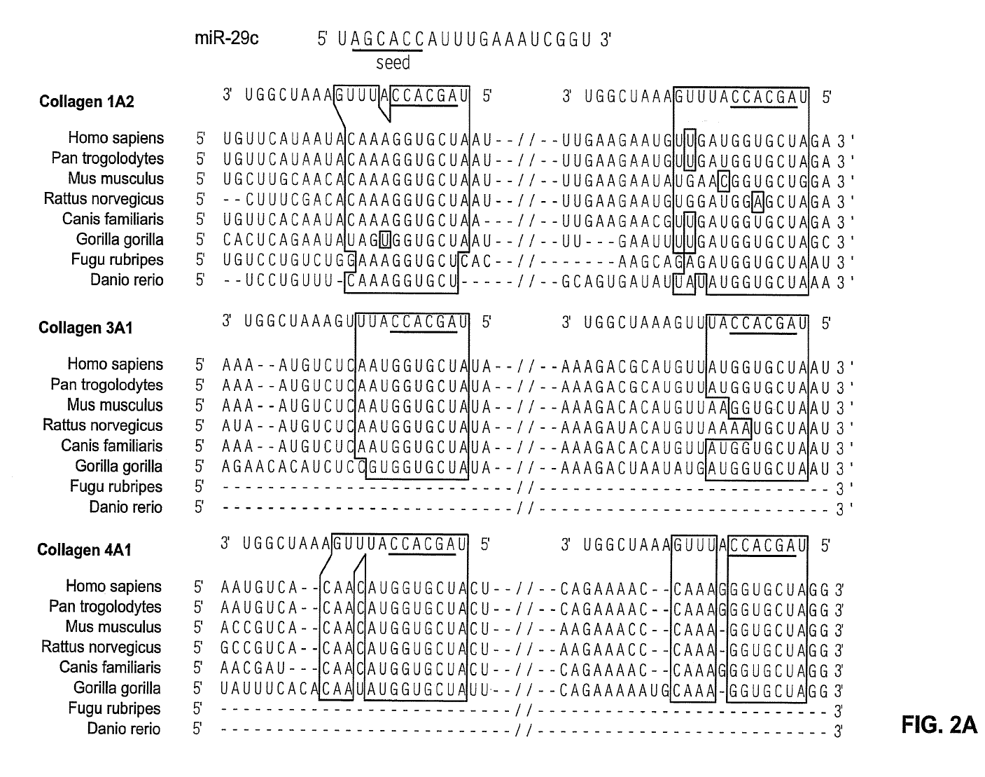

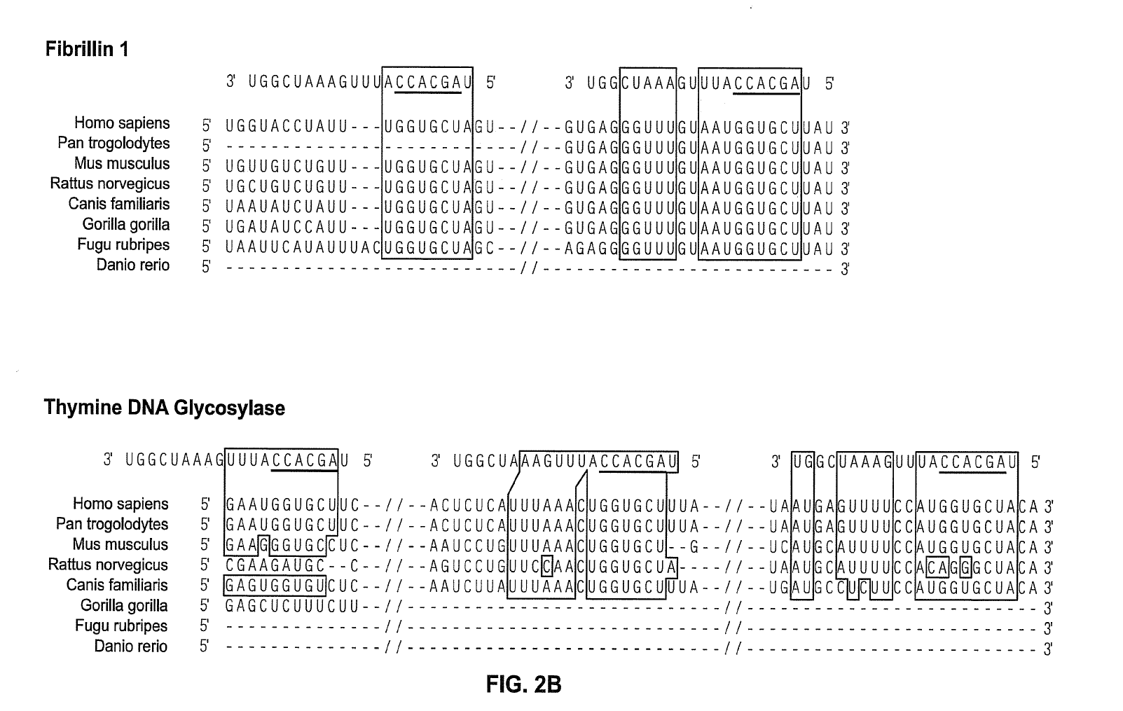

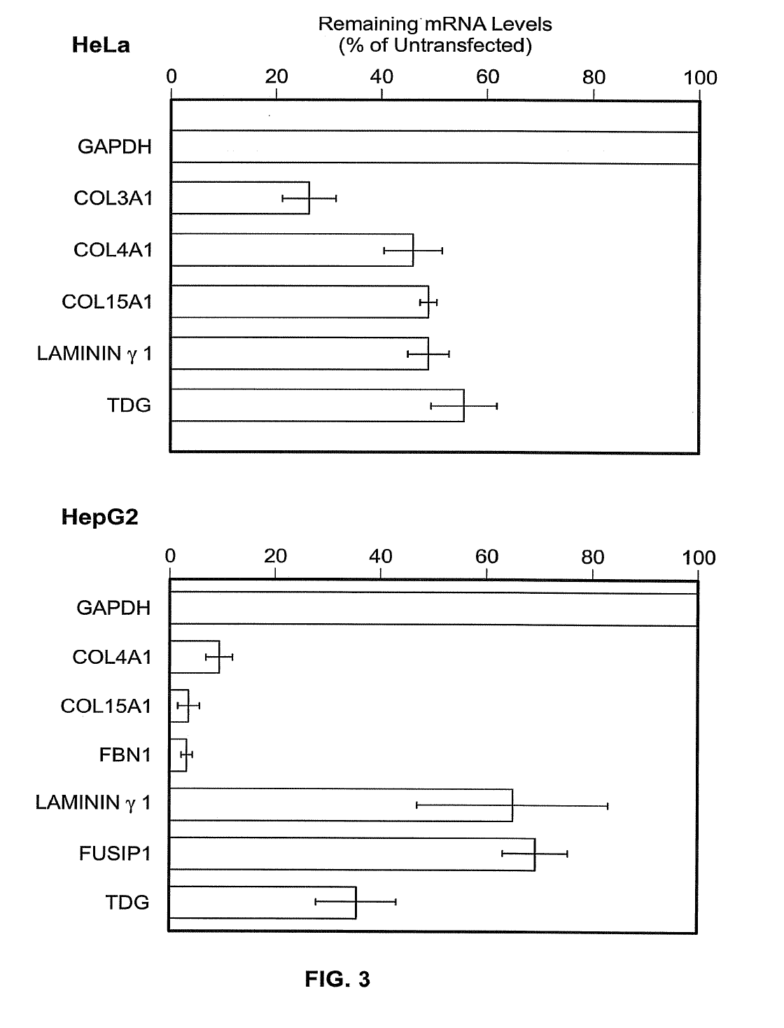

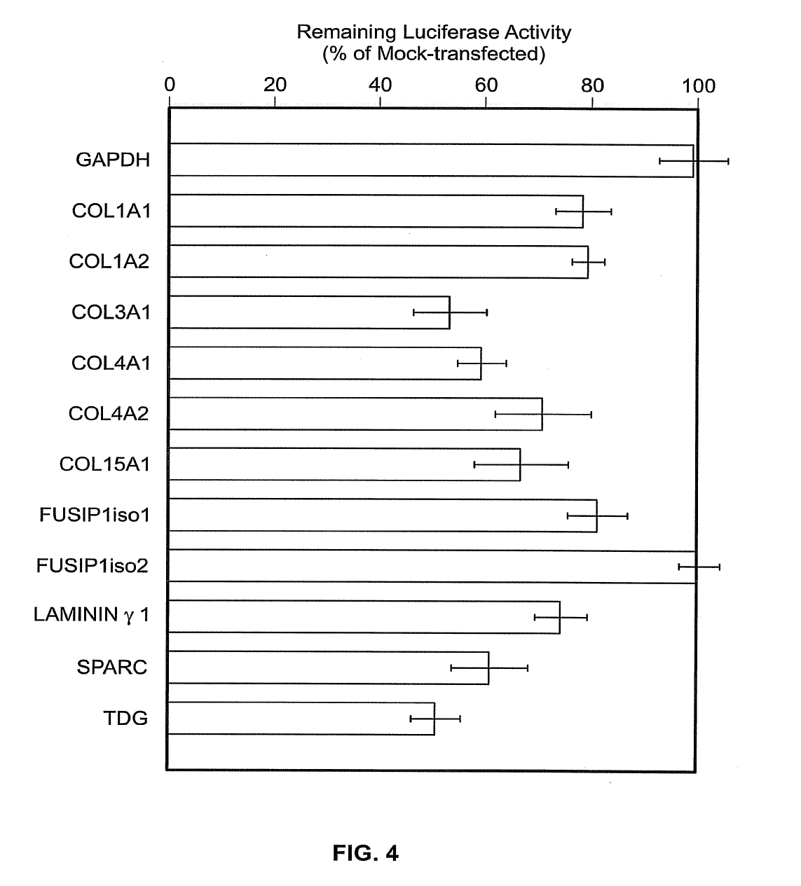

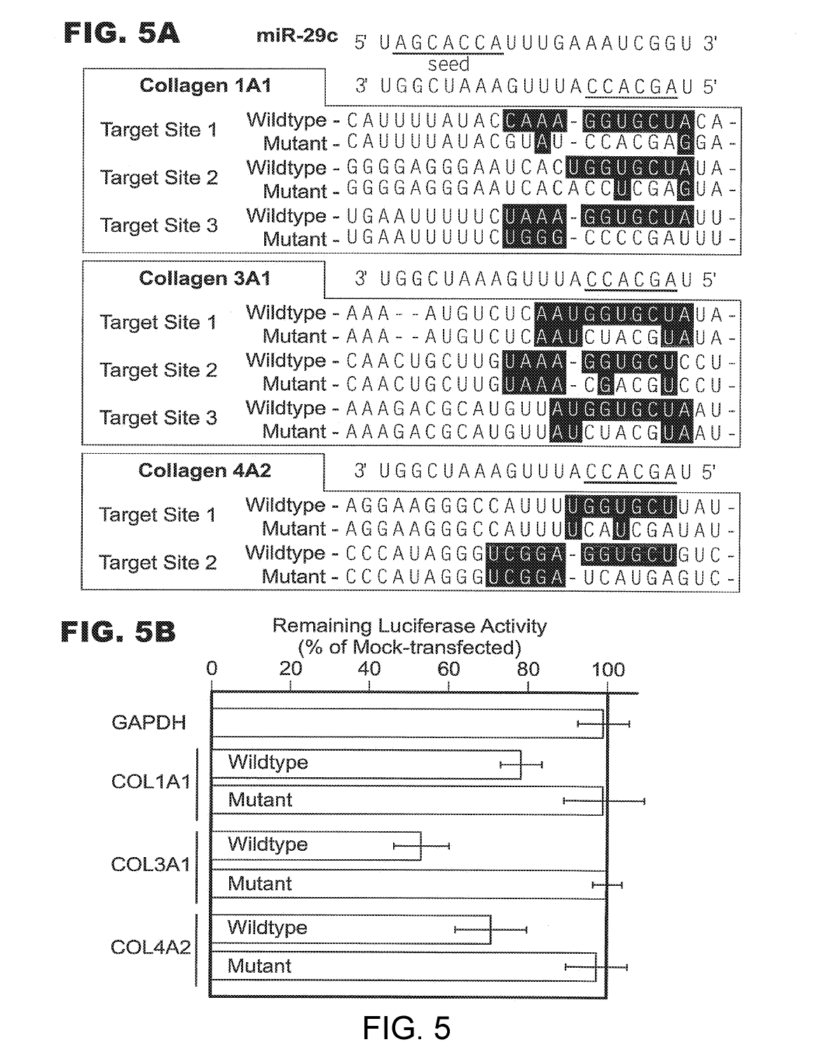

The sequence identifiers for the sequences disclosed in FIG. 2 are provided in the following paragraphs. Collagen 1A2 homo sapiens upstream sequence (SEQ ID NO: 2) and downstream sequence (SEQ ID NO: 3); Collagen 1A2 Pan trogolodytes upstream sequence (SEQ ID NO: 4) and downstream sequence (SEQ ID NO: 5); Collagen 1A2 Mus musculus upstream sequence (SEQ ID NO: 6) and downstream sequence (SEQ ID NO: 7); Collagen 1A2 Rattus norvegicus upstream sequence (SEQ ID NO: 8) and downstream sequence (SEQ ID NO: 9); Collagen 1A2 Canis familiaris upstream sequence (SEQ ID NO: 10) and downstream sequence (SEQ ID NO: 11); Collagen 1A2 Gorilla gorilla upstream sequence (SEQ ID NO: 12) and downstream sequence (SEQ ID NO: 13); Collagen 1A2 Fugu rubripes upstream sequence (SEQ ID NO: 14) and downstream sequence (SEQ ID NO: 15); Collage 1A2 Danio rerio upstream sequence (SEQ ID NO: 16) and downstream sequence (SEQ ID NO: 17).

Collagen 3A1 homo sapiens upstream sequence (SEQ ID NO: 18) and downstream sequence (SEQ ID NO: 19); Collagen 3A1 Pan trogolodytes upstream sequence (SEQ ID NO: 20) and downstream sequence (SEQ ID NO: 21); Collagen 3A1 Mus musculus upstream sequence (SEQ ID NO: 22) and downstream sequence (SEQ ID NO: 23); Collagen 3A1 Rattus norvegicus upstream sequence (SEQ ID NO: 24) and downstream sequence (SEQ ID NO: 25); Collagen 3A1 Canis familiaris upstream sequence (SEQ ID NO: 26) and downstream sequence (SEQ ID NO: 27); Collagen 3A1 Gorilla gorilla upstream sequence (SEQ ID NO: 28) and downstream sequence (SEQ ID NO: 29).

Collagen 4A1 homo sapiens upstream sequence (SEQ ID NO: 30) and downstream sequence (SEQ ID NO: 31); Collagen 4A1 Pan trogolodytes upstream sequence (SEQ ID NO: 32) and downstream sequence (SEQ ID NO: 33); Collagen 4A1 Mus musculus upstream sequence (SEQ ID NO: 34) and downstream sequence (SEQ ID NO: 35); Collagen 4A1 Rattus norvegicus upstream sequence (SEQ ID NO: 36) and downstream sequence (SEQ ID NO: 37); Collagen 4A1 Canis familiaris upstream sequence (SEQ ID NO: 38) and downstream sequence (SEQ ID NO: 39); Collagen 4A1 Gorilla gorilla upstream sequence (SEQ ID NO: 40) and downstream sequence (SEQ ID NO: 41).

Fibrillin 1 homo sapiens upstream sequence (SEQ ID NO: 42) and downstream sequence (SEQ ID NO: 43); Fibrillin 1 Pan trogolodytes downstream sequence (SEQ ID NO: 44); Fibrillin 1 Mus musculus upstream sequence (SEQ ID NO: 45) and downstream sequence (SEQ ID NO: 46); Fibrillin 1 Rattus norvegicus upstream sequence (SEQ ID NO: 47) and downstream sequence (SEQ ID NO: 48); Fibrillin 1 Canis familiaris upstream sequence (SEQ ID NO: 49) and downstream sequence (SEQ ID NO: 50); Fibrillin 1 Gorilla gorilla upstream sequence (SEQ ID NO: 51) and downstream sequence (SEQ ID NO: 52); Fibrillin 1 Fugu rubripes upstream sequence (SEQ ID NO: 53) and downstream sequence (SEQ ID NO: 54).

Thymine DNA Glycosylase homo sapiens upstream sequence (SEQ ID NO: 55), middle sequence (SEQ ID NO: 56) and downstream sequence (SEQ ID NO: 57); Thymine DNA Glycosylase Pan trogolodytes upstream sequence (SEQ ID NO: 58), middle sequence (SEQ ID NO: 59) and downstream sequence (SEQ ID NO: 60); Thymine DNA Glycosylase Mus musculus upstream sequence (SEQ ID NO: 61), middle sequence (SEQ ID NO: 62) and downstream sequence (SEQ ID NO: 63); Thymine DNA Glycosylase Rattus norvegicus upstream sequence (SEQ ID NO: 64), middle sequence (SEQ ID NO: 65) and downstream sequence (SEQ ID NO: 66); Thymine DNA Glycosylase Canis familiaris upstream sequence (SEQ ID NO: 67), middle sequence (SEQ ID NO: 68) and downstream sequence (SEQ ID NO: 69); Thymine DNA Glycosylase Gorilla gorilla upstream sequence (SEQ ID NO: 70).

FIG. 3 illustrates miR-29c-mediated downregulation of target mRNA accumulation. HeLa and HepG2 cells transfected with miR-29c precursor have lower levels of the target mRNAs than untransfected cells as measured by quantitative real time PCR using equal amounts of total cellular RNA. mRNA levels were normalized to those in the untransfected cells.

FIG. 4 illustrates miR-29c-mediated inhibition of miR-29c target genes. 3′ UTRs of target genes containing mir-29c binding sites were cloned into vectors containing firefly luciferase that were transfected into HeLa cells. These cells were subsequently transfected with mir-29c precursor RNAs or mock-transfected. Compared to cells that were mock-transfected (where the detected luciferase activity was considered 100%), mir-29c precursor-transfected cells showed a reduction in luciferase activity.

FIG. 5 illustrates the effects of mutations that disrupt mir-29c binding to 3′ UTRs of three target genes, wherein mir-29c binding-site mutations prevented mir-29c-mediated inhibition of gene target gene expression. FIG. 5A shows nucleotides (black box) in the mRNA sequence indicating the extent of basepairing with mir-29c, and in particular how the mutations disrupt basepairing with the mir-29c seed sequence.

The sequences disclosed in the figure include miR-29c 5′ UAGCACCAUUUGAAAUCGGU 3′ (SEQ ID NO: 1). The same miR-29c sequence is also represented throughout the FIG. 5A in a 3′ to 5′ direction. Collagen 1A1: Target Site 1: Wildtype (SEQ ID NO: 564) and Mutant (SEQ ID NO: 565); Target Site 2: Wildtype (SEQ ID NO: 566) and Mutant (SEQ ID NO: 567); Target Site 3: Wildtype (SEQ ID NO: 568) and Mutant (SEQ ID NO: 569). Collagen 3A1: Target Site 1: Wildtype (SEQ ID NO: 570) and Mutant (SEQ ID NO: 571); Target Site 2: Wildtype (SEQ ID NO: 572) and Mutant (SEQ ID NO: 573); Target Site 3: Wildtype (SEQ ID NO: 574) and Mutant (SEQ ID NO: 575). Collagen 4A2: Target Site 1: Wildtype (SEQ ID NO: 576) and Mutant (SEQ ID NO: 577); Target Site 2: Wildtype (SEQ ID NO: 578) and Mutant (SEQ ID NO: 579).

FIG. 5B shows the results of luciferase activity assays in HeLa cells comprising wildtype or mutated 3′ UTRs of target mRNAs cloned into vectors containing firefly luciferase for expression, transfected with precursor mir-29c RNA or mock-transfected. Luciferase activity was not affected by mir-29c expression in cells transfected with constructs containing the mutated target sequence.

### DETAILED DESCRIPTION OF PREFERRED EMBODIMENTS

This invention provides methods and reagents for measuring miRNA expression in a biological sample, preferably a cell or tissue sample and even more preferably a tumor sample, and particularly when the amounts of such samples are limited in size and/or the number of cells. The term “limited” as used herein refers preferably to a range of approximately 1000-10,000 cells. In a preferred embodiment, cell numbers range from approximately 1000-10,000 cells, or alternatively 1000-5000 cells, in certain alternative embodiments approximately 1000 cells or in certain samples from about 500-1000 cells, in yet other samples 10-500 cells or at a minimum at least one cell.

In turn, the methods disclosed herein permit miRNA expression from minute amounts of starting RNA to be identified. The term “minute” as used herein refers to very low amounts of total RNA. In a preferred embodiment, starting RNA will comprise about 30-100 ng of RNA, preferably 50-90 ng, and more preferably 75-85 ng. The invention thus provides methods for assessing differential expression of miRNA species between biological samples, particularly cell or tissue samples and even more preferably tumor samples, and control, preferably non-tumor samples, wherein the tumor samples are enriched for tumor cell content as described herein. The invention also provides methods for identifying one or a plurality of miRNA-complementary target mRNAs from cellular genes whose expression is modulated (upregulated or downregulated) by expression of one or a plurality of miRNA species. The inventive methods are useful for the identification of disease biomarkers, particularly cancer biomarkers.

The term “biomarker” as used herein refers to miRNA, mRNA or protein species that exhibit differential expression between biological samples, preferably patient samples and more preferably cancer patient samples, when compared with control patient samples. The term “patient sample” as used herein refers to a cell or tissue sample obtained from a patient (such as a biopsy) or cells collected from in vitro cultured samples; the term can also encompass experimentally derived cell samples. In a preferred embodiment, patient samples are laser-microdissected, inter alia from frozen tissue sections. Cells from patient samples can be used directly after isolation from biopsy material or can be in vitro propagated.

As used herein, the terms “experimental sample” and “biological sample” refer preferably to a diseased or cancerous tissue sample including specifically cell culture samples and experimentally-derived samples. As used herein, the term “control” sample refers to tissue that is normal or pathology-free in appearance and may be harvested from the same patient or a different patient, most preferably being from the same tissue type as the disease or experimental sample (e.g., normal colon tissue vs. colon cancer) and most preferably otherwise processed as is an experimental, biological or patient sample. The term “tumor” refers to a tissue sample or cells that exhibit a cancerous morphology, express cancer markers, or appear abnormal, or that have been removed from a patient having a clinical diagnosis of cancer. A tumorigenic tissue is not limited to any specific stage of cancer or cancer type, an expressly includes dysplasia, anaplasia and precancerous lesions such as inter alia adenoma. As used herein, the term “disease” or “diseased” refers to any abnormal pathologies, including but not limited to cancer. As used herein, the term “aberrant” refers to abnormal or altered.

As designated herein, miRNA targets are mRNA transcripts that are regulated by miRNA. Regulation of target mRNA can include but is not limited to binding or any sequence-specific interaction between an miRNA and its target mRNA, and includes but it not limited to decreasing stability of the mRNA, or decreasing mRNA translation, or increasing mRNA degradation.

The practice of this invention can involve procedures well-known in the art, including for example nucleotide sequence amplification, such as polymerase chain reaction (PCR) and modifications thereof (including for example reverse transcription (RT)-PCR, and stem-loop PCR), as well as reverse transcription and in vitro transcription. Generally these methods utilize one or a pair of oligonucleotide primers having sequence complimentary to sequences 5′ and 3′ to the sequence of interest, and in the use of these primers they are hybridized to a nucleotide sequence and extended during the practice of PCR amplification using DNA polymerase (preferably using a thermal-stable polymerase such as Taq polymerase). RT-PCR may be performed on miRNA or mRNA with a specific 5′ primer or random primers and appropriate reverse transcription enzymes such as avian (AMV-RT) or murine (MMLV-RT) reverse transcriptase enzymes.

The term “complimentary” as used herein refers to nucleotide sequences in which the bases of a first oligonucleotide or polynucleotide chain are able to form base pairs with a sequence of bases on another oligonucleotide or polynucleotide chain. The terms “sense” and “antisense” refer to complimentary strands of a nucleotide sequence, where the sense strand or coding strand has the same polarity as an mRNA transcript and the antisense strand or anticoding strand is the coding strand's compliment. The antisense strand is also referred to as the anticoding strand. The term “hybridization” as used herein refers to binding or interaction of complementary nucleotide strands, particularly wherein the complementary bases in the two chains form intermolecular hydrogen bonds between the bases (known in the art as “basepairing”). Hybridization need not be 100% complete base pair matching, meaning some of the bases in a given set of sequences need not be complimentary, provided that enough of the bases are complimentary to permit interaction or annealing of the two strands under the conditions specified, including temperature and salt concentration. In certain embodiments of the invention, hybridization occurs between miRNAs and their target mRNAs, which is often imperfect (e.g. less than 100% complimentary base pairing). miRNAs inhibit translation of target mRNAs by binding to target sequences with which they share at least partial complementarity, wherein said target sequences are most often located within the 3′ untranslated region (UTR) of these target mRNAs. It will be recognized that this is not always a simple function of calculating purported or proposed specificities, since secondary structures (stem-and-loop structures, for example) can affect the stability or accessibility of miRNA/mRNA hybridization. Accordingly, hybridization is most accurately measured by detecting decreased expression of a target mRNA in a cell expressing the complementary miRNA; these methods for detecting intracellular hybridization are also specific for functional miRNA::mRNA hybridization events. Conversely, hybridization between a capture sequence and its corresponding probe will typically have near-perfect to perfect (complete) base pairing (i.e. the sequence experiences extensive complimentary base pairing for a particular sequence or portion of a transcript).

The term “sense targets” as used herein refers to sense strands of miRNA containing a capture sequence. The sense targets are generated by the methods of the invention as disclosed herein. Sense targets can be detected and identified using antisense (i.e., complementary) RNA. In a preferred embodiment, antisense miRNAs are bound to a microarray that is used to detect such sense targets.

The term “capture sequence” as used herein refers to any nucleotide sequence used to hybridize with a detection probe. In a preferred embodiment, the capture sequence is SEQ ID NO: 71. TTC TCG TGT TCC GTT TGT ACT CTA AGG TGG A. This sequence is used in the methods of the invention to identify miRNAs amplified from a sample, which were bound to probe miRNAs affixed to a microarray. In a second hybridization step, a fluorophore-labeled detection probe, with oligonucleotide sequence complementary to the capture sequence, was used to detect those sample miRNAs that bound to the microarray.

The terms “secondary detection probe” or “secondary hybridization” refer to the use of a second hybridization step in a microarray or other hybridization-based analysis. In a preferred embodiment, the capture sequence in amplified miRNAs bound to the microarray by a primary hybridization step is used to hybridize to a complementary oligonucleotide that is linked to a fluorophore, most preferably using fluorescent labels that have excitation and emission wavelengths adapted for detection using commercially-available instruments. Examples of fluorescent labels useful in the practice of the invention include CY3 3DNA™ (Genisphere, Pa., USA), phycoerythrin (PE), fluorescein isothiocyanate (FITC), rhodamine (RH), Texas Red (TX), Cy3, Hoechst 33258, and 4′,6-diamidino-2-phenylindole (DAPI). The fluorophore complex in particular permits detection of miRNA by automated microarray scanners.

The term “inversely proportional” as used herein refers to the comparison of expression levels of miRNAs and mRNAs between tissue samples or groups of similar samples. For example, where miRNA expression levels are low in a cancer sample, the methods of the invention identify high miRNA expression in control samples. This differential expression analysis permits identification of potential cancer markers. In a preferred embodiment, the invention identifies mRNAs that are expressed at levels inversely proportional to regulatory miRNAs. For example, where miRNAs are expressed at high levels in a cancer tissue, the methods identify mRNAs that are expressed at low levels in the cancer tissue, since the miRNAs affect mRNA expression in the cancer cell.

The terms “differential analysis” and “differentially expressed” as used herein may refer to, but are not limited to differences in expression levels for miRNAs and/or mRNAs between control and experimental samples. Alternatively, as described above, differential analysis may also include comparisons of expression between miRNAs and potential target mRNAs within the same tissue sample or different tissue samples. In addition, the terms as used herein may refer to the expression of miRNA at greater or lesser amounts in an experimental tissue/experimental cell sample than miRNA expression in a control cell/control tissue sample. The control sample can be from healthy tissue from the same patient or a different patient. Expression of miRNAs may occur in a tissues sample where typically expression does not occur, or expression may occur at levels greater than or less than typically found in a particular cell or tissue type. An example of such differential expression is demonstrated herein for miR-29c in nasopharyngeal carcinoma, as discussed more fully below.

The term “miRNA specific primers” as used herein refers to 3′ and 5′ primers that link to miRNA and permit miRNA amplification. Primers for amplifying miRNA are commercially available and techniques are known in the art. (see, for example, Lau et al., 2001, Science. 294:858-62). In use, primers are ligated to all single-stranded RNA species with a free 3′0H and a 5′ monophosphate, which includes all miRNAs (and specifically excludes eukaryotic mRNA).

As used herein, the terms “microarray,” “bioarray,” “biochip” and “biochip array” refer to an ordered spatial arrangement of immobilized biomolecular probes arrayed on a solid supporting substrate. Preferably, the biomolecular probes are immobilized on the solid supporting substrate.

Gene arrays or microarrays as known in the art are useful in the practice of the methods of this invention. See, for example, DNA MICROARRAYS: A PRACTICAL APPROACH, Schena, ed., Oxford University Press: Oxford, UK, 1999. As used in the methods of the invention, gene arrays or microarrays comprise a solid substrate, preferably within a square of less than about 22 mm by 22 mm on which a plurality of positionally-distinguishable polynucleotides are attached at a diameter of about 100-200 microns. These probe sets can be arrayed onto areas of up to 1 to 2 cm², providing for a potential probe count of >30,000 per chip. The solid substrate of the gene arrays can be made out of silicon, glass, plastic or any suitable material. The form of the solid substrate may also vary and may be in the form of beads, fibers or planar surfaces. The sequences of the polynucleotides comprising the array are preferably specific for human miRNA. The polynucleotides are attached to the solid substrate using methods known in the art (Schena, Id.) at a density at which hybridization of particular polynucleotides in the array can be positionally distinguished. Preferably, the density of polynucleotides on the substrate is at least 100 different polynucleotides per cm², more preferably at least 300 polynucleotides per cm². In addition, each of the attached polynucleotides comprises at least about 25 to about 50 nucleotides and has a predetermined nucleotide sequence. Target RNA or cDNA preparations are used from tumor samples that are complementary to at least one of the polynucleotide sequences on the array and specifically bind to at least one known position on the solid substrate.

Gene arrays are complex experimental systems, and their development stemmed from a confluence of various technologies including the Human Genome Project and the development of computational power and bioinformatics applications to DNA sequencing, probe design, and data output analysis (Lockhart et al., 2000, Nature 405: 827-36; Schena et al., 1998, Trends Biotechnol. 16: 301-6; Schadt et al., 2000, J. Cell Biochem. 80: 192-202; Li et al., 2001, Bioinformatics 17: 1067-1076; Wu et al., 2001, Appl. Environ. Microbiol. 67: 5780-90; and Kaderali et al., 2002, Bioinformatics 18: 1340-9). These developments enable one of ordinary skill to produce arrays of polynucleotides from a plurality of different human genes, including polynucleotides complementary to miRNA species.

Two principal array platforms are currently in widespread use, but differ in how the oligonucleotide probes are placed onto the hybridization surface (Lockhart et al., 2000, Id. and Gerhold et al., 1999, Trends Biochem. Sci. 24: 168-73). Schena and Brown pioneered techniques for robotically depositing presynthesized oligonucleotides (typically, PCR-amplified inserts from cDNA clones) onto coated surfaces (Schena et al., 1995, Science 270: 467-70 and Okamoto et al., 2000, Nat. Biotechnol. 18: 438-41). Fodor et al. (1991, Science 251: 767-73) and Lipshutz et al. (1999, Nat. Genet. 21:20-4), on the other hand, utilized photolithographic masking techniques (similar to those used to manufacture silicon chips) to construct polynucleotides one base at a time on preferentially unmasked surfaces containing an oligonucleotide targeted for chain elongation. These two methods generate reproducible probe sets amenable for gene expression profiling and can be used to determine the gene expression profiles of tumor samples when used in accordance with the methods of this invention.

Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample. As provided herein, the invention comprises useful microarrays for detecting differential miRNA expression in tumor samples, prepared as set forth herein or provided by commercial sources, such as Affymetrix, Inc. (Santa Clara, Calif.), Incyte Inc. (Palo Alto, Calif.) and Research Genetics (Huntsville, Ala.).

In certain embodiments of the diagnostic methods of this invention, said biochip arrays are used to detect differential expression of miRNA or target mRNA species by hybridizing amplification products from experimental and control tissue samples to said array, and detecting hybridization at specific positions on the array having known complementary sequences to specific miRNAs or their mRNA target(s).

In certain other embodiments of the diagnostic methods of this invention, expression of the protein product(s) of mRNA targets of miRNA regulation are detected. In preferred embodiments, protein products are detected using immunological reagents, examples of which include antibodies, most preferably monoclonal antibodies that recognize said differentially-expressed proteins.

For the purposes of this invention, the term “immunological reagents” is intended to encompass antisera and antibodies, particularly monoclonal antibodies, as well as fragments thereof (including F(ab), F(ab)₂, F(ab)' and Fᵥ fragments). Also included in the definition of immunological reagent are chimeric antibodies, humanized antibodies, and recombinantly-produced antibodies and fragments thereof. Immunological methods used in conjunction with the reagents of the invention include direct and indirect (for example, sandwich-type) labeling techniques, immunoaffinity columns, immunomagnetic beads, fluorescence activated cell sorting (FACS), enzyme-linked immunosorbent assays (ELISA), and radioimmune assay (MA).

The immunological reagents of the invention are preferably detectably-labeled, most preferably using fluorescent labels that have excitation and emission wavelengths adapted for detection using commercially-available instruments such as and most preferably fluorescence activated cell sorters. Examples of fluorescent labels useful in the practice of the invention include phycoerythrin (PE), fluorescein isothiocyanate (FITC), rhodamine (RH), Texas Red (TX), Cy3, Hoechst 33258, and 4′,6-diamidino-2-phenylindole (DAPI). Such labels can be conjugated to immunological reagents, such as antibodies and most preferably monoclonal antibodies using standard techniques (Maino et al., 1995, Cytometry 20: 127-133).

The methods of this invention detect miRNAs differentially expressed in malignant and normal control tissue. Certain embodiments of the methods of the invention can be used to detect differential miRNA expression in Epstein-Barr virus (EBV)-associated nasopharyngeal carcinoma (NPC). NPC is a highly metastatic tumor even in the early stage of the disease (Cassisi: Tumors of the pharynx. Thawley et al., eds. Comprehensive Management of Head and Neck Tumors, 1987, Vol l.:pp 614-683, W. B. Saunders Co., Philadelphia).

Nasopharyngeal carcinoma (NPC) is associated with Epstein-Barr virus (EBV), is found prominently in people in South East Asia, and is highly invasive (Lo et al., 2004, Cancer Cell. 5:423-428). Differential gene expression in NPC relative to normal nasopharyngeal epithelium was examined. Differential expression underlies the properties of this type of tumor, which illustrate the contribution of EBV genes towards immune evasion of tumor cells in this cancer and further implicate DNA repair and nitrosamine metabolism mechanisms in NPC pathogenesis (Sengupta et al., 2006, Cancer Res. 66:7999-8006; Dodd et al., 2006, Cancer Epidemiol Biomarkers Prey. 15:2216-2225).

The invention provides sensitive procedures for amplifying miRNAs from enriched, tumor cell sources, such as laser-microdissected frozen tissue sections (and advantageously assaying a cell or tissue population highly enriched, more preferably very highly enriched, in tumor cells and not stromal or other undesirable cells) and detecting these miRNAs using, for example, microarrays. “Enriched” as used herein indicates that more than approximately 50%, more preferably more than 60%, more than 70%, even more preferably at least 80% and in certain embodiments at least 85%, 86%, 87%, 88%, 89%, 90%, 91%, 92%, 93%, 94%, 95%, 96%, 97%, 98 or 99% of the cells in a sample are of the cells in a sample are of the targeted cell type. The inventive methods have an advantage, inter alia, over traditional methods that require a larger tissue sample that required excision from a patient or alternatively that required that tumor cells from excised tissues be propagated in cell culture, thus relying on the (incomplete) growth advantage of tumor cells over stromal cells, in order to collect sufficient RNA for the subsequent analysis. The differentially-expressed miRNAs detected using the inventive methods thus provided potential tumor markers for malignancy, tumor progression and metastasis.

These inventive methods were able to isolate and amplify minute amounts of miRNA from limited tissue biopsies. For example, needle biopsies typically measure 1mm diameter by 2 mm length, and experimental samples often comprise one or more ˜20 micron cryosections, which contain very little tissue. These samples generally are not 100% pure tumor cell populations, and thus some samples require laser capture of the tumor component to enrich up to the preferred percentage of epithelial cell type.

In order to identify miRNA cancer biomarkers, two hundred twenty-two (222) human miRNAs were analyzed from thirty-one microdissected NPC samples and ten site-matched normal epithelial tissues. Eight cellular miRNAs were found to be differentially expressed between tumor and normal cells. Two algorithms were used to search for target mRNAs regulated by these miRNAs. {See pictar.bio.nyu.edu/cgi-bin/PicTar_vertebrate.cgi, snf (www.targetscan.org as discussed in Example 4).} One of the miRNA species, miR-29c, was found to be downregulated in NPC and associated with post-transcriptional regulation of multiple extra-cellular matrix protein genes. Increased levels of extracellular matrix proteins, particularly collagens and laminins would be expected to increase the invasiveness and metastasis of many tumor cells. The association between differential expression of miR-29c and extracellular matrix protein expression was confirmed in two epithelial cells in culture, where miR-29c expression was increased artificially, resulting in decreased expression of eight cellular mRNAs, six of which encoded extra-cellular matrix (ECM) proteins. Thus, differential expression of miR-29c miRNA in NPC tissue is consistent with its use as a biomarker, since it had the capacity to contribute to pathogenesis of NPC tumors. These results demonstrated that the methods of this invention were useful for identifying miRNA cancer biomarkers and their downstream mRNA targets.

Once detected, differentially amplified and/or overexpressed miRNAs or mRNAs can be used alone or in combination to assay individual tumor samples and determine a prognosis, particularly a prognosis regarding treatment decisions, most particularly regarding decisions relating to treatment modalities such as chemotherapeutic treatment. Moreover, once differentially-expressed miRNA biomarkers have been identified, potential target mRNAs can be identified by detecting target sequences in said mRNAs, particularly in the 3′ UTR thereof, that are complementary to the capture sequences of the differentially-expressed miRNAs.

Finally, the administration of miRNAs as therapeutics is well known in the art. (See, De Fougerolles, 2008, Human Gene Therapy, 19: 125-32 for a recent review.) Examples 5 and 6 herein illustrate miRNA regulation/modulation of target mRNA expression. Hence miR-29c, miR-29a, miR-29b, miR-34c, miR-34b, miR-212, miR-216 and miR-217, miR-151 or miR-192 and other miRNAs identified by the disclosed methods may be administered as therapeutics for the treatment of cancer, including NPC, and other disorders by methods known in the art.

miRNAs identified according to the methods herein provide targets for therapeutic intervention. miRNAs that are underexpressed, such as miR-29c in tumors such as NPC or in other tumors or other diseases or disorders, can be introduced using conventional nucleic acid formulation and delivery methods. (De Fougerolles, 2008, Human Gene Therapy, 19: 125-3; Akinc et al., 27 April 2008, Nature Biotechnology, advanced online: 1-9). Alternatively, expression of endogenous miR-29c in tumors such as NPC or in other tumors or other diseases or disorders, can be increased, inter alia, using stimulators of miRNA expression. Similarly, expression of miRNAs that are overexpressed can be repressed, using antisense or siRNA methods or by modulating expression using repressors of miRNA expression. The invention also contemplates compounds and pharmaceutical compositions thereof and methods for modulating miRNA expression in a tumor or other tissue to achieve a therapeutic effect.

Embodiments of the methods of this invention comprising the above-mentioned features are intended to fall within the scope of this invention.

### EXAMPLES

The Examples which follow are illustrative of specific embodiments of the invention, and various uses thereof. They set forth for explanatory purposes only, and are not to be taken as limiting the invention.

### Example 1

### miRNA Isolation and Amplification

The methods described in this Example were developed to overcome deficiencies in the art associated with detection and differential expression analysis of miRNAs isolated from limited cell or tissue samples.

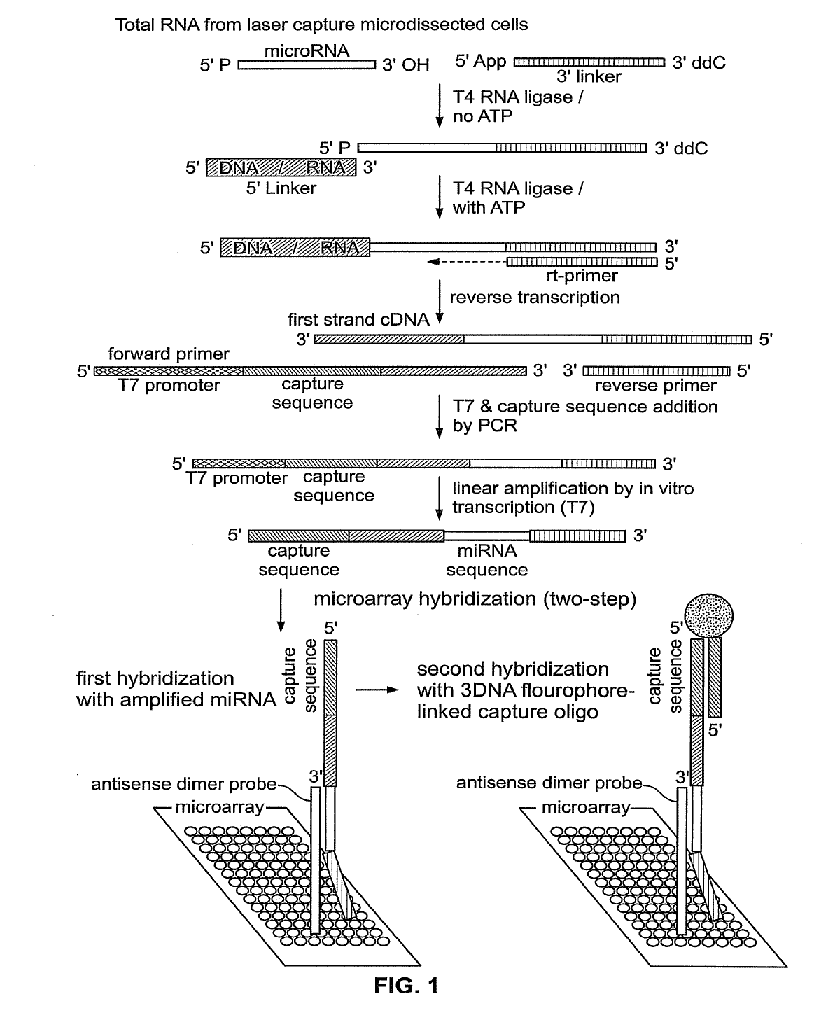

Total cellular RNA was isolated from tissue samples including nasopharyngeal carcinoma (NPC) tissue samples. Collection and processing of such samples, including histopathology, laser capture microdissection, and RNA extraction have been described in detail previously (Sengupta et al., 2006, Cancer Res. 66: 7999-8006), the disclosure of which is incorporated by reference herein. Here, a total of thirty-one NPC samples and ten normal nasopharyngeal tissue samples (including six normal tissue samples from non-NPC or biopsy-negative cases and four samples from tumor free nasopharyngeal area of NPC patients) were used. miRNA was amplified from total RNA isolated from laser microdissected/whole tissue sections without any size selection following the procedures disclosed in Lau et al. (2001, Science. 294:858-62, the disclosure of which is incorporated by reference herein) as briefly set forth as follows and illustrated in FIG. 1.

Total RNA (˜100 ng) from laser microdissected cells (isolated using Trizol, Invitrogen, Carlsbad, Calif., USA) was used in a ligation reaction where all single stranded RNA species with a 3′ OH were ligated using by RNA ligase 1 to a “3′ linker” having the sequence:

(SEQ ID NO: 72)
AppCTG TAG GCA CCA TCA AT(ddC);

this oligonucleotide was commercially-available as a miRNA cloning linker from Integrated DNA Technologies (Coralville, Iowa). The reaction was carried out using a modification of the conventional, two-step reaction (where in the first step, ATP was used to adenylate the 5′ end of a nucleic acid and in the second step, the activated adenylated nucleic acid was ligated to the 3′ OH of another nucleic acid). Here, the presence of a 5′ pyrophosphate on the linker moiety permitted the reaction mixture to exclude ATP, with the consequence that the only RNA species in the reaction mixture capable of being ligated to a 3′OH was the linker; this prevented the ligase from nonspecifically ligating unrelated RNA molecules from the tissue sample in the reaction mixture to one another, as well as preventing individual RNA molecules from being circularized. Finally, the presence of the 3′dideoxy-C (ddC) residue in the linker moiety prevented RNA molecules that were ligated to the linker from further participation in the ligation reaction.
The next step for preparing the RNA population for amplification was ligating a linker to the 5′ end of the RNA molecules in the reaction mixture. For this reaction, a “5′ linker” having the sequence:

(SEQ ID NO: 73)
ATC GTa ggc acc uga aa

(wherein uppercase letters designate deoxyribonucleotide residues and lowercase letters are ribonucleic acid residues; commercially-available from Dharmacon RNA Technologies, Lafayette, Colo., USA) was ligated using T4 RNA ligase in the presence of ATP. T4 RNA ligase has a higher ligation efficiency for RNA:RNA ligations, and thus the use of the hybrid DNA:RNA linker inhibited linker self-ligation, and the use of ATP directed ligation to the 5′ monophosphorylated miRNA sequence. Ligation to the 3′ end of the RNA sequences in the reaction mixture was prevented by the presence of the 3′ dideoxy C-containing linker, further directing the ligation reaction to the desired 5′ end of the RNA species, particularly the miRNA species, in the reaction mixture. Full length mRNAs in the reaction mixture were precluded from participating in the 5′ ligation reaction by the presence of the 5′ cap, as were degraded mRNAs by having a 5′ triphosphate which is not a substrate for T4 RNA ligase. Finally, any tRNAs in the mixture are double-stranded at the 5′ end, which inhibits the ligation reaction for those species. rRNAs have extensive secondary structure preventing their ligation and later reverse transcription.

Following linker ligation, the miRNA species were converted to cDNA by reverse transcription using a primer having the sequence: ATT GAT GGT GCC TAC (SEQ ID No: 74) that was complementary to the sequence of the 3′ linker, providing further specificity (Lau et al., 2001, Id.). The resulting cDNA population was amplified by polymerase chain reaction (PCR) using the following primers:

Forward primer:
(SEQ ID NO: 75)
GGC CAG TGA ATT GTA ATA CGA CTC ACT ATA GGG TTC
TCG TGT TCC GTT TGT ACT CTA AGG TGG AAT CGT AGG
CAC CTG AAA
and
Reverse primer:
(SEQ ID NO: 76)
ATT GAT GGT GCC TAC AG.

The forward PCR primer sequence contains three regions: the 3′ region is complementary to the 3′ end of the cDNA, while the 5′ region is a T7 RNA polymerase-specific promoter sequence. In between is a sequence complementary to a “capture” sequence identified as SEQ ID NO: 71 (TTC TCG TGT TCC GTT TGT ACT CTA AGG TGG A). PCR was performed using these primers with one initial denaturation of 95° C. for one minute followed by 20 cycles having a profile of denaturation at 95° C. for 20 seconds, primer annealing at 50° C. for one minute, and primer extension at 72° C. for 30 seconds. There was a final extension step at 72° C. for 5 minutes. The reaction mixture contained 10 units of Taq DNA polymerase in its buffer (as supplied by the manufacturer), 0.2 mM dNTPs, 1.5 mM MgCl₂, 1 μM primers and the reverse transcribed miRNAs obtained in the previous step.

PCR products produced according to these methods were further amplified by using T7 polymerase for in vitro transcription from the T7 promoter sequence in the 5′ forward amplification primer. This provided a “sense”-strand target for hybridization. In addition, this sense-strand reaction product contained a complementary sequence to the “capture sequence”.

The in vitro transcribed sense-strand miRNA-specific products were used as described in the next Example to interrogate a microarray comprising antisense miRNA probes in order to identify miRNA species expressed or overexpressed in NPC tumors.

### Example 2

### Microarray Construction and Hybridization

The in vitro transcribed sense-strand miRNA-specific products prepared according to Example 1 were used to interrogate a microarray comprising antisense miRNA probes as follows.

Microarrays were prepared comprising probes that were antisense dimers of mature miRNA sequences taken from miRBase (microrna.sanger.ac.uk/), previously termed “the microRNA registry” (Griffiths-Jones, 2004, The microRNA Registry Nucl. Acids. Res. 32: Database Issue, D109-D111). Each miRNA probe sequence used in the microarray was modified at its 5′ end with a C6 amino linker that permitted the probe to be attached to aldehyde-coated slides for microarray fabrication. A total of two hundred seven probes from two hundred twenty-two human miRNAs and six probes for five EBV miRNAs (as present in the database as of April 2005) were spotted on a chip. Also spotted were seven probes from D. melanogaster miRNAs as controls (Table 1). Microarrays were printed in quadruplicate for each probe in an amount of 40 μM probe in 2.4×SSC on aldehyde-coated slides (ArrayIt SuperAldehyde Substrates, obtained from Telechem International, Inc., Sunnyvale, Calif., USA) using a BioRobotics MicroGrid II microarrayer (Genomic Solutions, Ann Arbor, Mich., USA). The microarrays were preprocessed according to the slide manufacturer's instructions.

Two hybridization steps were performed on these arrays: 1) sense target hybridization, and 2) capture sequence hybridization (illustrated in FIG. 1). For the first hybridization, in vitro transcribed sense targets were hybridized to the microarrays overnight at 55° C. under LifterSlips (Thermo Fisher Scientific Inc., N.H., USA) inside humidified hybridization chambers according to the manufacturer's instructions (26 μl hybridization volume, ˜50 μg of product, and SDS-based hybridization buffer included in the kit).

After hybridization, the arrays were washed, spin-dried and the second hybridization was performed to detect the position in the array that had hybridized to an amplified miRNA species in the hybridization mixture. The washing condition used for both washes follows: (a) removed the LifterSlip by putting the array in a beaker containing 2×SSC, 0.2% SDS, where the solution being at 55° C. for the first hybridization and 42° C. for the second hybridization; (b) washed for 15 minutes in 2X SSC, 0.2% SDS; (c) then washed for 15 minutes in 2×SSC; (d) and then finally washed for 15 minutes in 0.5×SSC.

The second hybridization used a Cy3 3DNA molecule containing the “capture sequence” wherein these molecules contained an aggregate of approximately 900 fluorophores; these reagents and buffers were commercially available (34 μl vol containing 2.5 μl of 3DNA capture reagent, 14.5 μl water and 17 μl SDS-based hybridization buffer) (3DNA Array 900 Microarray detection kit, Genisphere Inc., Hatfield, Pa., USA). After the second hybridization at 42° C. for 4 hours, the arrays were again washed (conditions above), dried and scanned. Data was acquired with GenePix Pro 5.0 (Molecular Devices, Sunnyvale, Calif., USA). All hybridization buffers, wash conditions etc. used in the second detection reaction were provided by/according to Genisphere. The results of these assays, and further characterization of the miRNA species, are presented in Example 3.

### Example 3

### Identification of Differentially Expressed miRNAs

Cellular and viral miRNAs in EBV-associated cancers such as NPC are candidate oncogenes that may contribute to the initiation or maintenance, or both, of tumors. Accordingly, the microarray methods described above were used to screen a large number of cellular and viral miRNAs for differential expression in NPC tumors. These assays were performed using microarrays prepared as described in Example 2, comprising two hundred twenty-two human miRNAs and for five viral miRNAs, which included all miRNAs identified as of April 2005. These assays were performed substantially as described above.

The results of these assays are given in Table 2. In these experiments, background-corrected, raw-scale expression intensity values were obtained via GenePix Pro 5.0 (Molecular Devices) after some manual adjustment to align and identify spots. Data from multiple microarrays were normalized using a version of quantile normalization (Bolstad et al., 2003, Bioinformatics 19:185-93) in which the expression value at the pth quantile on the ith microarray was replaced by the median of pth quantiles across the set of all 41 microarrays. Gene-specific hypothesis tests were applied to the quantile-normalized data in order to assess differential expression between tumor and normal microRNA profiles. To minimize false positive calls and retain robustness, multiple statistical tests (including Wilcoxon rank sum, t-test, raw scale, and t-test, log scale at 5% false discovery rate) were used to establish the significance of the differences in expression between tumor and normal tissue. In applying this statistical analysis, an miRNA species was determined to be differentially expressed if it was significantly different by all three tests, at the 5% false discovery rate:. Gene-specific p-values were converted to q-values (Storey and Tibshirani, 2003, Proc Natl Acad Sci U S A. 100:9440-5); the list containing genes with q-value <=5% is expected to have no more than 5% false positives.

For the miRNA results, robust differential expression was detected between tumor and normal tissues; in these analyses miRNAs expressed at very low levels, less than 800 relative fluorescence units (RFUs), in both tissue types were excluded from the analysis. Eight miRNAs showed a greater than five-fold differential in expression between normal and tumor tissues. Of these, six miRNAs (miR-29c, miR-34c, miR-34b, miR-212, miR-216 and miR-217) showed significantly higher expression in normal cells as compared to tumors and 2 (miR-151 and miR-192) showed significantly higher expression in tumors as compared to normal samples in this analysis (Table 3).

TABLE 3
miRNAs differentially expressed between normal and NPC tumor tissues

| | Normal* | Tumor* | Fold difference | Wilcoxon |
|---|---|---|---|---|
| miRNA | (n = 10) | (n = 31) | (Tumor/Normal) | p-value** |
| miR-29c | 32320 | 6567 | 0.20 | 0.002 |
| miR-34b | 28879 | 3252 | 0.11 | 0.000 |
| miR-34c | 25243 | 1461 | 0.06 | 0.001 |
| miR-212 | 4363 | 885 | 0.20 | 0.000 |
| miR-216 | 6843 | 940 | 0.14 | 0.002 |
| miR-217 | 4212 | 351 | 0.08 | 0.000 |
| miR-151 | 60 | 3598 | 60.25 | 0.001 |
| miR-192 | 71 | 1573 | 22.02 | 0.000 |

*Each miRNA level is reported as the median of miRNA expression levels (microarray-normalized probe fluorescence) for all (n = 10) normal or (n = 31) tumor samples respectively
**Probability that a particular miRNA is not differentially expressed, based on will cover rank sum comparison of all 310 possible tumor normal pairs. Wilcoxon, F. “Individual Comparisons by Ranking Methods,” Biometrics 1, 80-83, 1945.

Hence stringent statistical criteria established eight human miRNAs to be differentially expressed between tumor and normal tissues.

### Example 4

### Identification of Target mRNAs

The results shown in Example 3 identified eight human miRNAs that were significantly differentially expressed between normal and tumor tissues and that likely contribute to tumor phenotype. The assays described in this Example were performed to identify mRNA species whose expression is regulated by any of these eight miRNAs.

These assays were performed by applying two algorithms, both of which predicted targets by finding sequences in 3′ UTRs of mRNAs that match nucleotides 2 through 7 of the 5′ end of the identified miRNAs. The first, termed PicTar (Krek et al., 2005, Nat Genet. 37:495-500) also further refined its predictions by searching for target conservation in mammals (human, chimp, mouse, rat, dog) (pictar.bio.nyu.edu/cgi-bin/PicTar_vertebrate.cgi). The second algorithm, termed TargetScan (Lewis et al., (2003, Cell. 115:787-98), looked for conservation of target sites in vertebrates (www.targetscan.org). Targets predicted by both algorithms were considered in further analysis.

The target sites of miRNAs in mRNAs often are evolutionarily conserved and considering such conservation increases the reliability of identifying targets (Lewis et al., 2005, Cell. 120:15-20). Because these target sites are identified by a minimum perfect complementarity of only 7 to 8 nucleotides at the 5′ end of the miRNAs (the ‘seed’ sequence), these algorithms sometimes produce false-positive targets. In addition to regulating gene expression by inhibiting translation (which is thought to be the more common action of miRNAs), miRNAs can also regulate expression of a subset of their targets by decreasing mRNA stability (Yekta et al., Science. 304:594-596; Bagga et al., 2005, Cell. 122:553-563; and Wu et al., 2006, Proc Natl Acad Sci USA. 103:4034-4039). Such miRNA function should be evident in gene expression profiling data. Therefore, prior mRNA profiling (Sengupta et al., 2006, Cancer Res. 66:7999-8006) results were used to find bona fide targets among the large number of predicted target mRNAs of the eight highly differentially expressed miRNAs, by identifying those targets that accumulate differentially between tumor and normal samples.

None of the predicted target mRNAs for mir-151 and mir-192 showed differential mRNA accumulation. However, statistically significant differentially accumulating, candidate target mRNAs for the six miRNAs whose levels decreased in NPC were identified (Table 4). The largest set of differentially expressed predicted targets was associated with mir-29c. Mir-29c levels averaged one-fifth the level in NPC tumors as in normal nasopharyngeal epithelium (Table 3) and, correspondingly, the 15 differentially accumulating, predicted mir-29c target mRNAs accumulated to 2- to 6-fold higher levels in NPC tumors (Table 4). Strikingly, 10 of these 15 differentially accumulating candidate target mRNAs of mir-29c were involved in extracellular matrix synthesis or its functions, including 7 collagens, laminin yl, fibrillin, and SPARC (secreted protein, acidic, cysteine-rich). Interestingly, two differentially expressed mir-29c targets, laminin γl and FUSIP1 (FUS interacting protein) mRNAs, also were predicted targets of mir-216 and mir-21 7, respectively, which like mir-29c were downregulated miRNAs in NPC tumors (Tables 3 and 4).

The seed sequence of mir-29c is identical to that of its two family members, mir-29a and mir-29b. These three mir-29 species vary in their last few 3′-end nucleotides. In addition, in close proximity to its seed sequence, mir-29a has a single nucleotide difference from mir-29b&c, giving mir-29c an overlapping but distinct list of predicted target mRNAs. Mir-29a is expressed at slightly higher levels than mir-29c in normal tissue, and its levels are moderately decreased in tumors. Mir-29b, predominantly targeted to the nucleus (Hwang et al., 2007, Science. 315:97-100), is expressed at one-fourth the level of mir-29c in normal nasopharyngeal epithelium. In NPC tumors, mir-29b and mir-29c have similar 4-fold to 5-fold decreased levels (Table 2). Thus, the levels of all three mir-29 family members are decreased in tumors, implying parallel effects on their shared targets. The mechanism of action of miRNA-mediated gene expression regulation is understood to encompass not only modulating mRNA translation. This miRNA-mediated gene expression regulation may include, for example, decreasing mRNA translation or reducing stability of specific mRNAs (Yekta et al., 2004, Science. 304:594-6; Wu et al., 2006, Proc Natl Acad Sci USA. 103:4034-9). Thus, all predicted targets for these 8 miRNAs were cross checked for differential expression between NPC tumor samples and corresponding normal tissues (Sengupta et al., 2006, Cancer Res. 66: 7999-8006) to identify mRNAs that are downregulated in tissue (tumor/normal) where the miRNA is upregulated. Excluded from analysis were those mRNAs detected at low levels in both tumor and normal cells, to insure that only robust potential targets were considered. Target mRNAs for six of the eight miRNAs were found which showed downregulation in tissues where the miRNA was upregulated (Table 4). One miRNA, miR-29c had a group of target genes that were functionally related.

For many tumor cells, increased extracellular levels of collagens and/or laminins have been shown to induce increased invasiveness in culture and increased metastasis in animal models (Kaufman et al., 2005, Biophys 1 89:635-650; Koenig et al., 2006 Cancer Res. 66:4662-4671; Chintala et al., 1996, Cancer Lett 102:57-63; Kuratomi et al., 1999, Exp Cell Res. 249:386-395; Kuratomi et al., 2002, Br J Cancer. 86:1169-1173; Song et al., 1997, Int J. Cancer. 71:436-441; Menke et al., 2001, Cancer Res. 61:3508-3517;

Shintani et al., 2006, Cancer Res 66:11745-11753). Similarly, increased levels of collagens and laminins have been associated with an increased likelihood of clinical metastasis of multiple human solid tumors (Ramaswamy et al., 2003, Nat Genet 33:49-54). The results set forth herein, disclosing use of laser-capture to isolate tumor cells essentially free of stromal contaminants (Sengupta et al., 2006, Cancer Res. 66:7999-8006). indicated that NPC tumor cells upregulate mRNAs encoding collagens and laminins.

TABLE 4
Fold Changes in miRNA targeted mRNAs

| | | Fold Change |
|---|---|---|
| miRNA | Target mRNA | (Tumor/Normal) |
| miR-29c | FLJ12505 | 6.34 |
| miR-29c | COL4A1 | 5.24 |
| miR-29c | COL4A2 | 4.58 |
| miR-29c | COL3A1 | 4.14 |
| miR-29c | COL1A2 | 4.10 |
| miR-29c | COL5A2 | 4.05 |
| miR-29c | FBN1 | 2.98 |
| miR-29c | SPARC | 2.93 |
| miR-29c | COL15A1 | 2.92 |
| miR-29c | FUSIP1 | 2.59 |
| miR-29c | COL1A1 | 2.31 |
| miR-29c | TFEC | 2.27 |
| miR-29c | IFNG | 2.24 |
| miR-29c | LAMC1 | 2.06 |
| miR-29c | TDG | 1.80 |
| miR-34b&c | CCNE2 | 4.52 |
| miR-34b&c | ATP11C | 3.55 |
| miR-34b&c | IQGAP3 | 3.14 |
| miR-34b&c | SOX4 | 2.77 |
| miR-34b&c | ARNT2 | 2.27 |
| miR-34b&c | VEZATIN | 2.07 |
| miR-34b&c | E2F3 | 2.05 |
| miR-212 | SOX4 | 2.77 |
| miR-212 | EIF2C2 | 1.64 |
| miR-216 | LAMC1 | 2.06 |
| miR-216 | NFYB | 1.85 |
| miR-217 | FN1 | 7.39 |
| miR-217 | ANLN | 3.70 |
| miR-217 | EZH2 | 2.74 |
| miR-217 | FUSIP1 | 2.59 |
| miR-217 | POLG | 2.57 |
| miR-217 | DOCK4 | 2.48 |
| miR-217 | HNRPA2B1 | 1.63 |

Fold change was averaged for mRNAs that were detected by multiple probes

### Example 5

### Transfections and Quantitative Real Time PCR Analysis

The capacity of the miRNA species miR-29c to regulate the target mRNAs identified above was confirmed as follows.

A precursor of miR-29c was introduced into human epithelial and liver cell lines Hela and HepG2 and the levels of the processed miRNA and its target mRNAs were assayed by quantitative real time PCR. The resulting changes in levels of the mature miRNA and its target mRNAs relative to their levels in untransfected cells were measured (Table 5). HeLa and HepG2 were transfected with miR-29c precursor molecules and negative controls (Ambion, Austin, Tex., USA) using TranslT-TKO reagent (Minis Bio Corporation, Madison, Wis., USA). Transfection efficiencies were monitored with LabelIT miRNA Labeling Kit (Minis Bio Corporation, Madison, Wis., USA). Levels of mature miR-29c in transfected and untransfected control cells were measured by stem-loop quantitative PCR (Chen et al., 2005, Nucleic Acids Res. 33:179) using TaqMan MicroRNA Assay and TaqMan MicroRNA Reverse Transcription Kits (Applied Biosystems, Foster City, Calif., USA). mRNA from untransfected cells and cells transfected with the negative control and miR-29c precursor were reverse transcribed using oligo-dT primers and SuperScript™ II Reverse Transcriptase (Invitrogen, Carlsbad, Calif., USA) and expression of miR-29c target genes was measured by quantitative real time PCR using QuantiTect SYBR Green PCR Kit (Qiagen, Valencia, Calif., USA). The primer sequences are listed in Table 6. All experimental manipulations disclosed in this Example were performed according to the manufacturers' instructions and as understood by one having skill in this art. All gene measurements were done 24 h post-transfection.

The transfected Hela and HepG2 cells had a 100- and 10-fold increase in their level of mature mirR-29c, respectively, as measured by stem loop quantitative real time PCR relative to untransfected cells or those transfected with a negative control precursor RNA that is processed into a randomized sequence not matching any known miRNA. In HeLa cells, 8 potential miR-29c target mRNAs were detected at high copy numbers. Another five (collagen 3A1, 4A1, 15A1, laminin γl and thymine-DNA glycosylase (TDG)) were reduced significantly by miR-29c transfection, as shown in FIG. 3 and Table 5. In HepG2 cells, reductions were seen for 4 of these 5 mRNAs (the fifth, collagen 3A1 mRNA, was not detectable above background levels).

In addition, HepG2 cells showed significant, above-background measurements for additional miR-29c candidate targets collagen 1A2, fibrillin 1, SPARC and FUSIP1 mRNAs, revealing miR-29c-mediated reductions for all of those except SPARC (FIG. 3 and Table 5). In all cases, these miR-29c-induced reductions were much greater than any increases or decreases induced by parallel transfection of the randomized, negative control precursor miRNA, showing that the observed downregulation of these mRNA species was miRNA sequence-specific. In particular, introducing the miRNAs into HeLa or HepG2 cells did not elicit an interferon response, as there were no significant changes in expression of mRNAs for interferon-activated genes STAT1 and OAS1 (data not shown). In addition, all control or miR-29c-transfected cultures had similar levels of GAPDH mRNA, an mRNA lacking target homology to miR-29c. Sequences of primers used to carry out real time PCR measurements of these genes are listed in Table 6.

TABLE 5
GADPH normalized mir-29c candidate target gene expression
in HeLa and HepG2 cells

| | Fold | | | | | | |
|---|---|---|---|---|---|---|---|
| | Change | Mean mRNA levels | | | Fold Change | | |
| | in | | Negative | | (Untransfected/ | | |
| Target | Tumor/ | | control- | mir-29c- | mir-29c- | Fold Change | |
| mRNAs | Normal | Untransfected | transfected | transfected | transfected) | t statistic | p value |
| | | HeLa Cells | | | | | |
| COL4A1 | 5.2 | 1430.8 | 1001.8 | 656.4 | 2.2 | 9.48 | 0.00 |
| COL15A1 | 2.9 | 2574.7 | 2287.2 | 1252.0 | 2.1 | 7.49 | 0.03 |
| COL1A1* | 2.3 | 2110.0 | 3228.6 | 2544.5 | 0.8 | −1.32 | 0.86 |
| COL1A2* | 4.1 | | | | | | |
| COL3A1* | 4.1 | 2657.4 | 2106.5 | 693.7 | 3.8 | 11.65 | 0.00 |
| COL4A2* | 4.6 | 1873.2 | 1855.6 | 2229.1 | 0.8 | −1.13 | 0.81 |
| LAMC1 | 2.1 | 1781.7 | 1203.7 | 863.4 | 2.1 | 11.74 | 0.00 |
| TDG | 1.8 | 2661.9 | 2618.3 | 1456.4 | 1.8 | 6.05 | 0.00 |
| FBN1* | 3.0 | | | | | | |
| SPARC* | 2.9 | | | | | | |
| FUSIP1 | 2.6 | 3146.0 | 3467.4 | 3889.6 | 0.8 | −8.00 | 1.00 |
| OAS1** | 1.0 | 41.7 | 37.8 | 43.3 | 0.9 | | |
| | | HepG2 Cells | | | | | |
| COL4A1 | 5.2 | 30.9 | 17.1 | 3.0 | 10.3 | 2.55 | 0.06 |
| COL15A1* | 2.9 | 60.0 | 78.5 | 2.0 | 29.5 | 4.32 | 0.02 |
| COL1A1* | 2.3 | | | | | | |
| COL1A2* | 4.1 | 189.8 | 37.4 | 9.8 | 19.4 | 1.34 | 0.16 |
| COL3A1* | 4.1 | | | | | | |
| COL4A2* | 4.6 | | | | | | |
| LAMC1 | 2.1 | 334.9 | 344.7 | 218.4 | 1.5 | 1.16 | 0.16 |
| TDG | 1.8 | 590.5 | 910.8 | 209.0 | 2.8 | 2.19 | 0.07 |
| FBN1* | 3.0 | 400.9 | 359.5 | 13.4 | 29.9 | 2.53 | 0.06 |
| SPARC* | 2.9 | 224.4 | 462.2 | 208.7 | 1.1 | 0.40 | 0.36 |
| FUSIP1 | 2.6 | 1337.5 | 2618.8 | 930.1 | 1.4 | 1.61 | 0.11 |
| OAS1** | 1.0 | 29.9 | 27.9 | 38.7 | 0.8 | | |

mRNA accumulation in tissue culture cells was measured by quantitative real time PCR, normalized to GADPH mRNA accumulation were measured in triplicate except for the untransfected and negative control for HeLa, which were measured in duplicate and once for OAS1
For mRNA detected by multiple probes, fold changes (tumors/normals) were averaged.
*Measurements were left blank for these mRNAs in the cell line where they were not detected above background levels
**OAS1 is not a mir-29c candidate target gene

TABLE 6
Primers used for Quantitative Real Time PCR

| Gene | Forward Primer (5′-3′) | Reverse Primer (5′-3′) |
|---|---|---|
| COL1A1 | CCCAAGGACAAGAGGCATGT | CCGCCATACTCGAACTGGAA |
| | (SEQ ID NO: 505) | (SEQ ID NO: 506) |
| COL1A2 | GATTGAGACCCTTCTTACTCCTGAA | GGGTGGCTGAGTCTCAAGTCA |
| | (SEQ ID NO: 507) | (SEQ ID NO: 508) |
| COL3A1 | TGGACAGATTCTAGTGCTGAGAAGA | TTGCCGTAGCTAAACTGAAAAC |
| | (SEQ ID NO: 509) | C (SEQ ID NO: 510) |
| COL4A1 | GTATTTTCACACGTAAGCACATTCG | CCCTGCTGAGGTCTGTGAACA |
| | (SEQ ID NO: 511) | (SEQ ID NO: 512) |
| COL4A2 | GTGGCCAATCACTGGTGTCA | CCTCCATTGCATTCGATGAA |
| | (SEQ ID NO: 513) | (SEQ ID NO: 514) |
| COL5A1 | CCCCGATGGCTCGAAAA | TGCGGAATGGCAAAGCTT |
| | (SEQ ID NO: 515) | (SEQ ID NO: 516) |
| COL15A1 | CTCGTACCTCAGCATGCCATT | GCCTTCACTGTCCAGGATCAG |
| | (SEQ ID NO: 517) | (SEQ ID NO: 518) |
| FBN1 | GCCCCCTGCAGCTATGG | GGCCTATGCGGAAGTAACCA |
| | (SEQ ID NO: 519) | (SEQ ID NO: 520) |
| FLJ12505 | GGAAAAGTCTTCGGTCCAGTGT | TATGCAGGCCAGACATTCATTC |
| | (SEQ ID NO: 521) | (SEQ ID NO: 522) |
| FUSIP1 | CCCCCCAACACGTCTCTG | TCACGCCGCAAGTCTTCAG |
| | (SEQ ID NO: 523) | (SEQ ID NO: 524) |
| IFNG | CCAACGCAAAGCAATACATGA | TTTTCGCTTCCCTGTTTTAGCT |
| | (SEQ ID NO: 525) | (SEQ ID NO: 526) |
| LAMC1 | TTGACGCCACAGTGGGACTA | CAGCTCCAACAATTGCCAAA |
| | (SEQ ID NO: 527) | (SEQ ID NO: 528) |
| OAS1 | CTGACGCTGACCTGGTTGTCT | CCCCGGCGATTTAACTGAT |
| | (SEQ ID NO: 529) | (SEQ ID NO: 530) |
| SPARC | CACATTAGGCTGTTGGTTCAAACT | CAGGATGCGCTGACCACTT |
| | (SEQ ID NO: 531) | (SEQ ID NO: 532) |
| STAT1 | TCATCTGTGATTCCCTCCTGCTA | GCTGGCCTTTCTTTCATTTCC |
| | (SEQ ID NO: 533) | (SEQ ID NO: 534) |
| TDG | TGCACACTCAGACCTCTTTGCT | TGTCAGGTAAGGGCCAGTTTTT |
| | (SEQ ID NO: 535) | (SEQ ID NO: 536) |
| GAPDH | TCAACGACCACTTTGTCAAGCT | CCATGAGGTCCACCACCCT |
| | (SEQ ID NO: 537) | (SEQ ID NO: 538) |

### Example 6

### Mir-29c Regulation of Target Gene Expression

To verify mir-29′s regulation of target gene expression, 3′ UTRs containing mir-29c binding site sequence, were cloned into expression vectors containing a luciferase reporter gene. Specifically, 10 mir-29c target gene 3′ UTRs were cloned into a vector immediately downstream of a firefly luciferase gene. As a control, the GAPDH 3′UTR, which is not a mir-29c target, was cloned downstream of luciferase.

The firefly luciferase expression vector pGL2-control (Promega, Madison, Wis.) was modified by introducing silent mutations in a potential mir-29c binding sequence in the firefly luciferase ORF (nt positions 844-860) and by replacing the 3′UTR of the luciferase gene with a double stranded oligonucleotide linker to create a multiple cloning site (NotI-SpeI-Pstl-BamHI-Sa/I) immediately downstream from the Firefly luciferase ORF, while removing the existing Sa/I site from the original plasmid. This new vector, pJBLuc3UTR (SEQ ID NO: 539), accommodated subsequent insertion of the entire 3′UTR sequences of 12 mRNAs:, COL1A1 (SEQ ID NO: 540), COL1A2 (SEQ ID NO:

541), COL3A1 (SEQ ID NO: 542), COL4A1 (SEQ ID NO: 543), COL4A2 (SEQ ID NO: 544), COL15A1 (SEQ ID NO: 545), FUSIP1 isoform 1 (SEQ ID NO: 546) and 2 (SEQ ID NO: 547), GAPDH (SEQ ID NO: 548), LAMC1 (SEQ ID NO: 549), SPARC (SEQ ID NO: 550), and TDG (SEQ ID NO: 551). Full sequences are also provided for reference: COL1A1 (SEQ ID NO: 552), COL1A2 (SEQ ID NO: 553), COL3A1 (SEQ ID

NO: 554), COL4A1 (SEQ ID NO: 555), COL4A2 (SEQ ID NO: 556), COL15A1 (SEQ ID NO: 557), FUSIP1 isoform 1 (SEQ ID NO: 558) and 2 (SEQ ID NO: 559), GAPDH (SEQ ID NO: 560), LAMC1 (SEQ ID NO: 561), SPARC (SEQ ID NO: 562), and TDG (SEQ ID NO: 563). (See Appendix 1 for the above-mentioned sequences). The 3′UTR sequences were PCR-amplified from oligo-d(T)-primed HeLa cDNA derived from 10 total RNA extracted using RNeasy reagents and protocol (Qiagen, Valencia, CA). cDNA was generated using the SuperScrip™II cDNA synthesis kit (Invitrogen, Carlsbad, Calif.) according to instructions. PCRs contained a mixture of 0.25U Vent DNA polymerase (New England Biolabs, Ipswich, Mass.) and 1.875U Taq DNA polymerase (Promega, Madison, Wis.) in a 50 μl 133 Vent DNA polymerase buffer system supplemented with 1.5 mM MgCl₂, 1 ng template plasmid, 100 μM of all four dNTPS and 25 pmoles of each of two primers. Upon 5 minutes denaturation at 95° C., 30 amplification cycles were used (1 min 95° C.-30 sec 55° C.-1 min/kbp 72° C.) followed by 10 min at 72° C. and refrigeration to 4° C. PCR-primers were designed to introduce Spel or Nhel-sites and Sall sites immediately upstream and downstream from the mRNA specific sequences, respectively, to facilitate subcloning between the SpeI and Sa/I sites of the modified luciferase expression vector using standard molecular biology procedures. Reporter plasmids for COL1A1, COL3A1, and COL4A2 3′UTRS then served as templates for PCR-mediated mutagenesis of all multiple mir-29c target sequences (FIG. 5A) using amplification conditions as described above. All PCR-derived sequence elements were sequenced using Big Dye chemistry (Applied Biosystems, Foster City, Calif.) according to manufacturer's instructions and analyzed at the University of Wisconsin-Madison Biotech Center's core sequencing facilities.

The reporter plasmids described above were transfected into HeLa cell using TranslT-HeLaMONSTER transfection reagents and conditions from Minis Bio Corporation (Madison, Wis.). 1.2×10⁶ HeLa cells were co-transfected with 500 ng Firefly reporter plasmids and 250 ng internal reference Renilla luciferase reporter plasmid pRL-SV40 (Promega, Madison, Wis.) in a final transfection volume of 1050 μl. At 4 hours post plasmid transfection, culture medium was removed and cells were mock-transfected or transfected with 25 pmoles mir-29c precursor (Ambion, Austin, Tex.) using TransIT-TKO reagents under conditions recommended by the manufacturer (Minis Bio Corporation, Madison, Wis.) at a final transfection volume of 600 μl. Lysates were prepared at 24 hours post-transfection.

For dual luciferase reporter assays, transfected cells were lysed in 200 μl “passive lysis buffer” (Promega, Madison, Wis.) for 10 min at room temperature, scraped, resuspended, and cleared of nuclei and large cell debris by centrifugation at 10,000×g for 2 min at 4° C. Lysates were stored at −80° C. prior to analysis. 15 μl aliquots of the lysates were analyzed for Firefly luciferase activity and subsequently for Renilla luciferase activity using the Promega “Dual Luciferase Assay kit” for combined Firefly and Renilla luciferase assays as per accompanying instructions. Enzymatic activities were measured by luminometry using a Wallac 1420 Multilabel Counter (Victor3™V, Perkin Elmer, Waltham, Mass.). All measurements were normalized for Renilla luciferase activity to correct for variations in transfection efficiencies and non-mir-29c-specific effects of miRNA transfection on enzymatic activity.

For the experimental studies represented in FIGS. 4 and 5, HeLa cells were transfected with the mir-29c target gene 3′ UTR/luciferase constructs with or without subsequent mir-29c precursor RNA transfection. The 3′ UTRs of all of these 10 candidate target genes (Collagen 1A1, 1A2, 3A1, 4A1, 4A2, 15A1, FUSIP1isol, laminin γl, SPARC and TDG) elicited significantly decreased luciferase activities (p values from 3×10⁻³ to 1.2×10⁻⁷) in mir-29c transfected cells (FIG. 4). These inhibitions, ranging from −20-50%, are similar in magnitude to equivalent experiments involving transfection of miRNA precursors (Mott et al., 2007, Oncogene. 26:6133-6140; Fabbri et al., 2007, Proc Natl Acad Sci USA. 104:15805-15810). In general, for each 3′ UTR, mir-29c-induced reductions in luciferase activity (FIG. 4) correlated well with the magnitude of the mir-29c-induced reduction in the level of the corresponding complete mRNA (FIG. 3). These findings with FUSIP1 provide additional support for the specificity of mir-29c inhibition. FUSIP1 has two isoforms and only one of them (isoforml) is a potential target for mir-29c. The 3′ UTR of isoform2 did not support detectable inhibition of luciferase activity by mir-29c while that of isoforml led to statistically significant inhibition (p value=3×10⁻³) (FIG. 3).

The magnitude of the mir-29c effects reported here for target mRNAs (FIG. 4), ranging from ˜20-50% inhibition, is consistent with the effects of transfecting other single miRNAs (Mott et al., 2007, Oncogene. 26:6133-6140; Fabbri et al., 2007, Proc Natl Acad Sci USA. 104:15805-15810). Frequently, multiple miRNAs can target a single mRNA, thus increasing their effectiveness (Grimson et al. 2007, Mol Cell. 27:91-105). For example, in neuroblastoma cells, three different miRNAs regulate the levels of a single protein (Laneve et al., 2007, Proc Natl Acad Sci USA. 104:7957-7962). Similarly, two differentially expressed mir-29c targets, laminin γl and FUSIP1 mRNAs, are also predicted targets of mir-216 and mir-217, respectively, which like mir-29c were downregulated in NPC tumors. Moreover, in addition to downregulating mRNA accumulation, the same miRNA(s) may inhibit translation of their target RNAs.

Nucleotide substitutions disrupting the mir-29c binding site(s) were introduced in the 3′ UTRs of collagen 1A1, 3A1, and 4A2 cloned downstream of the firefly luciferase gene (FIG. 5A). In every case, this disruption of the target binding-sites for mir-29c abrogated the inhibition of luciferase activity by mir-29c (FIG. 5B). Thus, the predicted target sequences were responsible for the mir-29c-sensitivity of these 3′UTRs.

In summary, miRNA expression profiling was performed in laser-microdissected NPC and normal surrounding epithelial cells using a sensitive assay specifically developed to detect miRNA expression from small samples limited in the amount of source tumor cells, the amount of miRNA or both. Eight of 207 assayed miRNAs displayed >5 fold differential expression levels in NPC cells compared to surrounding normal epithelium (Table 3). Using bioinformatic approaches candidate target genes of these 8 miRNAs were identified. Next, mRNA expression profiling was performed on these same specimens (Sengupta et al., 2006, Cancer Res. 66:7999-8006) further identifying candidate target genes that were differentially expressed, likely due to action of these miRNAs. Among the differentially expressed candidate target genes of the 8 miRNAs, those of mir-29c showed a group of 15 genes, 10 of which were extracellular matrix components involved in cell migration and metastasis (Table 4). In tumor cells, mir-29c levels were decreased >5 fold whereas these mRNAs were upregulated 2- to 6-fold.

Using multiple tissue culture-based assays (FIG. 3-5), the regulation of these candidate target genes by mir-29c was verified. Transfection and reporter assays confirmed regulation of 11 target genes by mir-29c. The results illustrate that the reduced levels of mir-29c in NPC tumors allowed the observed increase in mRNA levels of multiple extracellular matrix components, which as noted before would facilitate rapid matrix generation and renewal during tumor growth and the acquisition of tumor motility.

All references cited herein are incorporated by reference. In addition, the invention is not intended to be limited to the disclosed embodiments of the invention. It should be understood that the foregoing disclosure emphasizes certain specific embodiments of the invention and that all modifications or alternatives equivalent thereto are within the spirit and scope of the invention as set forth in the appended claims.

TABLE 1
Probes used in the miRNA Microarray

| miRNA/Probe Nam | 5′-3′ Mature miRNA Sequence | 5′-3′ Probe Sequence |
|---|---|---|
| let-7a | tgaggtagtaggttgtatagtt (SEQ ID NO: 77) | aactatacaacctactacctcaaactatacaacctactacctca (SEQ ID NO: 78) |
| let-7b | tgaggtagtaggttgtgtggtt (SEQ ID NO: 79) | aaccacacaacctactacctcaaaccacacaacctactacctca (SEQ ID NO: 80) |
| let-7c | tgaggtagtaggttgtatggtt (SEQ ID NO: 81) | aaccatacaacctactacctcaaaccatacaacctactacctca (SEQ ID NO: 82) |
| let-7d | agaggtagtaggttgcatagt (SEQ ID NO: 83) | actatgcaacctactacctctactatgcaacctactacctct (SEQ ID NO: 84) |
| let-7e | tgaggtaggaggttgtatagt (SEQ ID NO: 85) | actatacaacctcctacctcaactatacaacctcctacctca (SEQ ID NO: 86) |
| let-7f | tgaggtagtagattgtatagtt (SEQ ID NO: 87) | aactatacaatctactacctcaaactatacaatctactacctca (SEQ ID NO: 88) |
| let-7g | tgaggtagtagtttgtacagt (SEQ ID NO: 89) | actgtacaaactactacctcaactgtacaaactactacctca (SEQ ID NO: 90) |
| let-71 | tgaggtagtagtttgtgctgt (SEQ ID NO: 91) | acagcacaaactactacctcaacagcacaaactactacctca (SEQ ID NO: 92) |
| miR-1 | tggaatgtaaagaagtatgta (SEQ ID NO: 93) | tacatacttctttacattccatacatacttctttacattcca (SEQ ID NO: 94) |
| miR-7 | tggaagactagtgattttgttg (SEQ ID NO: 95) | caacaaaatcactagtcttccacaacaaaatcactagtcttcca (SEQ ID NO: 96) |
| miR-9 | tctttggttatctagctgtatga (SEQ ID NO: 97) | tcatacagctagataaccaaagatcatacagctagataaccaaaga (SEQ ID NO: 98) |
| miR-9* | taaagctagataaccgaaagt (SEQ ID NO: 99) | actttcggttatctagctttaactttcggttatctagcttta (SEQ ID NO: 100) |
| miR-10a | taccctgtagatccgaatttgtg (SEQ ID NO: 101) | cacaaattcggatctacagggtacacaaattcggatctacagggta (SEQ ID NO: 102) |
| miR-10b | taccctgtagaaccgaatttgt (SEQ ID NO: 103) | acaaattcggttctacagggtaacaaattcggttctacagggta (SEQ ID NO: 104) |
| miR-15a | tagcagcacataatggtttgtg (SEQ ID NO: 105) | cacaaaccattatgtgctgctacacaaaccattatgtgctgcta (SEQ ID NO: 106) |
| miR-15b | tagcagcacatcatggtttaca (SEQ ID NO: 107) | tgtaaaccatgatgtgctgctatgtaaaccatgatgtgctgcta (SEQ ID NO: 108) |
| miR-16 | tagcagcacgtaaatattggcg (SEQ ID NO: 109) | cgccaatatttacgtgctgctacgccaatatttacgtgctgcta (SEQ ID NO: 110) |
| miR-17-3p | actgcagtgaaggcacttgt (SEQ ID NO: 111) | acaagtgccttcactgcagtacaagtgccttcactgcagt (SEQ ID NO: 112) |
| miR-17-5p | caaagtgcttacagtgcaggtagt (SEQ ID NO: 113) | actacctgcactgtaagcactttgactacctgcactgtaagcactttg (SEQ ID NO: 114) |
| miR-18 | taaggtgcatctagtgcagata (SEQ ID NO: 115) | tatctgcactagatgcaccttatatctgcactagatgcacctta (SEQ ID NO: 116) |
| miR-19a | tgtgcaaatctatgcaaaactga (SEQ ID NO: 117) | tcagttttgcatagatttgcacatcagttttgcatagatttgcaca (SEQ ID NO: 118) |
| miR-19b | tgtgcaaatccatgcaaaactga (SEQ ID NO: 119) | tcagttttgcatggatttgcacatcagttttgcatggatttgcaca (SEQ ID NO: 120) |
| miR-20 | taaagtgcttatagtgcaggtag (SEQ ID NO: 121) | ctacctgcactataagcactttactacctgcactataagcacttta (SEQ ID NO: 122) |
| miR-21 | tagcttatcagactgatgttga (SEQ ID NO: 123) | tcaacatcagtctgataagctatcaacatcagtctgataagcta (SEQ ID NO: 124) |
| miR-22 | aagctgccagttgaagaactgt (SEQ ID NO: 125) | acagttcttcaactggcagcttacagttcttcaactggcagctt (SEQ ID NO: 126) |
| miR-23a | atcacattgccagggatttcc (SEQ ID NO: 127) | ggaaatccctggcaatgtgatggaaatccctggcaatgtgat (SEQ ID NO: 128) |
| miR-23b | atcacattgccagggattacc (SEQ ID NO: 129) | ggtaatccctggcaatgtgatggtaatccctggcaatgtgat (SEQ ID NO: 130) |
| miR-24 | tggctcagttcagcaggaacag (SEQ ID NO: 131) | ctgttcctgctgaactgagccactgttcctgctgaactgagcca (SEQ ID NO: 132) |
| miR-25 | cattgcacttgtctcggtctga (SEQ ID NO: 133) | tcagaccgagacaagtgcaatgtcagaccgagacaagtgcaatg (SEQ ID NO: 134) |
| miR-26a | ttcaagtaatccaggataggc (SEQ ID NO: 135) | gcctatcctggattacttgaagcctatcctggattacttgaa (SEQ ID NO: 136) |
| miR-26b | ttcaagtaattcaggataggtt (SEQ ID NO: 137) | aacctatcctgaattacttgaaaacctatcctgaattacttgaa (SEQ ID NO: 138) |
| miR-27a | ttcacagtggctaagttccgc (SEQ ID NO: 139) | gcggaacttagccactgtgaagcggaacttagccactgtgaa (SEQ ID NO: 140) |
| miR-27b | ttcacagtggctaagttctgc (SEQ ID NO: 141) | gcagaacttagccactgtgaagcagaacttagccactgtgaa (SEQ ID NO: 142) |
| miR-28 | aaggagctcacagtctattgag (SEQ ID NO: 143) | ctcaatagactgtgagctccttctcaatagactgtgagctcctt (SEQ ID NO: 144) |
| miR-29a | tagcaccatctgaaatcggtt (SEQ ID NO: 145) | aaccgatttcagatggtgctaaaccgatttcagatggtgcta (SEQ ID NO: 146) |
| miR-29b | tagcaccatttgaaatcagtgtt (SEQ ID NO: 147) | aacactgatttcaaatggtgctaaacactgatttcaaatggtgcta (SEQ ID NO: 148) |
| miR-29c | tagcaccatttgaaatcggt (SEQ ID NO: 149) | accgatttcaaatggtgctaaccgatttcaaatggtgcta (SEQ ID NO: 150) |
| miR-30a-3p | ctttcagtcggatgtttgcagc (SEQ ID NO: 151) | gctgcaaacatccgactgaaaggctgcaaacatccgactgaaag (SEQ ID NO: 152) |
| miR-30a-5p | tgtaaacatcctcgactggaag (SEQ ID NO: 153) | cttccagtcgaggatgtttacacttccagtcgaggatgtttaca (SEQ ID NO: 154) |
| miR-30b | tgtaaacatcctacactcagct (SEQ ID NO: 155) | agctgagtgtaggatgtttacaagctgagtgtaggatgtttaca (SEQ ID NO: 156) |
| miR-30c | tgtaaacatcctacactctcagc (SEQ ID NO: 157) | gctgagagtgtaggatgtttacagctgagagtgtaggatgtttaca (SEQ ID NO: 158) |
| miR-30d | tgtaaacatccccgactggaag (SEQ ID NO: 159) | cttccagtcggggatgtttacacttccagtcggggatgtttaca (SEQ ID NO: 160) |
| miR-30e-3p | ctttcagtcggatgtttacagc (SEQ ID NO: 161) | gctgtaaacatccgactgaaaggctgtaaacatccgactgaaag (SEQ ID NO: 162) |
| miR-30e-5p | tgtaaacatccttgactgga (SEQ ID NO: 163) | tccagtcaaggatgtttacatccagtcaaggatgtttaca (SEQ ID NO: 164) |
| miR-31 | ggcaagatgctggcatagctg (SEQ ID NO: 165) | cagctatgccagcatcttgcccagctatgccagcatcttgcc (SEQ ID NO: 166) |
| miR-32 | tattgcacattactaagttgc (SEQ ID NO: 167) | gcaacttagtaatgtgcaatagcaacttagtaatgtgcaata (SEQ ID NO: 168) |
| miR-33 | gtgcattgtagttgcattg (SEQ ID NO: 169) | caatgcaactacaatgcaccaatgcaactacaatgcac (SEQ ID NO: 170) |
| miR-34a | tggcagtgtcttagctggttgtt (SEQ ID NO: 171) | aacaaccagctaagacactgccaaacaaccagctaagacactgcca (SEQ ID NO: 172) |
| miR-34b | taggcagtgtcattagctgattg (SEQ ID NO: 173) | caatcagctaatgacactgcctacaatcagctaatgacactgccta (SEQ ID NO: 174) |
| miR-34c | aggcagtgtagttagctgattgc (SEQ ID NO: 175) | gcaatcagctaactacactgcctgcaatcagctaactacactgcct (SEQ ID NO: 176) |
| miR-92 | tattgcacttgtcccggcctg (SEQ ID NO: 177) | caggccgggacaagtgcaatacaggccgggacaagtgcaata (SEQ ID NO: 178) |
| miR-93 | aaagtgctgttcgtgcaggtag (SEQ ID NO: 179) | ctacctgcacgaacagcactttctacctgcacgaacagcacttt (SEQ ID NO: 180) |
| miR-95 | ttcaacgggtatttattgagca (SEQ ID NO: 181) | tgctcaataaatacccgttgaatgctcaataaatacccgttgaa (SEQ ID NO: 182) |
| miR-96 | tttggcactagcacatttttgc (SEQ ID NO: 183) | gcaaaaatgtgctagtgccaaagcaaaaatgtgctagtgccaaa (SEQ ID NO: 184) |
| miR-98 | tgaggtagtaagttgtattgtt (SEQ ID NO: 185) | aacaatacaacttactacctcaaacaatacaacttactacctca (SEQ ID NO: 186) |
| miR-99a | aacccgtagatccgatcttgtg (SEQ ID NO: 187) | cacaagatcggatctacgggttcacaagatcggatctacgggtt (SEQ ID NO: 188) |
| miR-99b | cacccgtagaaccgaccttgcg (SEQ ID NO: 189) | cgcaaggtcggttctacgggtgcgcaaggtcggttctacgggtg (SEQ ID NO: 190) |
| miR-100 | aacccgtagatccgaacttgtg (SEQ ID NO: 191) | cacaagttcggatctacgggttcacaagttcggatctacgggtt (SEQ ID NO: 192) |
| miR-101 | tacagtactgtgataactgaag (SEQ ID NO: 193) | cttcagttatcacagtactgtacttcagttatcacagtactgta (SEQ ID NO: 194) |
| miR-103 | agcagcattgtacagggctatga (SEQ ID NO: 195) | tcatagccctgtacaatgctgcttcatagccctgtacaatgctgct (SEQ ID NO: 196) |
| miR-105 | tcaaatgctcagactcctgt (SEQ ID NO: 197) | acaggagtctgagcatttgaacaggagtctgagcatttga (SEQ ID NO: 198) |
| miR-106a | aaaagtgcttacagtgcaggtagc (SEQ ID NO: 199) | gctacctgcactgtaagcacttttgctacctgcactgtaagcactttt (SEQ ID NO: 200) |
| miR-106b | taaagtgctgacagtgcagat (SEQ ID NO: 201) | atctgcactgtcagcactttaatctgcactgtcagcacttta (SEQ ID NO: 202) |
| miR-107 | agcagcattgtacagggctatca (SEQ ID NO: 203) | tgatagccctgtacaatgctgcttgatagccctgtacaatgctgct (SEQ ID NO: 204) |
| miR-108 | ataaggatttttaggggcatt (SEQ ID NO: 205) | aatgcccctaaaaatccttataatgcccctaaaaatccttat (SEQ ID NO: 206) |
| miR-122a | tggagtgtgacaatggtgtttgt (SEQ ID NO: 207) | acaaacaccattgtcacactccaacaaacaccattgtcacactcca (SEQ ID NO: 208) |
| miR-124a | ttaaggcacgcggtgaatgcca (SEQ ID NO: 209) | tggcattcaccgcgtgccttaatggcattcaccgcgtgccttaa (SEQ ID NO: 210) |
| miR-125a | tccctgagaccctttaacctgtg (SEQ ID NO: 211) | cacaggttaaagggtctcagggacacaggttaaagggtctcaggga (SEQ ID NO: 212) |
| miR-125b | tccctgagaccctaacttgtga (SEQ ID NO: 213) | tcacaagttagggtctcagggatcacaagttagggtctcaggga (SEQ ID NO: 214) |
| miR-126 | tcgtaccgtgagtaataatgc (SEQ ID NO: 215) | gcattattactcacggtacgagcattattactcacggtacga (SEQ ID NO: 216) |
| miR-126* | cattattacttttggtacgcg (SEQ ID NO: 217) | cgcgtaccaaaagtaataatgcgcgtaccaaaagtaataatg (SEQ ID NO: 218) |
| miR-127 | tcggatccgtctgagcttggct (SEQ ID NO: 219) | agccaagctcagacggatccgaagccaagctcagacggatccga (SEQ ID NO: 220) |
| miR-128a | tcacagtgaaccggtctctttt (SEQ ID NO: 221) | aaaagagaccggttcactgtgaaaaagagaccggttcactgtga (SEQ ID NO: 222) |
| miR-128b | tcacagtgaaccggtctctttc (SEQ ID NO: 223) | gaaagagaccggttcactgtgagaaagagaccggttcactgtga (SEQ ID NO: 224) |
| miR-129 | ctttttgcggtctgggcttgc (SEQ ID NO: 225) | gcaagcccagaccgcaaaaaggcaagcccagaccgcaaaaag (SEQ ID NO: 226) |
| miR-130a | cagtgcaatgttaaaagggcat (SEQ ID NO: 227) | atgcccttttaacattgcactgatgcccttttaacattgcactg (SEQ ID NO: 228) |
| miR-130b | cagtgcaatgatgaaagggcat (SEQ ID NO: 229) | atgccctttcatcattgcactgatgccctttcatcattgcactg (SEQ ID NO: 230) |
| miR-132 | taacagtctacagccatggtcg (SEQ ID NO: 231) | cgaccatggctgtagactgttacgaccatggctgtagactgtta (SEQ ID NO: 232) |
| miR-133a | ttggtccccttcaaccagctgt (SEQ ID NO: 233) | acagctggttgaaggggaccaaacagctggttgaaggggaccaa (SEQ ID NO: 234) |
| miR-133b | ttggtccccttcaaccagcta (SEQ ID NO: 235) | tagctggttgaaggggaccaatagctggttgaaggggaccaa (SEQ ID NO: 236) |
| miR-134 | tgtgactggttgaccagaggg (SEQ ID NO: 237) | ccctctggtcaaccagtcacaccctctggtcaaccagtcaca (SEQ ID NO: 238) |
| miR-135a | tatggctttttattcctatgtga (SEQ ID NO: 239) | tcacataggaataaaaagccatatcacataggaataaaaagccata (SEQ ID NO: 240) |
| miR-135b | tatggcttttcattcctatgtg (SEQ ID NO: 241) | cacataggaatgaaaagccatacacataggaatgaaaagccata (SEQ ID NO: 242) |
| miR-136 | actccatttgttttgatgatgga (SEQ ID NO: 243) | tccatcatcaaaacaaatggagttccatcatcaaaacaaatggagt (SEQ ID NO: 244) |
| miR-137 | tattgcttaagaatacgcgtag (SEQ ID NO: 245) | ctacgcgtattcttaagcaatactacgcgtattcttaagcaata (SEQ ID NO: 246) |
| miR-138 | agctggtgttgtgaatc (SEQ ID NO: 247) | gattcacaacaccagctgattcacaacaccagct (SEQ ID NO: 248) |
| miR-139 | tctacagtgcacgtgtct (SEQ ID NO: 249) | agacacgtgcactgtagaagacacgtgcactgtaga (SEQ ID NO: 250) |
| miR-140 | agtggttttaccctatggtag (SEQ ID NO: 251) | ctaccatagggtaaaaccactctaccatagggtaaaaccact (SEQ ID NO: 252) |
| miR-141 | taacactgtctggtaaagatgg (SEQ ID NO: 253) | ccatctttaccagacagtgttaccatctttaccagacagtgtta (SEQ ID NO: 254) |
| miR-142-3p | tgtagtgtttcctactttatgga (SEQ ID NO: 255) | tccataaagtaggaaacactacatccataaagtaggaaacactaca (SEQ ID NO: 256) |
| miR-142-5p | cataaagtagaaagcactac (SEQ ID NO: 257) | gtagtgctttctactttatggtagtgctttctactttatg (SEQ ID NO: 258) |
| miR-143 | tgagatgaagcactgtagctca (SEQ ID NO: 259) | tgagctacagtgcttcatctcatgagctacagtgcttcatctca (SEQ ID NO: 260) |
| miR-144 | tacagtatagatgatgtactag (SEQ ID NO: 261) | ctagtacatcatctatactgtactagtacatcatctatactgta (SEQ ID NO: 262) |
| miR-145 | gtccagttttcccaggaatccctt (SEQ ID NO: 263) | aagggattcctgggaaaactggacaagggattcctgggaaaactggac (SEQ ID NO: 264) |
| miR-146 | tgagaactgaattccatgggtt (SEQ ID NO: 265) | aacccatggaattcagttctcaaacccatggaattcagttctca (SEQ ID NO: 266) |
| miR-147 | gtgtgtggaaatgcttctgc (SEQ ID NO: 267) | gcagaagcatttccacacacgcagaagcatttccacacac (SEQ ID NO: 268) |
| miR-148a | tcagtgcactacagaactttgt (SEQ ID NO: 269) | acaaagttctgtagtgcactgaacaaagttctgtagtgcactga (SEQ ID NO: 270) |
| miR-148b | tcagtgcatcacagaactttgt (SEQ ID NO: 271) | acaaagttctgtgatgcactgaacaaagttctgtgatgcactga (SEQ ID NO: 272) |
| miR-149 | tctggctccgtgtcttcactcc (SEQ ID NO: 273) | ggagtgaagacacggagccagaggagtgaagacacggagccaga (SEQ ID NO: 274) |
| miR-150 | tctcccaacccttgtaccagtg (SEQ ID NO: 275) | cactggtacaagggttgggagacactggtacaagggttgggaga (SEQ ID NO: 276) |
| miR-151 | actagactgaagctccttgagg (SEQ ID NO: 277) | cctcaaggagcttcagtctagtcctcaaggagcttcagtctagt (SEQ ID NO: 278) |
| miR-152 | tcagtgcatgacagaacttggg (SEQ ID NO: 279) | cccaagttctgtcatgcactgacccaagttctgtcatgcactga (SEQ ID NO: 280) |
| miR-153 | ttgcatagtcacaaaagtga (SEQ ID NO: 281) | tcacttttgtgactatgcaatcacttttgtgactatgcaa (SEQ ID NO: 282) |
| miR-154 | taggttatccgtgttgccttcg (SEQ ID NO: 283) | cgaaggcaacacggataacctacgaaggcaacacggataaccta (SEQ ID NO: 284) |
| miR-154* | aatcatacacggttgacctatt (SEQ ID NO: 285) | aataggtcaaccgtgtatgattaataggtcaaccgtgtatgatt (SEQ ID NO: 286) |
| miR-155 | ttaatgctaatcgtgatagggg (SEQ ID NO: 287) | cccctatcacgattagcattaacccctatcacgattagcattaa (SEQ ID NO: 288) |
| miR-181a | aacattcaacgctgtcggtgagt (SEQ ID NO: 289) | actcaccgacagcgttgaatgttactcaccgacagcgttgaatgtt (SEQ ID NO: 290) |
| miR-181b | aacattcattgctgtcggtggg (SEQ ID NO: 291) | cccaccgacagcaatgaatgttcccaccgacagcaatgaatgtt (SEQ ID NO: 292) |
| miR-181c | aacattcaacctgtcggtgagt (SEQ ID NO: 293) | actcaccgacaggttgaatgttactcaccgacaggttgaatgtt (SEQ ID NO: 294) |
| miR-182 | tttggcaatggtagaactcaca (SEQ ID NO: 295) | tgtgagttctaccattgccaaatgtgagttctaccattgccaaa (SEQ ID NO: 296) |
| miR-182* | tggttctagacttgccaacta (SEQ ID NO: 297) | tagttggcaagtctagaaccatagttggcaagtctagaacca (SEQ ID NO: 298) |
| miR-183 | tatggcactggtagaattcactg (SEQ ID NO: 299) | cagtgaattctaccagtgccatacagtgaattctaccagtgccata (SEQ ID NO: 300) |
| miR-184 | tggacggagaactgataagggt (SEQ ID NO: 301) | acccttatcagttctccgtccaacccttatcagttctccgtcca (SEQ ID NO: 302) |
| miR-185 | tggagagaaaggcagttc (SEQ ID NO: 303) | gaactgcctttctctccagaactgcctttctctcca (SEQ ID NO: 304) |
| miR-186 | caaagaattctccttttgggctt (SEQ ID NO: 305) | aagcccaaaaggagaattctttgaagcccaaaaggagaattctttg (SEQ ID NO: 306) |
| miR-187 | tcgtgtcttgtgttgcagccg (SEQ ID NO: 307) | cggctgcaacacaagacacgacggctgcaacacaagacacga (SEQ ID NO: 308) |
| miR-188 | catcccttgcatggtggagggt (SEQ ID NO: 309) | accctccaccatgcaagggatgaccctccaccatgcaagggatg (SEQ ID NO: 310) |
| miR-189 | gtgcctactgagctgatatcagt (SEQ ID NO: 311) | actgatatcagctcagtaggcacactgatatcagctcagtaggcac (SEQ ID NO: 312) |
| miR-190 | tgatatgtttgatatattaggt (SEQ ID NO: 313) | acctaatatatcaaacatatcaacctaatatatcaaacatatca (SEQ ID NO: 314) |
| miR-191 | caacggaatcccaaaagcagct (SEQ ID NO: 315) | agctgcttttgggattccgttgagctgcttttgggattccgttg (SEQ ID NO: 316) |
| miR-192 | ctgacctatgaattgacagcc (SEQ ID NO: 317) | ggctgtcaattcataggtcagggctgtcaattcataggtcag (SEQ ID NO: 318) |
| miR-193 | aactggcctacaaagtcccag (SEQ ID NO: 319) | ctgggactttgtaggccagttctgggactttgtaggccagtt (SEQ ID NO: 320) |
| miR-194 | tgtaacagcaactccatgtgga (SEQ ID NO: 321) | tccacatggagttgctgttacatccacatggagttgctgttaca (SEQ ID NO: 322) |
| miR-195 | tagcagcacagaaatattggc (SEQ ID NO: 323) | gccaatatttctgtgctgctagccaatatttctgtgctgcta (SEQ ID NO: 324) |
| miR-196a | taggtagtttcatgttgttgg (SEQ ID NO: 325) | ccaacaacatgaaactacctaccaacaacatgaaactaccta (SEQ ID NO: 326) |
| miR-196b | taggtagtttcctgttgttgg (SEQ ID NO: 327) | ccaacaacaggaaactacctaccaacaacaggaaactaccta (SEQ ID NO: 328) |
| miR-197 | ttcaccaccttctccacccagc (SEQ ID NO: 329) | gctgggtggagaaggtggtgaagctgggtggagaaggtggtgaa (SEQ ID NO: 330) |
| miR-198 | ggtccagaggggagatagg (SEQ ID NO: 331) | cctatctcccctctggacccctatctcccctctggacc (SEQ ID NO: 332) |
| miR-199a | cccagtgttcagactacctgttc (SEQ ID NO: 333) | gaacaggtagtctgaacactggggaacaggtagtctgaacactggg (SEQ ID NO: 334) |
| miR-199a* | tacagtagtctgcacattggtt (SEQ ID NO: 335) | aaccaatgtgcagactactgtaaaccaatgtgcagactactgta (SEQ ID NO: 336) |
| miR-199b | cccagtgtttagactatctgttc (SEQ ID NO: 337) | gaacagatagtctaaacactggggaacagatagtctaaacactggg (SEQ ID NO: 338) |
| miR-200a | taacactgtctggtaacgatgt (SEQ ID NO: 339) | acatcgttaccagacagtgttaacatcgttaccagacagtgtta (SEQ ID NO: 340) |
| miR-200b | taatactgcctggtaatgatgac (SEQ ID NO: 341) | gtcatcattaccaggcagtattagtcatcattaccaggcagtatta (SEQ ID NO: 342) |
| miR-200c | taatactgccgggtaatgatgg (SEQ ID NO: 343) | ccatcattacccggcagtattaccatcattacccggcagtatta (SEQ ID NO: 344) |
| miR-203 | gtgaaatgtttaggaccactag (SEQ ID NO: 345) | ctagtggtcctaaacatttcacctagtggtcctaaacatttcac (SEQ ID NO: 346) |
| miR-204 | ttccctttgtcatcctatgcct (SEQ ID NO: 347) | aggcataggatgacaaagggaaaggcataggatgacaaagggaa (SEQ ID NO: 348) |
| miR-205 | tccttcattccaccggagtctg (SEQ ID NO: 349) | cagactccggtggaatgaaggacagactccggtggaatgaagga (SEQ ID NO: 350) |
| miR-206 | tggaatgtaaggaagtgtgtgg (SEQ ID NO: 351) | ccacacacttccttacattccaccacacacttccttacattcca (SEQ ID NO: 352) |
| miR-208 | ataagacgagcaaaaagcttgt (SEQ ID NO: 353) | acaagctttttgctcgtcttatacaagctttttgctcgtcttat (SEQ ID NO: 354) |
| miR-210 | ctgtgcgtgtgacagcggctga (SEQ ID NO: 355) | tcagccgctgtcacacgcacagtcagccgctgtcacacgcacag (SEQ ID NO: 356) |
| miR-211 | ttccctttgtcatccttcgcct (SEQ ID NO: 357) | aggcgaaggatgacaaagggaaaggcgaaggatgacaaagggaa (SEQ ID NO: 358) |
| miR-212 | taacagtctccagtcacggcc (SEQ ID NO: 359) | ggccgtgactggagactgttaggccgtgactggagactgtta (SEQ ID NO: 360) |
| miR-213 | accatcgaccgttgattgtacc (SEQ ID NO: 361) | ggtacaatcaacggtcgatggtggtacaatcaacggtcgatggt (SEQ ID NO: 362) |
| miR-214 | acagcaggcacagacaggcag (SEQ ID NO: 363) | ctgcctgtctgtgcctgctgtctgcctgtctgtgcctgctgt (SEQ ID NO: 364) |
| miR-215 | atgacctatgaattgacagac (SEQ ID NO: 365) | gtctgtcaattcataggtcatgtctgtcaattcataggtcat (SEQ ID NO: 366) |
| miR-216 | taatctcagctggcaactgtg (SEQ ID NO: 367) | cacagttgccagctgagattacacagttgccagctgagatta (SEQ ID NO: 368) |
| miR-217 | tactgcatcaggaactgattggat (SEQ ID NO: 369) | atccaatcagttcctgatgcagtaatccaatcagttcctgatgcagta (SEQ ID NO: 370) |
| miR-218 | ttgtgcttgatctaaccatgt (SEQ ID NO: 371) | acatggttagatcaagcacaaacatggttagatcaagcacaa (SEQ ID NO: 372) |
| miR-219 | tgattgtccaaacgcaattct (SEQ ID NO: 373) | agaattgcgtttggacaatcaagaattgcgtttggacaatca (SEQ ID NO: 374) |
| miR-220 | ccacaccgtatctgacacttt (SEQ ID NO: 375) | aaagtgtcagatacggtgtggaaagtgtcagatacggtgtgg (SEQ ID NO: 376) |
| miR-221 | agctacattgtctgctgggtttc (SEQ ID NO: 377) | gaaacccagcagacaatgtagctgaaacccagcagacaatgtagct (SEQ ID NO: 378) |
| miR-222 | agctacatctggctactgggtctc (SEQ ID NO: 379) | gagacccagtagccagatgtagctgagacccagtagccagatgtagct (SEQ ID NO: 380) |
| miR-223 | tgtcagtttgtcaaatacccc (SEQ ID NO: 381) | ggggtatttgacaaactgacaggggtatttgacaaactgaca (SEQ ID NO: 382) |
| miR-224 | caagtcactagtggttccgttta (SEQ ID NO: 383) | taaacggaaccactagtgacttgtaaacggaaccactagtgacttg (SEQ ID NO: 384) |
| miR-296 | agggccccccctcaatcctgt (SEQ ID NO: 385) | acaggattgagggggggccctacaggattgagggggggccct (SEQ ID NO: 386) |
| miR-299 | tggtttaccgtcccacatacat (SEQ ID NO: 387) | atgtatgtgggacggtaaaccaatgtatgtgggacggtaaacca (SEQ ID NO: 388) |
| miR-301 | cagtgcaatagtattgtcaaagc (SEQ ID NO: 389) | gctttgacaatactattgcactggctttgacaatactattgcactg (SEQ ID NO: 390) |
| miR-302a | taagtgcttccatgttttggtga (SEQ ID NO: 391) | tcaccaaaacatggaagcacttatcaccaaaacatggaagcactta (SEQ ID NO: 392) |
| miR-302a* | taaacgtggatgtacttgcttt (SEQ ID NO: 393) | aaagcaagtacatccacgtttaaaagcaagtacatccacgttta (SEQ ID NO: 394) |
| miR-302b | taagtgcttccatgttttagtag (SEQ ID NO: 395) | ctactaaaacatggaagcacttactactaaaacatggaagcactta (SEQ ID NO: 396) |
| miR-302b* | actttaacatggaagtgctttct (SEQ ID NO: 397) | agaaagcacttccatgttaaagtagaaagcacttccatgttaaagt (SEQ ID NO: 398) |
| miR-302c | taagtgcttccatgtttcagtgg (SEQ ID NO: 399) | ccactgaaacatggaagcacttaccactgaaacatggaagcactta (SEQ ID NO: 400) |
| miR-302c* | tttaacatgggggtacctgctg (SEQ ID NO: 401) | cagcaggtacccccatgttaaacagcaggtacccccatgttaaa (SEQ ID NO: 402) |
| miR-302d | taagtgcttccatgtttgagtgt (SEQ ID NO: 403) | acactcaaacatggaagcacttaacactcaaacatggaagcactta (SEQ ID NO: 404) |
| miR-320 | aaaagctgggttgagagggcgaa (SEQ ID NO: 405) | ttcgccctctcaacccagcttttttcgccctctcaacccagctttt (SEQ ID NO: 406) |
| miR-323 | gcacattacacggtcgacctct (SEQ ID NO: 407) | agaggtcgaccgtgtaatgtgcagaggtcgaccgtgtaatgtgc (SEQ ID NO: 408) |
| miR-324-3p | ccactgccccaggtgctgctgg (SEQ ID NO: 409) | ccagcagcacctggggcagtggccagcagcacctggggcagtgg (SEQ ID NO: 410) |
| miR-324-5p | cgcatcccctagggcattggtgt (SEQ ID NO: 411) | acaccaatgccctaggggatgcgacaccaatgccctaggggatgcg (SEQ ID NO: 412) |
| miR-325 | cctagtaggtgtccagtaagtgt (SEQ ID NO: 413) | acacttactggacacctactaggacacttactggacacctactagg (SEQ ID NO: 414) |
| miR-326 | cctctgggcccttcctccag (SEQ ID NO: 415) | ctggaggaagggcccagaggctggaggaagggcccagagg (SEQ ID NO: 416) |
| miR-328 | ctggccctctctgcccttccgt (SEQ ID NO: 417) | acggaagggcagagagggccagacggaagggcagagagggccag (SEQ ID NO: 418) |
| miR-330 | gcaaagcacacggcctgcagaga (SEQ ID NO: 419) | tctctgcaggccgtgtgctttgctctctgcaggccgtgtgctttgc (SEQ ID NO: 420) |
| miR-331 | gcccctgggcctatcctagaa (SEQ ID NO: 421) | ttctaggataggcccaggggcttctaggataggcccaggggc (SEQ ID NO: 422) |
| miR-335 | tcaagagcaataacgaaaaatgt (SEQ ID NO: 423) | acatttttcgttattgctcttgaacatttttcgttattgctcttga (SEQ ID NO: 424) |
| miR-337 | tccagctcctatatgatgccttt (SEQ ID NO: 425) | aaaggcatcatataggagctggaaaaggcatcatataggagctgga (SEQ ID NO: 426) |
| miR-338 | tccagcatcagtgattttgttga (SEQ ID NO: 427) | tcaacaaaatcactgatgctggatcaacaaaatcactgatgctgga (SEQ ID NO: 428) |
| miR-339 | tccctgtcctccaggagctca (SEQ ID NO: 429) | tgagctcctggaggacagggatgagctcctggaggacaggga (SEQ ID NO: 430) |
| miR-340 | tccgtctcagttactttatagcc (SEQ ID NO: 431) | ggctataaagtaactgagacggaggctataaagtaactgagacgga (SEQ ID NO: 432) |
| miR-342 | tctcacacagaaatcgcacccgtc (SEQ ID NO: 433) | gacgggtgcgatttctgtgtgagagacgggtgcgatttctgtgtgaga (SEQ ID NO: 434) |
| miR-345 | tgctgactcctagtccagggc (SEQ ID NO: 435) | gccctggactaggagtcagcagccctggactaggagtcagca (SEQ ID NO: 436) |
| miR-346 | tgtctgcccgcatgcctgcctct (SEQ ID NO: 437) | agaggcaggcatgcgggcagacaagaggcaggcatgcgggcagaca (SEQ ID NO: 438) |
| miR-361 | ttatcagaatctccaggggtac (SEQ ID NO: 439) | gtacccctggagattctgataagtacccctggagattctgataa (SEQ ID NO: 440) |
| miR-367 | aattgcactttagcaatggtga (SEQ ID NO: 441) | tcaccattgctaaagtgcaatttcaccattgctaaagtgcaatt (SEQ ID NO: 442) |
| miR-368 | acatagaggaaattccacgttt (SEQ ID NO: 443) | aaacgtggaatttcctctatgtaaacgtggaatttcctctatgt (SEQ ID NO: 444) |
| miR-369 | aataatacatggttgatcttt (SEQ ID NO: 445) | aaagatcaaccatgtattattaaagatcaaccatgtattatt (SEQ ID NO: 446) |
| miR-370 | gcctgctggggtggaacctgg (SEQ ID NO: 447) | ccaggttccaccccagcaggcccaggttccaccccagcaggc (SEQ ID NO: 448) |
| miR-371 | gtgccgccatcttttgagtgt (SEQ ID NO: 449) | acactcaaaagatggcggcacacactcaaaagatggcggcac (SEQ ID NO: 450) |
| miR-372 | aaagtgctgcgacatttgagcgt (SEQ ID NO: 451) | acgctcaaatgtcgcagcactttacgctcaaatgtcgcagcacttt (SEQ ID NO: 452) |
| miR-373 | gaagtgcttcgattttggggtgt (SEQ ID NO: 453) | acaccccaaaatcgaagcacttcacaccccaaaatcgaagcacttc (SEQ ID NO: 454) |
| miR-373* | actcaaaatgggggcgctttcc (SEQ ID NO: 455) | ggaaagcgcccccattttgagtggaaagcgcccccattttgagt (SEQ ID NO: 456) |
| miR-374 | ttataatacaacctgataagtg (SEQ ID NO: 457) | cacttatcaggttgtattataacacttatcaggttgtattataa (SEQ ID NO: 458) |
| miR-375 | tttgttcgttcggctcgcgtga (SEQ ID NO: 459) | tcacgcgagccgaacgaacaaatcacgcgagccgaacgaacaaa (SEQ ID NO: 460) |
| miR-376a | atcatagaggaaaatccacgt (SEQ ID NO: 461) | acgtggattttcctctatgatacgtggattttcctctatgat (SEQ ID NO: 462) |
| miR-377 | atcacacaaaggcaacttttgt (SEQ ID NO: 463) | acaaaagttgcctttgtgtgatacaaaagttgcctttgtgtgat (SEQ ID NO: 464) |
| miR-378 | ctcctgactccaggtcctgtgt (SEQ ID NO: 465) | acacaggacctggagtcaggagacacaggacctggagtcaggag (SEQ ID NO: 466) |
| miR-379 | tggtagactatggaacgta (SEQ ID NO: 467) | tacgttccatagtctaccatacgttccatagtctacca (SEQ ID NO: 468) |
| miR-380-3p | tatgtaatatggtccacatctt (SEQ ID NO: 469) | aagatgtggaccatattacataaagatgtggaccatattacata (SEQ ID NO: 470) |
| miR-380-5p | tggttgaccatagaacatgcgc (SEQ ID NO: 471) | gcgcatgttctatggtcaaccagcgcatgttctatggtcaacca (SEQ ID NO: 472) |
| miR-381 | tatacaagggcaagctctctgt (SEQ ID NO: 473) | acagagagcttgcccttgtataacagagagcttgcccttgtata (SEQ ID NO: 474) |
| miR-382 | gaagttgttcgtggtggattcg (SEQ ID NO: 475) | cgaatccaccacgaacaacttccgaatccaccacgaacaacttc (SEQ ID NO: 476) |
| miR-383 | agatcagaaggtgattgtggct (SEQ ID NO: 477) | agccacaatcaccttctgatctagccacaatcaccttctgatct (SEQ ID NO: 478) |
| miR-384 | attcctagaaattgttcata (SEQ ID NO: 479) | tatgaacaatttctaggaattatgaacaatttctaggaat (SEQ ID NO: 480) |
| miR-422a | ctggacttagggtcagaaggcc (SEQ ID NO: 481) | ggccttctgaccctaagtccagggccttctgaccctaagtccag (SEQ ID NO: 482) |
| miR-422b | ctggacttggagtcagaaggcc (SEQ ID NO: 483) | ggccttctgactccaagtccagggccttctgactccaagtccag (SEQ ID NO: 484) |
| miR-423 | agctcggtctgaggcccctcag (SEQ ID NO: 485) | ctgaggggcctcagaccgagctctgaggggcctcagaccgagct (SEQ ID NO: 486) |
| miR-424 | cagcagcaattcatgttttgaa (SEQ ID NO: 487) | ttcaaaacatgaattgctgctgttcaaaacatgaattgctgctg (SEQ ID NO: 488) |
| miR-425 | atcgggaatgtcgtgtccgcc (SEQ ID NO: 489) | ggcggacacgacattcccgatggcggacacgacattcccgat (SEQ ID NO: 490) |
| D.melanog. miR-1 | tggaatgtaaagaagtatggag (SEQ ID NO: 491) | ctccatacttctttacattccactccatacttctttacattcca (SEQ ID NO: 492) |
| D.melanog. miR-2a | tatcacagccagctttgatgagc (SEQ ID NO: 493) | gctcatcaaagctggctgtgatagctcatcaaagctggctgtgata (SEQ ID NO: 494) |
| D.melanog. miR-3 | tcactgggcaaagtgtgtctca (SEQ ID NO: 495) | tgagacacactttgcccagtgatgagacacactttgcccagtga (SEQ ID NO: 496) |
| D.melanog. miR-4 | ataaagctagacaaccattga (SEQ ID NO: 497) | tcaatggttgtctagctttattcaatggttgtctagctttat (SEQ ID NO: 498) |
| D.melanog. miR-5 | aaaggaacgatcgttgtgatatg (SEQ ID NO: 499) | catatcacaacgatcgttcctttcatatcacaacgatcgttccttt (SEQ ID NO: 500) |
| D.melanog. miR-6 | tatcacagtggctgttcttttt (SEQ ID NO: 501) | aaaaagaacagccactgtgataaaaaagaacagccactgtgata (SEQ ID NO: 502) |
| D.melanog. bantarr | tgagatcattttgaaagctgatt (SEQ ID NO: 503) | aatcagctttcaaaatgatctcaaatcagctttcaaaatgatctca (SEQ ID NO: 504) |

*miRNAs numbered identically but distinguished by an asterisk are derived from different arms of the same precursor RNA.

TABLE 2
Expression values of all tested miRNAs in NPC Tumor and Normal tissues
Normal and Tumor medians were calculated from quantile normalized miRNA expression levels

| | Normal | Tumor | Fold difference | Wilcoxon** | Wilcoxon | t-test | t-test (log) |
|---|---|---|---|---|---|---|---|
| miRNA | median | median | (Tumor/Normal) | p-value | q-value | q-value | q-value |
| let-7a | 39035 | 44514 | 1.14 | 0.359 | 0.409 | 0.228 | 0.465 |
| let-7b | 55015 | 49450 | 0.90 | 0.052 | 0.103 | 0.003 | 0.01 |
| let-7c | 49450 | 49450 | 1.00 | 0.865 | 0.706 | 0.161 | 0.214 |
| let-7d | 21503 | 25933 | 1.21 | 0.273 | 0.338 | 0.216 | 0.392 |
| let-7e | 20493 | 34468 | 1.68 | 0.013 | 0.054 | 0.006 | 0.141 |
| let-7f | 16149 | 18520 | 1.15 | 0.475 | 0.499 | 0.142 | 0.355 |
| let-7g | 8766 | 6098 | 0.70 | 0.370 | 0.416 | 0.199 | 0.372 |
| let-7i | 5400 | 8101 | 1.50 | 0.073 | 0.134 | 0.199 | 0.174 |
| miR-1 | 83 | 98 | 1.17 | 0.281 | 0.341 | 0.01 | 0.214 |
| miR-7 | 124 | 46 | 0.37 | 0.197 | 0.276 | 0.238 | 0.139 |
| miR-9 | 4 | 6 | 1.43 | 0.867 | 0.706 | 0.198 | 0.439 |
| miR-9* | 121 | 112 | 0.92 | 0.554 | 0.557 | 0.14 | 0.218 |
| miR-10a | 37 | 60 | 1.61 | 0.125 | 0.198 | 0.098 | 0.153 |
| miR-10b | 57 | 65 | 1.15 | 0.693 | 0.631 | 0.161 | 0.291 |
| miR-15a | 747 | 3252 | 4.36 | 0.003 | 0.024 | 0.004 | 0.007 |
| miR-15b | 12095 | 29506 | 2.44 | 0.011 | 0.05 | 0.022 | 0.019 |
| miR-16 | 10055 | 21781 | 2.17 | 0.001 | 0.01 | 0 | 0 |
| miR-17-3p | 2643 | 3252 | 1.23 | 0.843 | 0.706 | 0.139 | 0.417 |
| miR-17-5p | 720 | 1230 | 1.71 | 0.192 | 0.274 | 0.111 | 0.187 |
| miR-18 | 136 | 885 | 6.53 | 0.044 | 0.094 | 0.044 | 0.043 |
| miR-19a | 202 | 363 | 1.80 | 0.230 | 0.302 | 0.039 | 0.247 |
| miR-19b | 1901 | 4861 | 2.56 | 0.029 | 0.072 | 0.153 | 0.085 |
| miR-20 | 1227 | 1292 | 1.05 | 0.466 | 0.493 | 0.216 | 0.32 |
| miR-21 | 9892 | 8101 | 0.82 | 0.867 | 0.706 | 0.199 | 0.417 |
| miR-22 | 1377 | 2715 | 1.97 | 0.089 | 0.151 | 0.005 | 0.25 |
| miR-23a | 4355 | 4024 | 0.92 | 0.716 | 0.637 | 0.208 | 0.405 |
| miR-23b | 7581 | 7862 | 1.04 | 0.903 | 0.714 | 0.199 | 0.392 |
| miR-24 | 19915 | 15841 | 0.80 | 0.421 | 0.457 | 0.142 | 0.391 |
| miR-25 | 12574 | 19659 | 1.56 | 0.028 | 0.072 | 0.01 | 0.092 |
| miR-26a | 9412 | 15841 | 1.68 | 0.026 | 0.068 | 0.005 | 0.046 |
| miR-26b | 162 | 1046 | 6.47 | 0.019 | 0.06 | 0.001 | 0.023 |
| miR-27a | 545 | 1046 | 1.92 | 0.019 | 0.06 | 0.002 | 0.036 |
| miR-27b | 607 | 1395 | 2.30 | 0.081 | 0.143 | 0.002 | 0.115 |
| miR-28 | 64 | 65 | 1.02 | 0.903 | 0.714 | 0.198 | 0.274 |
| miR-29a | 46930 | 34468 | 0.73 | 0.009 | 0.044 | 0 | 0 |
| miR-29b | 8061 | 2085 | 0.26 | 0.048 | 0.102 | 0.112 | 0.021 |
| miR-29c | 32320 | 6567 | 0.20 | 0.002 | 0.018 | 0 | 0 |
| miR-30a-3p | 1546 | 1011 | 0.65 | 0.808 | 0.685 | 0.249 | 0.314 |
| miR-30a-5p | 48 | 460 | 9.61 | 0.108 | 0.175 | 0.22 | 0.155 |
| miR-30b | 2178 | 2897 | 1.33 | 0.339 | 0.394 | 0.079 | 0.25 |
| miR-30c | 7841 | 7328 | 0.93 | 0.670 | 0.62 | 0.124 | 0.258 |
| miR-30d | 3107 | 8736 | 2.81 | 0.004 | 0.03 | 0 | 0.012 |
| miR-30e-3p | 1069 | 1230 | 1.15 | 0.176 | 0.261 | 0.035 | 0.155 |
| miR-30e-5p | 639 | 1092 | 1.71 | 0.274 | 0.338 | 0.218 | 0.405 |
| miR-31 | 6182 | 4702 | 0.76 | 0.595 | 0.577 | 0.25 | 0.274 |
| miR-32 | 380 | 142 | 0.37 | 0.125 | 0.198 | 0.076 | 0.189 |
| miR-33 | 10 | 6 | 0.58 | 0.915 | 0.719 | 0.183 | 0.411 |
| miR-34a | 23409 | 20376 | 0.87 | 0.438 | 0.47 | 0.175 | 0.206 |
| miR-34b | 28879 | 3252 | 0.11 | 0.000 | 0.002 | 0 | 0 |
| miR-34c | 25243 | 1461 | 0.06 | 0.001 | 0.01 | 0 | 0.004 |
| miR-92 | 16784 | 10513 | 0.63 | 0.015 | 0.054 | 0.009 | 0.007 |
| miR-93 | 13316 | 6567 | 0.49 | 0.316 | 0.381 | 0.175 | 0.404 |
| miR-95 | 7 | 7 | 0.95 | 0.940 | 0.725 | 0.216 | 0.479 |
| miR-96 | 2592 | 743 | 0.29 | 0.019 | 0.06 | 0.083 | 0.031 |
| miR-98 | 484 | 970 | 2.01 | 0.023 | 0.064 | 0.006 | 0.033 |
| miR-99a | 102 | 448 | 4.40 | 0.015 | 0.054 | 0.003 | 0.037 |
| miR-99b | 6230 | 7862 | 1.26 | 0.274 | 0.338 | 0.079 | 0.347 |
| miR-100 | 1121 | 1230 | 1.10 | 0.891 | 0.714 | 0.191 | 0.392 |
| miR-101 | 221 | 181 | 0.82 | 0.219 | 0.294 | 0.25 | 0.11 |
| miR-103 | 21976 | 39035 | 1.78 | 0.015 | 0.054 | 0.005 | 0.021 |
| miR-105 | 121 | 145 | 1.20 | 0.988 | 0.735 | 0.173 | 0.409 |
| miR-106a | 225 | 599 | 2.66 | 0.008 | 0.041 | 0.01 | 0.021 |
| miR-106b | 17104 | 11404 | 0.67 | 0.015 | 0.054 | 0.013 | 0.018 |
| miR-107 | 19052 | 21226 | 1.11 | 0.504 | 0.523 | 0.28 | 0.396 |
| miR-108 | 19 | 21 | 1.08 | 0.855 | 0.706 | 0.259 | 0.479 |
| miR-122a | 95 | 65 | 0.69 | 0.595 | 0.577 | 0.198 | 0.456 |
| miR-124a | 247 | 202 | 0.82 | 0.808 | 0.685 | 0.222 | 0.417 |
| miR-125a | 567 | 970 | 1.71 | 0.331 | 0.391 | 0.104 | 0.392 |
| miR-125b | 5118 | 12786 | 2.50 | 0.022 | 0.064 | 0.006 | 0.122 |
| miR-126 | 19477 | 10963 | 0.56 | 0.006 | 0.037 | 0.005 | 0.003 |
| miR-126* | 2050 | 1515 | 0.74 | 0.192 | 0.274 | 0.109 | 0.14 |
| miR-127 | 21078 | 10513 | 0.50 | 0.000 | 0.01 | 0 | 0 |
| miR-128a | 6964 | 3005 | 0.43 | 0.015 | 0.054 | 0.021 | 0.016 |
| miR-128b | 686 | 686 | 1.00 | 0.927 | 0.719 | 0.256 | 0.392 |
| miR-129 | 398 | 419 | 1.05 | 0.574 | 0.57 | 0.174 | 0.439 |
| miR-130a | 645 | 2897 | 4.49 | 0.078 | 0.14 | 0.002 | 0.076 |
| miR-130b | 4363 | 13891 | 3.18 | 0.001 | 0.016 | 0 | 0.006 |
| miR-132 | 238 | 145 | 0.61 | 0.192 | 0.274 | 0.142 | 0.333 |
| miR-133a | 2179 | 503 | 0.23 | 0.009 | 0.044 | 0.01 | 0.016 |
| miR-133b | 29506 | 20376 | 0.69 | 0.001 | 0.01 | 0 | 0 |
| miR-134 | 2645 | 3865 | 1.46 | 0.378 | 0.419 | 0.199 | 0.404 |
| miR-135a | 49 | 47 | 0.97 | 0.976 | 0.729 | 0.261 | 0.489 |
| miR-135b | 13 | 12 | 0.91 | 0.976 | 0.729 | 0.199 | 0.483 |
| miR-136 | 22 | 40 | 1.77 | 0.037 | 0.085 | 0.01 | 0.091 |
| miR-137 | 19 | 26 | 1.37 | 0.387 | 0.423 | 0.242 | 0.34 |
| miR-138 | 114 | 98 | 0.86 | 0.485 | 0.506 | 0.216 | 0.392 |
| miR-139 | 30 | 50 | 1.65 | 0.976 | 0.729 | 0.093 | 0.421 |
| miR-140 | 19 | 35 | 1.82 | 0.514 | 0.529 | 0.157 | 0.401 |
| miR-141 | 6956 | 8414 | 1.21 | 0.339 | 0.394 | 0.077 | 0.479 |
| miR-142-3p | 290 | 181 | 0.62 | 0.704 | 0.634 | 0.241 | 0.392 |
| miR-142-5p | 592 | 297 | 0.50 | 0.078 | 0.14 | 0.086 | 0.094 |
| miR-143 | 2392 | 7119 | 2.98 | 0.019 | 0.06 | 0.002 | 0.034 |
| miR-144 | 434 | 632 | 1.46 | 0.524 | 0.533 | 0.223 | 0.418 |
| miR-145 | 187 | 547 | 2.92 | 0.019 | 0.06 | 0.001 | 0.021 |
| miR-146 | 18520 | 12786 | 0.69 | 0.050 | 0.103 | 0.062 | 0.094 |
| miR-147 | 3944 | 1183 | 0.30 | 0.003 | 0.023 | 0.005 | 0 |
| miR-148a | 5635 | 3117 | 0.55 | 0.043 | 0.094 | 0.058 | 0.024 |
| miR-148b | 591 | 686 | 1.16 | 0.844 | 0.706 | 0.119 | 0.479 |
| miR-149 | 20801 | 19659 | 0.95 | 0.927 | 0.719 | 0.257 | 0.391 |
| miR-150 | 11649 | 17727 | 1.52 | 0.248 | 0.321 | 0.07 | 0.274 |
| miR-151 | 60 | 3598 | 60.25 | 0.001 | 0.01 | 0 | 0 |
| miR-152 | 3045 | 4355 | 1.43 | 0.207 | 0.286 | 0.035 | 0.076 |
| miR-153 | 252 | 400 | 1.59 | 0.387 | 0.423 | 0.049 | 0.392 |
| miR-154 | 310 | 410 | 1.33 | 0.346 | 0.4 | 0.185 | 0.25 |
| miR-154* | 577 | 95 | 0.16 | 0.012 | 0.05 | 0.087 | 0 |
| miR-155 | 27614 | 39035 | 1.41 | 0.019 | 0.06 | 0.042 | 0.085 |
| miR-181a | 7327 | 25933 | 3.54 | 0.001 | 0.018 | 0 | 0.066 |
| miR-181b | 11183 | 15249 | 1.36 | 0.050 | 0.103 | 0.029 | 0.078 |
| miR-181c | 40 | 145 | 3.64 | 0.036 | 0.084 | 0.004 | 0.086 |
| miR-182 | 2090 | 8736 | 4.18 | 0.010 | 0.047 | 0.004 | 0.051 |
| miR-182* | 401 | 567 | 1.41 | 0.255 | 0.327 | 0.278 | 0.252 |
| miR-183 | 575 | 1183 | 2.06 | 0.141 | 0.216 | 0.049 | 0.139 |
| miR-184 | 652 | 686 | 1.05 | 0.649 | 0.607 | 0.036 | 0.285 |
| miR-185 | 3549 | 4702 | 1.33 | 0.114 | 0.184 | 0.025 | 0.091 |
| miR-186 | 108 | 186 | 1.72 | 0.192 | 0.274 | 0.127 | 0.276 |
| miR-187 | 188 | 142 | 0.76 | 0.682 | 0.627 | 0.257 | 0.333 |
| miR-188 | 170 | 1092 | 6.42 | 0.027 | 0.07 | 0.142 | 0.043 |
| miR-189 | 20 | 50 | 2.54 | 0.054 | 0.105 | 0.256 | 0.128 |
| miR-190 | 8 | 16 | 1.96 | 0.750 | 0.657 | 0.123 | 0.392 |
| miR-191 | 8927 | 13344 | 1.49 | 0.016 | 0.055 | 0.006 | 0.133 |
| miR-192 | 71 | 1573 | 22.02 | 0.000 | 0.01 | 0.004 | 0 |
| miR-193 | 440 | 351 | 0.80 | 0.036 | 0.084 | 0.078 | 0.038 |
| miR-194 | 1116 | 2280 | 2.04 | 0.036 | 0.084 | 0.03 | 0.036 |
| miR-195 | 7224 | 5543 | 0.77 | 0.157 | 0.237 | 0.119 | 0.128 |
| miR-196a | 93 | 58 | 0.62 | 0.083 | 0.145 | 0.125 | 0.066 |
| miR-196b | 66 | 166 | 2.51 | 0.036 | 0.084 | 0.03 | 0.046 |
| miR-197 | 9674 | 5826 | 0.60 | 0.056 | 0.108 | 0.062 | 0.036 |
| miR-198 | 284 | 50 | 0.17 | 0.038 | 0.085 | 0.044 | 0.156 |
| miR-199a | 108 | 202 | 1.87 | 0.879 | 0.709 | 0.216 | 0.479 |
| miR-199a* | 869 | 2897 | 3.33 | 0.029 | 0.072 | 0.002 | 0.072 |
| miR-199b | 36 | 60 | 1.64 | 0.750 | 0.657 | 0.216 | 0.465 |
| miR-200a | 6230 | 6567 | 1.05 | 0.808 | 0.685 | 0.181 | 0.392 |
| miR-200b | 17812 | 13891 | 0.78 | 0.066 | 0.124 | 0.035 | 0.031 |
| miR-200c | 44514 | 44514 | 1.00 | 0.645 | 0.607 | 0.066 | 0.091 |
| miR-203 | 545 | 82 | 0.15 | 0.084 | 0.145 | 0.076 | 0.267 |
| miR-204 | 91 | 87 | 0.96 | 0.727 | 0.643 | 0.256 | 0.418 |
| miR-205 | 928 | 917 | 0.99 | 0.704 | 0.634 | 0.201 | 0.409 |
| miR-206 | 543 | 95 | 0.17 | 0.000 | 0.01 | 0.017 | 0 |
| miR-208 | 230 | 121 | 0.53 | 0.058 | 0.111 | 0.055 | 0.11 |
| miR-210 | 13338 | 13344 | 1.00 | 0.976 | 0.729 | 0.218 | 0.456 |
| miR-211 | 1488 | 479 | 0.32 | 0.002 | 0.018 | 0.008 | 0 |
| miR-212 | 4363 | 885 | 0.20 | 0.000 | 0.01 | 0.002 | 0 |
| miR-213 | 715 | 1011 | 1.42 | 0.133 | 0.206 | 0.01 | 0.066 |
| miR-214 | 32522 | 28147 | 0.87 | 0.224 | 0.297 | 0.104 | 0.122 |
| miR-215 | 1220 | 1515 | 1.24 | 1.000 | 0.74 | 0.218 | 0.439 |
| miR-216 | 6843 | 940 | 0.14 | 0.002 | 0.022 | 0.008 | 0 |
| miR-217 | 4212 | 351 | 0.08 | 0.000 | 0.01 | 0.001 | 0.002 |
| miR-218 | 18 | 40 | 2.19 | 0.129 | 0.201 | 0.064 | 0.139 |
| miR-219 | 131 | 130 | 0.99 | 0.964 | 0.729 | 0.218 | 0.392 |
| miR-220 | 2935 | 917 | 0.31 | 0.014 | 0.054 | 0.032 | 0.026 |
| miR-221 | 8736 | 10513 | 1.20 | 0.098 | 0.161 | 0.025 | 0.139 |
| miR-222 | 19433 | 20376 | 1.05 | 0.261 | 0.332 | 0.041 | 0.265 |
| miR-223 | 3419 | 2504 | 0.73 | 0.020 | 0.061 | 0.036 | 0.032 |
| miR-224 | 255 | 1046 | 4.10 | 0.008 | 0.041 | 0.005 | 0.036 |
| miR-296 | 7862 | 7581 | 0.96 | 0.867 | 0.706 | 0.233 | 0.456 |
| miR-299 | 221 | 65 | 0.30 | 0.370 | 0.416 | 0.238 | 0.188 |
| miR-301 | 54 | 98 | 1.81 | 0.197 | 0.276 | 0.112 | 0.25 |
| miR-302a | 35 | 29 | 0.82 | 0.638 | 0.607 | 0.258 | 0.214 |
| miR-302a* | 33 | 31 | 0.95 | 0.903 | 0.714 | 0.216 | 0.418 |
| miR-302b | 1 | 3 | 2.66 | 0.553 | 0.557 | 0.184 | 0.479 |
| miR-302b* | 19 | 22 | 1.14 | 0.649 | 0.607 | 0.111 | 0.411 |
| miR-302c | 157 | 130 | 0.83 | 0.323 | 0.387 | 0.161 | 0.477 |
| miR-302c* | 48 | 47 | 0.99 | 0.927 | 0.719 | 0.203 | 0.479 |
| miR-302d | 47 | 10 | 0.20 | 0.006 | 0.037 | 0.071 | 0.018 |
| miR-320 | 46930 | 39035 | 0.83 | 0.051 | 0.103 | 0.033 | 0.044 |
| miR-323 | 441 | 224 | 0.51 | 0.047 | 0.1 | 0.079 | 0.036 |
| miR-324-3p | 1723 | 1953 | 1.13 | 0.584 | 0.577 | 0.078 | 0.274 |
| miR-324-5p | 3129 | 5191 | 1.66 | 0.052 | 0.103 | 0.007 | 0.069 |
| miR-325 | 30 | 23 | 0.75 | 0.964 | 0.729 | 0.212 | 0.355 |
| miR-326 | 1908 | 686 | 0.36 | 0.003 | 0.023 | 0.007 | 0 |
| miR-328 | 449 | 210 | 0.47 | 0.062 | 0.117 | 0.061 | 0.054 |
| miR-330 | 94 | 460 | 4.92 | 0.012 | 0.05 | 0.005 | 0.016 |
| miR-331 | 342 | 493 | 1.44 | 0.354 | 0.406 | 0.122 | 0.192 |
| miR-335 | 12 | 78 | 6.42 | 0.224 | 0.297 | 0.045 | 0.2 |
| miR-337 | 4025 | 1855 | 0.46 | 0.006 | 0.037 | 0.023 | 0.007 |
| miR-338 | 455 | 31 | 0.07 | 0.011 | 0.05 | 0.004 | 0.006 |
| miR-339 | 121 | 258 | 2.12 | 0.089 | 0.151 | 0.079 | 0.159 |
| miR-340 | 3156 | 1157 | 0.37 | 0.002 | 0.018 | 0.004 | 0 |
| miR-342 | 23166 | 21226 | 0.92 | 0.595 | 0.577 | 0.212 | 0.274 |
| miR-345 | 213 | 764 | 3.58 | 0.095 | 0.159 | 0.025 | 0.155 |
| miR-346 | 34 | 35 | 1.01 | 0.879 | 0.709 | 0.201 | 0.438 |
| miR-361 | 489 | 583 | 1.19 | 0.616 | 0.594 | 0.079 | 0.439 |
| miR-367 | 85 | 62 | 0.73 | 0.457 | 0.486 | 0.199 | 0.401 |
| miR-368 | 964 | 917 | 0.95 | 0.659 | 0.614 | 0.25 | 0.316 |
| miR-369 | 632 | 599 | 0.95 | 0.429 | 0.463 | 0.256 | 0.24 |
| miR-370 | 634 | 258 | 0.41 | 0.002 | 0.018 | 0.01 | 0 |
| miR-371 | 6 | 28 | 4.59 | 0.021 | 0.062 | 0.003 | 0.023 |
| miR-372 | 727 | 431 | 0.59 | 0.030 | 0.074 | 0.035 | 0.078 |
| miR-373 | 246 | 44 | 0.18 | 0.007 | 0.039 | 0.04 | 0.001 |
| miR-373* | 282 | 116 | 0.41 | 0.068 | 0.127 | 0.125 | 0.076 |
| miR-374 | 218 | 46 | 0.21 | 0.002 | 0.022 | 0.042 | 0 |
| miR-375 | 1200 | 460 | 0.38 | 0.098 | 0.161 | 0.063 | 0.133 |
| miR-376a | 17 | 15 | 0.85 | 0.564 | 0.563 | 0.166 | 0.277 |
| miR-377 | 602 | 52 | 0.09 | 0.007 | 0.038 | 0.076 | 0.016 |
| miR-378 | 141 | 583 | 4.14 | 0.145 | 0.22 | 0.172 | 0.274 |
| miR-379 | 6 | 12 | 1.86 | 0.773 | 0.67 | 0.203 | 0.421 |
| miR-380-3p | 6 | 12 | 1.96 | 0.331 | 0.391 | 0.061 | 0.189 |
| miR-380-5p | 32 | 40 | 1.24 | 0.693 | 0.631 | 0.28 | 0.457 |
| miR-381 | 81 | 174 | 2.13 | 0.004 | 0.026 | 0.001 | 0.003 |
| miR-382 | 28 | 112 | 4.03 | 0.208 | 0.286 | 0.113 | 0.156 |
| miR-383 | 7 | 44 | 6.26 | 0.219 | 0.294 | 0.044 | 0.155 |
| miR-384 | 15 | 20 | 1.33 | 0.281 | 0.341 | 0.199 | 0.439 |
| miR-422a | 150 | 121 | 0.81 | 0.964 | 0.729 | 0.125 | 0.371 |
| miR-422b | 2828 | 5543 | 1.96 | 0.023 | 0.064 | 0.005 | 0.066 |
| miR-423 | 15257 | 1855 | 0.12 | 0.014 | 0.054 | 0.017 | 0.025 |
| miR-424 | 54 | 35 | 0.64 | 0.524 | 0.533 | 0.124 | 0.392 |
| miR-425 | 70 | 181 | 2.60 | 0.025 | 0.067 | 0.01 | 0.033 |
| D. melanog. miR-1 | 7 | 11 | 1.60 | 0.867 | 0.706 | 0.194 | 0.417 |
| D. melanog. miR-2a | 74 | 15 | 0.20 | 0.042 | 0.093 | 0.063 | 0.033 |
| D. melanog. miR-3 | 4 | 2 | 0.50 | 0.267 | 0.337 | 0.111 | 0.274 |
| D. melanog. miR-4 | 9 | 7 | 0.77 | 0.638 | 0.607 | 0.236 | 0.392 |
| D. melanog. miR-5 | 13 | 2 | 0.17 | 0.219 | 0.294 | 0.126 | 0.206 |
| D. melanog. miR-6 | 1377 | 885 | 0.64 | 0.379 | 0.419 | 0.188 | 0.267 |
| D. melanog. bantam | 3 | 7 | 2.06 | 0.761 | 0.663 | 0.079 | 0.289 |

*miRNAs numbered identically but distinguished by an asterisk are derived from different arms of the same precursor RNA.
**Probability that a particular miRNA is not differentially expressed, based on rank sum comparison of all 310 possible tumor normal pairs.
Wilcoxon, F. “Individual Comparisons by Ranking Methods.” Biometrics 1, 80-83, 1945.

1. A method of detecting differentially expressed miRNA in a nasopharyngeal carcinoma biological sample, comprising:
a) obtaining a nasopharyngeal carcinoma biological sample;
b) isolating RNA from the biological sample;
c) producing cDNAs from an isolated miRNA population from the biological sample;
d) transcribing the cDNAs to produce miRNA targets;
e) hybridizing the miRNA targets to an array of complementary probes for miRNA;
f) detecting the miRNA targets hybridized to the probe array; and
g) identifying differentially expressed miRNAs compared to a control,
wherein the differentially expressed miRNAs include one or more of miR-29c, miR-29a, miR-29b, miR-34c, miR-34b, miR-212, miR-216, miR-217, miR-151, and miR-192 miRNAs.

2. The method of claim 1, wherein the biological sample is a microdissected biological sample, a whole tissue section, or a biopsy.

3. The method of claim 1, wherein the biological sample is a cell culture sample.

4. The method of claim 1, wherein the biological sample is essentially free of stromal contaminants.

5. The method of claim 1 further comprising identifying a miRNA target mRNA, wherein the target mRNA has a nucleotide sequence complementary to a nucleotide sequence of an identified differentially expressed miRNA.

6. The method of claim 5, wherein the identified differentially expressed miRNA modulates the target mRNA expression levels.

7. The method of claim 6, wherein the target mRNA has an expression level inversely proportional to the identified differentially expressed miRNA.

8. The method of claim 7, wherein the target mRNA encodes an extracellular matrix protein.

9. The method of claim 8, wherein the extracellular matrix protein comprises at least one of COL4A1, COL4A2, COL3A1, COL1A2, COL5A2, FBN1, SPARC, COL15A1, COL1A1, and LAMC1.

10. The method of claim 1, wherein the step of detecting the miRNA targets comprises hybridizing capture sequences to the miRNA targets, the capture sequences comprising aggregated fluorophores.

11. A method of selecting a treatment for a nasopharyngeal carcinoma patient, the method comprising the steps of:
measuring miR-29c, miR-34b, miR-34c, miR-151, miR-192, miR-212, miR-216, and miR-217 miRNA expression levels in a diseased tissue sample taken from the nasopharyngeal area of a patient;
detecting differential expression of the miR-29c, miR-34b, miR-34c, miR-151, miR-192, miR-212, miR-216, and miR-217 miRNA expression levels in the patient; and
selecting a treatment for the nasopharyngeal carcinoma patient based on the differential expression levels of miR-29c, miR-34b, miR-34c, miR-151, miR-192, miR-212, miR-216, and miR-217 miRNAs,
wherein the treatment comprises administration of a therapeutically effective amount of a combination of chemotherapy and a selection of one or more of miR-29c, miR-29a, miR-29b, miR-34c, miR-34b, miR-212, miR-216, miR-217, miR-151, and miR-192 miRNAs.

12. The method of claim 11, wherein miR-29c, miR-34b, miR-34c, miR-212, miR-216, and miR-217 miRNA expression levels in the patient sample are reduced by at least 1/5-fold.

13. The method of claim 12, wherein miR-151 and miR-192 expression levels in the patient sample are increased by at least 20-fold.

14. A method for selecting a treatment for a nasopharyngeal carcinoma patient, the method comprising the steps of:
a) measuring miR-29c miRNA expression levels in an experimental sample taken from a patient;
b) measuring extracellular matrix mRNA expression levels in the experimental sample; and
c) identifying a treatment based on decreased miR-29c miRNA expression levels and elevated extracellular matrix mRNA expression levels in the sample,
d) wherein the treatment is administration of a therapeutically effective amount of a combination of chemotherapy and one or more of miR-29c, miR-29a, miR-29b, miR-34c, miR-34b, miR-212, miR-216, and miR-217 miRNAs to the nasopharyngeal carcinoma patient.

15. The method of claim 14, wherein the experimental sample is a tumorigenic tissue selected from the group consisting of dysplasia, anaplasia, and a precancerous lesion.

16. The method of claim 15, wherein the experimental sample is a microdissected tissue sample, a whole tissue section, a frozen tissue sample, or a biopsy.

17. The method claim 14, wherein miR-29c miRNA levels are decreased greater than 5-fold.

18. The method claim 14, wherein the extracellular matrix mRNA levels are upregulated by at least 2-fold.

19. The method of claim 18, wherein the extracellular matrix mRNAs encode at least one of COL4A1, COL4A2, COL3A1, COL1A2, COL5A2, FBN1, SPARC, COL15A1, COL1A1, and LAMC1.

20. The method of claim 19, wherein the extracellular matrix mRNAs encode COL4A1, COL4A2, COL3A1, COL1A2, COL5A2, FBN1, SPARC, COL15A1, COL1A1, and LAMC1.
